SEQUENCE LISTING (9 sequences)

SEQ ID NO: 1 - DNA, 21 bp, Artificial Sequence
  Description of Artificial Sequence Synthetic oligonucleotide
caagaagccagcagaggaatt

SEQ ID NO: 2 - DNA, 21 bp, Artificial Sequence
  Description of Artificial Sequence Synthetic oligonucleotide
  Description of Combined DNA/RNA Molecule Synthetic oligonucleotide
uuccucugcuggcuucuugtt

SEQ ID NO: 3 - DNA, 73 bp, Artificial Sequence
  Description of Artificial Sequence Synthetic oligonucleotide
gagcgctcgggacttgccgatagtggtgacggcggcaacatgtctgtggctttcgcggccccgaggcagcgag

SEQ ID NO: 4 - DNA, 18 bp, Artificial Sequence
  Description of Artificial Sequence Synthetic oligonucleotide
atggactacaaagacgat

SEQ ID NO: 5 - DNA, 53 bp, Artificial Sequence
  Description of Artificial Sequence Synthetic oligonucleotide
atacgatgttccagattacgcttctgtggctttcgcggccccgaggcagcgag

SEQ ID NO: 6 - DNA, 34 bp, Artificial Sequence
  Description of Artificial Sequence Synthetic oligonucleotide
atgtctgtggctttcgcggccccgaggcagcgag

SEQ ID NO: 7 - DNA, 73 bp, Artificial Sequence; identical to SEQ ID NO: 3
  Description of Artificial Sequence Synthetic oligonucleotide

SEQ ID NO: 8 - DNA, 73 bp, Unknown; identical to SEQ ID NO: 3
  Description of Unknown SS18 oligonucleotide

SEQ ID NO: 9 - DNA, 59 bp, Artificial Sequence
  Description of Artificial Sequence Synthetic oligonucleotide
ctcgggacttgccgatagtggtgacggcggcaacatgtctgtggctttcgcggccccga

(19) United States
(12) Patent Application Publication
(10) Pub. No.: US 2019/0338290 A1
(43) Pub. Date: Nov. 7, 2019

(54) TREATMENT OF SARCOMA

(71) Applicant: MEMORIAL SLOAN KETTERING CANCER CENTER, New York, NY (US)

(72) Inventors: Scott W. LOWE, New York, NY (US); Ana BANITO, New York, NY (US)

(73) Assignee: MEMORIAL SLOAN KETTERING CANCER CENTER, New York, NY (US)

(21) Appl. No.: 16/477,269

(22) PCT Filed: Jan. 16, 2018
(86) PCT No.: PCT/US18/13857

§ 371 Date: Jul. 11, 2019

Related U.S. Application Data
(60) Provisional application No. 62/446,597, filed on Jan. 16, 2017.

(51) Int. Cl. C12N 15/113; A61P 35/00; C12Q 1/6886

(52) U.S. Cl. CPC C12N 15/1137; A61P 35/00; C12Q 2600/154; C12N 2310/14; C12N 2310/531; C12Q 1/6886

(57) ABSTRACT

The present invention encompasses the recognition that epigenetic dependencies in sarcoma can be therapeutic targets. The present invention encompasses a method of treating sarcoma comprising the step of administering a PRC1.1 inhibitory agent to a subject suffering from or susceptible to sarcoma. In some embodiments, the PRC1.1 inhibitory agent is a KDM2B inhibitory agent, a BCOR inhibitory agent, and/or a PCGF1 inhibitory agent. In some embodiments, the PRC1.1 inhibitory agent reduces interaction of the SS18-SSX fusion protein with a polycomb repressive complex. In some embodiments, administration of the PRC1.1 inhibitory agent results in differentiation of synovial sarcoma cells into a more mesenchymal like state, comprising increased expression of COL1A1, SERPINE1 (PAI-1), ACTA2 (a-SMA), CDKN1A and/or CDKN2B.

### CROSS REFERENCE TO RELATED APPLICATIONS

This application claims priority to U.S. Provisional Patent Application No. 62/446,597, filed Jan. 16, 2017, which is hereby incorporated by reference in its entirety.

### BACKGROUND

Cancers, such as sarcomas, are often characterized by chromosomal translocations. The presence of such translocations can result in aberrations in cellular signaling and protein-protein interactions.

### SUMMARY

The present invention encompasses the recognition that chromosomal translocations can create proliferation dependency on particular protein-protein interactions, proteins, and or epigenetic changes. In some embodiments the present invention encompasses the recognition that sarcomas can be dependent on components of the PRC1.1 complex. In some embodiments the present invention encompasses the recognition that sarcomas can be dependent on KDM2B. In some embodiments the present invention encompasses the recognition that the KDM2B-PRC1 complex is a therapeutic target for the treatment of sarcomas.

In some embodiments, the present invention encompasses, among other things, a method of treating sarcoma comprising the step of administering a PRC1.1 inhibitory agent to a subject suffering from or susceptible to sarcoma. In some embodiments, the PRC1.1 inhibitory agent is a KDM2B inhibitory agent, a BCOR inhibitory agent, and/or a PCGF1 inhibitory agent.

In some embodiments, the PRC1.1 inhibitory agent reduces interaction of the SS18-SSX fusion protein with a polycomb repressive complex. In some embodiments, the PRC1.1 inhibitory agent reduces transcriptional activity induced by the SS18-SSX fusion protein. In some embodiments, the PRC1.1 inhibitory agent reduces interaction of the SS18-SSX fusion protein with CpG islands.

In some embodiments, the sarcoma is characterized by an SS18-SSX fusion protein. In some embodiments, the sarcoma is synovial sarcoma.

In some embodiments, the PRC1.1 inhibitory agent is a polypeptide, small molecule, or nucleic acid. In some embodiments, the PRC1.1 inhibitory agent is an shRNA. In some embodiments, the PRC1.1 inhibitory agent is an antibody agent.

In some embodiments, the PRC1.1 inhibitory agent results in reduced proliferation of cancer cells. In some embodiments, administration of the PRC1.1 inhibitory agent results in cell cycle arrest. In some embodiments, administration of the PRC1.1 inhibitory agent results in differentiation of synovial sarcoma cells into a more mesenchymal like state. In some embodiments, increased expression of COL1A1, SERPINE1 (PAI-1), ACTA2 (α-SMA), CDKN1A and/or CDKN2B indicate a more mesenchymal like state.

In some embodiments, the present invention encompasses, among other things, a method of treating sarcoma comprising the step of administering a KDM2B inhibitory agent to a subject suffering from or susceptible to sarcoma. In some embodiments, the step of administering a KDM2B inhibitory agent comprises administering a KDM2B inhibitory agent to a subject in whom a KDM2B dependency has been detected. In some embodiments, the step of administering comprises administering a KDM2B inhibitory agent to a subject in whom a KDM2B-SS18-SSX dependency has been detected. In some embodiments, a KDM2B inhibitory agent reduces the level or activity of KDM2B. In some embodiments, a KDM2B inhibitory agent targets the ZF-CXXC domain of KDM2B. In some embodiments, a KDM2B inhibitory agent reduces the interaction of KDM2B and SS18-SSX. In some embodiments, a KDM2B inhibitory targets a RAWUL domain of PCGF1. In some embodiments, a KDM2B inhibitory agent reduces the interaction of KDM2B and PRC1. In some embodiments, a cancer treated by the methods and compositions of the present invention is characterized by KDM2B dependency. In some embodiments, a cancer treated by the methods and compositions of the present invention is characterized by an SS18-SSX fusion protein. In some embodiments, a cancer treated by the methods and compositions of the present invention is characterized by decreased methylation at Histone 3 lysine 27 trimethylation (H3K27me3) relative to a reference. In some embodiments, a reference is healthy tissue from the subject. In some embodiments, a cancer treated by the methods and compositions of the present invention is a sarcoma. In some embodiments, a cancer treated by the methods and compositions of the present invention is synovial sarcoma. In some embodiments, a KDM2B inhibitory agent of the present invention is a polypeptide, small molecule, or nucleic acid. In some embodiments, a KDM2B inhibitory agent of the present invention is an shRNA. In some embodiments, a KDM2B inhibitory agent of the present invention is an antibody agent. In some embodiments, administration of a KDM2B inhibitory agent results in reduced proliferation of cancer cells.

In some embodiments, the present invention encompasses a method of detecting KDM2B dependency in a subject. In some embodiments, the present invention encompasses a method of detecting KDM2B dependency in a cancer in a subject by detection of H3K27me3. In some embodiments, H3K27me3 is decreased in the subject relative to a reference. In some embodiments, the present invention encompasses a method of detecting KDM2B dependency in a cancer in a subject by detection of interaction of KDM2B and SS18-SSX.

In some embodiments, the present invention encompasses a method of identifying and or characterizing a PRC1.1 inhibitory agent. In some embodiments, PRC1.1 inhibitory agent is identified as disrupting the association of the SS18-SSX fusion protein and a component of a PRC1.1 complex.

### BRIEF DESCRIPTION OF THE DRAWING

FIG. 1, comprising panels A through F, demonstrates an shRNA screen for epigenetic dependencies in synovial sarcoma. (A) shRNA screen strategy. A library against 400 genes encoding chromatin remodelers was screened in two different cells lines: a mouse synovial sarcoma cell line (M5SS1) derived from a mouse model of synovial sarcoma where the human SS18-SSX2 oncogene is conditionally expressed in the myogenic linage and in mouse myoblasts (C2C12). shRNA representation was evaluated by next generation sequencing three days after transduction of the shRNA library (t0) and following serial passages at day 16 (tfinal) following transduction. (B) Differences in shRNA representation are presented as log2 of the ration between reads at Tfinal (D16) compared with T0 (D3). shRNAs against Rpa3 and Myc were used as positive controls as they are required for proliferation in both cell lines under study. shRNA against Renilla and luciferase (shRen and shLuc) were used as neutral control hairpins. Three shRNAs against SS18-SSX are on the top of the depletion hits in the synovial sarcoma cell line, but are neutral in myoblast demonstrating specific addiction for the fusion oncogene for proliferation of sarcoma cells. Three shRNAs targeting KDM2B specifically deplete in synovial sarcoma but have no effect in normal myoblasts, suggesting a specific dependency on this epigenetic remodeler. (C) Validation of hits in M5SS1 sarcoma cell lines comparing the results obtained in the screen (blue bars) and in a one by one validation experiment (red bars) shRNA expression is linked to GFP allowing to track percentage of GFP positive cells overtime. Relative % of GFP positive cells relative to t0 is plotted. shRNAs against SS18, KDM2B and Bcor (another member of the PRC1.1 complex) are highlighted. (D) Validation of KDM2B shRNAs in a different expression plasmid with improved knockdown showing kinetics of KDM2B depletion in mouse synovial sarcoma cells. (E) Crystal violet stained plates showing differences in cell proliferation in M5SS1 and C2C12. Ten thousand cells were plated at T0 and plates were fixed and stained 15 days later. Knockdown of SS18-SSX or KDM2B results in inhibition of cell proliferation in M5SS1, having no effect in normal myoblast (C2C12). (F) Western blot analysis of M5SS1 protein lysates showing efficient knockdown of KDM2B. ** pvalue<0.001, ***pvalue<0.0001.

FIG. 2, comprising panels A through I, demonstrates KDM2B is required for proliferation of human synovial sarcoma in vitro and in vivo. (A) Three shRNAs against human KDM2B were designed and validated. Effect of knockdown of KDM2B in a patient derived synovial sarcoma cell line positive for the SS18-SSX translocation (HS-SY-II). Relative percentage of percentage of GFP to T0 is plotted. An shRNA against the first translocated gene of the oncogenic fusion (SS18.273) and against the second gene (SSX. 1274) was used to show proliferation of these cells depends of the oncogenic fusion. (B) Western blot analysis of HS-SY-II protein lysates showing efficient knockdown of KDM2B. (C) Crystal violet stained plates showing differences in cell proliferation in HS-SY-II upon SS18-SSX or KDM2B knockdown. Twenty thousand cells were plated at t0, fixed and stained 15 days later. (D) Bright field images showing morphological differences in HS-SY-II cell line upon SS18-SSX or KDM2B knockdown in vitro. (E) GFP staining of tumors generated by subcutaneous injection of HS-SY-II cell lines transduced with the indicated shRNAs. As shown, tumors generated with shRen control retained GFP expression (liked to shRNA expression) while tumors generated with shSSX or shKDM2B, are mostly GFP negative. (F) Tumor volume was measure over time in HS-SY-II xenografts. Not only were the tumors GFP negative but knockdown of KDM2B or SSX significantly inhibited tumor growth. (G) Tumor weight at final time point in xenografts generated with HS-SY-II (left graph) and SYO-I (right graph) synovial sarcoma cell lines. (H) Quantification of KDM2B IHC in sarcoma tissue microarrays showing higher KDM2B levels in synovial sarcoma tumors (n=57) when compared with benign and other sarcoma sub-types. (I) KDM2B staining of a positive synovial sarcoma sample and a negative sample showing specific nuclear staining. Malignant peripheral nerve sheath tumor (MPNST).

FIG. 3, comprising panels A through F, demonstrates, the ZF-CXXC domain of KDM2B and PRC1.1 are required for synovial sarcoma proliferation. (A) KDM2B protein domains in long and short isoforms. Guide RNAs where design against the first two exons of the gene, to target the JmJC domain required for histone demethylase activity and the ZF-CXXC domain required for DNA binding (to recruit PRC1.1 to CpG islands. (B) Depletion assays in five different human synovial sarcoma lines. Guide RNAs against the first exons and the JmJC domain showed little effect over cell proliferation while guides against the CXXC consistently affected proliferation. These results suggest that the ZF-CXXC and the short KDM2B isoform are required for proliferation of synovial sarcoma cells, while the demethylase activity (JmJC domain) is dispensable. (C) T7 assay showing efficient editing using the guide RNAs against the different KDM2B domains. (D) Guide RNAs were designed against the first exon of PCGF1 (a specific member of the PRC1.1 complex), and against the RAWUL domain. The latter is important for interaction with BCOR and BCORL1, and consequent KDM2B assembly into the PRC1.1 complex. (E) T7 assays showing efficient editing by guide RNAs against PCGF1. (F) Specifically targeting the RAWUL domain inhibits proliferation of HS-SY-II cells, suggesting that targeting this protein domain and interaction between PCGF1 and BCOR/BCORL1 could be an effective therapeutic strategy for synovial sarcoma treatment.

FIG. 4, comprising panels A through C, demonstrates SS18-SSX and KDM2B knockdown lead to similar expression changes and abolish a synovial sarcoma gene signature. (A) Gene set enrichment analysis (GSEA) of genes up-regulated and down-regulated by shKDM2B showing that the gene expression changes induced by KDM2B correlated with the ones induced by shSS18-SSX. The same analysis was done for genes upregulated or downregulated by SS18-SSX (graphs on the right side). (B) Unsupervised clustering of gene expression mean RNA-seq values from TCGA data for the sarcoma set. Ten synovial sarcomas (SYNSARC), 72 leiomyosarcomas (LMS), 32 uterine leiomyosarcomas (Uterine LMS), 10 malignant peripheral nerve sheath tumors (MPNST), 58 dedifferentiated liposarcomas (DEDIFF-LPS), 30 Pleomorphic Malignant Fibrous Histiocytomas (MFH/PLEIO), 25 Myxofibrosarcomas (MFS), and 22 undifferentiated pleiomorphic sarcomas (UPS) are included. (C) Supervised clustering of the 200 genes differentially up-regulated and down-regulated upon KDM2B knockdown showing inhibition of genes belonging to the synovial sarcoma (SS) signature upon both shSS18-SSX and shKDM2B.

FIG. 5, comprising panels A through K, demonstrates SS18-SSX and KDM2B co-occupy the same genomic regions encoding developmental transcription factors (A) Endogenous SS18-SSX1 of HS-SY-II was tagged with a Flag-HA using CRISPR/Cas9 editing HDR. A guide RNA targeting the region around the ATG and a DNAss containing homology arms for the N-terminal region of SS18 and a flag-HA sequence was used as template. For screening positive colonies PRC primers flanking the ATG site of SS18 were used. A clone with HA-Flag in the SS18-SSX allele but with wild-type SS18 unaffected was used for further analysis. (B) Crystal violet stained plates showing that the HS-SY-II HA-SS18-SSX1 tagged clone is still sensitive to either knockdown of the SS18-SSX1 or KDM2B. (C) Immunofluorescence analysis showing nuclear staining of HA tag. Knockdown of SS18-SSX1 using an shRNA against the second partner of the fusion results in loss of nuclear staining, demonstrating specific tagging of the SS18-SSX1 translocation. (D) Western blot analysis for HA tag showing knockdown of the HA-SS18-SSX with either shRNAs against SS18 or SSX1. (E) Heat maps showing HA-SS18-SSX1 and KDM2B ChIP-Seq signals over the 10,932 HA-enriched regions identified in HA-SS18-SSX HS-SY-II cells. Rows correspond to ±5-Kb regions across the midpoint of each HA-enriched region, ranked by increased HA-SS18-SSX signal in the tagged clone. Color shading corresponds to the HA-SS18-SSX and KDM2B ChIP-Seq read count in each region. As depicted HA-SS18-SSX and KDM2B co-occupy the same genomic regions. (F) scatterplots of absolute HA-SS18-SSX1 and KDM2B signals (tag counts) at 10,533 SS18-SSX/KDM2B co-occupied regions, showing quantitative correlation of binding between the two proteins. Some of the genes with highest occupancy are highlighted. (G) Chip-seq validation of SS18-SSX binding at the EN2, LHX3 and UNCX loci. Two sets of primers were designed to amplify the CGIs at the promoters of the those genes. Primers for the promoter of B-Globulin and an intron region of BCL6 were used as negative controls. (H) Gene tracks for MNX1 and LHX3 showing co-occupancy of SS18-SSX and KDM2B. Gene tracks for a negative control (HS-SY-II untagged parental cell line) are also shown. (I, J) Gene set enrichment analysis GSEA comparing the expression of genes associated with the top 500 regions occupied by SS18-SSX in synovial sarcoma when compared to other sarcoma types (I) and upon knockdown of SS18-SSX (J). (K) Gene ontology analysis of genes associated with the top 500 regions occupied by SS18-SSX1, showing they are highly enriched in developmental proteins, mostly transcription factors. A large number correspond to homeobox genes (eg. EN2, LHX3, UNCX, MNX1) involved to neurogenesis.

FIG. 6, comprising panels A through H, demonstrates SS18-SSX interacts with KDM2B and PRC1.1. (A) Immunofluorescence images showing proximity ligation assay (PLA) results for SS18-SSX and KDM2B in FUJI and Yamato-SS synovial sarcoma cell lines. TLE1 was used as a positive control. MFC7 cells, which are negative for the SS18-SSX translocation, were used as a negative control. (B) Quantification of TLE1 and KDM2B PLA (showing number of foci per nucleus) in the different cell lines analyzed. (C) Co-immunoprecipitation using KDM2B antibody showing specific interaction with SS18-SSX in HS-SY-II cell line. (D) PLA results are specific since knockdown of SS18-SSX leads to loss of PLA positive nuclear foci. (E) Western blot analysis showing efficient knockdown of SS18-SSX using siRNA against SSX1. (F) Quantification of PLA results for SS18-SSX knockdown in HSSY-II. (G) Co-IP for HA tag using the HA-SS18-SSX tagged clone described in FIG. 5A-D, showing interaction of HA-SS18-SSX with KDM2B and members of the PRC1.1 complex (BCOR and PCGF1). (H) PLA results using an HA tag antibody verifying KDM2B interaction in the HA tagged clone.

FIG. 7, comprising panels A through F, demonstrates KDM2B is required for SS18-SSX occupancy on chromatin. (A) Scatterplot showing correlation between differential SS18-SSX occupancy upon knockdown of SS18-SSX1 and KDM2B on 10,533 SS18-SSX/KDM2B co-occupied regions. (B) HASS18-SSX ChIP-Seq enrichment meta-profiles in shREN, shSS18-SSX and shKDM2B conditions representing the average read counts per 20-bp bin across a 10-Kb window centered on 4,567 shSS18-SSX sensitive regions. (C) Box plots of fold change difference upon shKDM2B in 10,533 SS18-SSX/KDM2B co-occupied regions and 451 KDM2B non-occupied regions, showing KDM2B knockdown primarily affects SS18-SSX occupancy at KDM2B bound regions. (D) Gene track depicting KDM2B (red), HA-SS18-SSX (blue) and H3K27me3 peaks at the UNCX locus. (E) Scatterplot showing correlation between differential H3K27me3 repressive mark upon knockdown of SS18-SSX1 and KDM2B on 10,533 SS18-SSX/KDM2B co-occupied regions. Genes with highest gains in H3K27me3 are highlighted. (F) Proposed model for KDM2B requirement in synovial sarcoma. In synovial sarcoma KDM2B binds unmethylated CpG islands of developmental genes. By interacting with SS18-SSX it allows its recruitment and subsequent aberrant activation of developmental transcription factors. Upon KDM2B inhibition SS18-SSX binding is decreased, allowing H3K27me3 gains at a sub-set of SS18-SSX targets and consequent down-regulation of expression of developmental TFs, presumably allowing re-establishment of normal differentiation programs.

FIG. 8, comprising panels A through D, demonstrates that KDM2B is an epigenetic dependency in synovial sarcoma. (A, B) Differences in shRNA representation presented as logₑ of the ratio between average reads at Tf (Day 16) and T₀ (Day 3) in synovial sarcoma cells (M5SS1) (A) and myoblasts (C2C12) (B). Plotted values correspond to the average of three independent replicates. shRNAs against Renilla and luciferase (Ren. 713 and Luc. 1309) were used as neutral control hairpins. (C) Clonogenic assay of M5SS1 (upper panel) and C2C12 (lower panel) cells transduced with the indicated shRNAs. (D) Validation of the effect of KDM2B shRNAs showing kinetics of KDM2B depletion in mouse synovial sarcoma cells. Data presented as mean±s.d. (n=2) ** pvalue<0.001, ***pvalue<0.0001, unpaired t-test.

FIG. 9, comprising panels A through I, demonstrates that KDM2B inhibition irreversibly triggers mesenchymal differentiation. (A) Representative image of a KDM2B positive synovial sarcoma sample showing specific immunohistochemical (IHC) nuclear staining and of a negative sample (clear cell sarcoma) within the same set of sarcoma tissue microarrays (TMAs) analyzed. Scale bar=100 μm. (B) Quantification of KDM2B IHC in sarcoma TMAs showing H-scores for KDM2B staining in synovial sarcoma tumors (n=58), malignant peripheral nerve sheath tumors (MPNST) (n=76), other translocation driven sarcomas (Translocation) (n=108), other sarcoma sub-types in the differential diagnosis for synovial sarcoma (Other) (n=209) and benign soft tissue tumors (Benign) (n=89). Box whiskers represent mean H-scores ±10 and 90 percentiles. (C) Immunoblot for KDM2B in HS-SY-II cells transduced with the indicated shRNAs. (D) Cell competition assay for GFP-linked shRNAs against KDM2B and SS18-SSX in the HS-SY-II human synovial sarcoma cell line. Relative percentage of GFP+ cells relative to T0 (3 days following shRNA activation) is plotted; data is presented as mean±s.d. (n=2). (E) Clonogenic assay of HS-SY-II, YaFUSS and SYO-1 cells transduced with the indicated shRNAs. (F) Quantitative RT-PCR for fibroblasts-related genes. IMR90 human diploid fibroblasts were used as a positive control. Data presented as mean±s.d. (n=2) (G) Immunofluorescence analysis of alpha smooth muscle actin (α-SMA) in HS-SY-II cells, ten days following transduction with the indicated shRNAs. Scale bar=20 μm. (H) Bright field and GFP images of HS-SY-II cells transduced with TRE-regulated shRNAs growing in the presence of doxycycline (Dox+) or upon doxycycline withdrawal (Dox−), at day 10 after Dox withdrawal. Scale bar=25 μm (I) Growth curves for HS-SY-II cells continuously growing in doxycycline media or after doxycycline withdrawal. Data presented as mean±s.d. (n=2). *pvalue<0.05, **pvalue<0.005 ***pvalue<0.0005. B. Kruskal-Wallis 1-way ANOVA. D, F, I unpaired t-test.

FIG. 10, comprising panels A through E, demonstrates KDM2B is required for synovial sarcoma maintenance in vivo. (A) Strategy to evaluate the effect of KDM2B knockdown in vivo in HS-SY-II and SYO-1-derived xenografts. See also STAR methods. (B) Tumor volume was measured over time in HS-SY-II xenografts transduced with the indicated shRNAs. Error bars correspond to mean±s.e.m (n=10). (C) Tumor weight at the final time point for xenografts generated with HS-SY-II (left) and SYO-1 (right) cell lines. Data represented as mean±s.d. (n=10). (D) Bright field and GFP images of tumors generated by subcutaneous injection of HS-SY-II cells transduced with the indicated inducible shRNAs. (E) GFP IHC staining of tumors generated by subcutaneous injection of HS-SY-II cells transduced with the indicated shRNAs. Scale bar=150 μm B, C, and D. Two-tailed t-test *pvalue<0.05, **pvalue<0.005, ***pvalue<0.0005.

FIG. 11, comprising panels A through F, further confirms the DNA binding domain of KDM2B and the non-canonical PRC1.1 complex are important for synovial sarcoma proliferation. (A) Schematics showing human KDM2B JmjC (demethylase activity) and ZF-CxxC (binds unmethylated CpG islands) protein domains in long and short KDM2B isoforms and location of single guide RNAs (sgRNA) used. (B) Depletion assays based on the % of GFP+ cells at day 3 and at 21 days following sgRNA infection in HS-SY-II and YaFUSS human synovial sarcoma lines. Error bars correspond s.d of two independent experiments. (C) Bright field images of HS-SY-II cells transduced with the indicated shRNAs 10 days following shRNA activation. Scale bar=25 μm (D) Clonogenic assay of HS-SY-II cells transduced with the indicated shRNAs. (E) Quantitative RT-PCR showing efficient gene knockdown of KDM2B, PCGF1 and BCOR. Error bars correspond s.d (n=2). (F) Schematics of guide RNAs designed against the first exon of PCGF1, and against the RAWUL domain of PCGF1 (surrounding Valine 206) (upper panel). Depletion assays based on the % of GFP+cells at day 3 and at 18 days following sgRNA infection in HS-SY-II cells. Error bars correspond s.d (n=2)(lower panel). B, E, F. Unpaired t-test *pvalue<0.05, **pvalue <0.005, ***pvalue<0.0005.

FIG. 12, comprising panels A through H, demonstrates that endogenous SS18-SSX interacts with PRC1.1. (A) Endogenous SS18-SSX1 of the HS-SY-II human synovial sarcoma cell line was tagged with Flag-HA epitopes using CRISPR/Cas9 editing-mediated homology-directed repair (HDR). An sgRNA targeting the region around the ATG and an ssDNA containing homology arms for the N-terminal region of SS18 and a Flag-HA sequence was used as template. For screening positive colonies PCR primers flanking the ATG site of SS18 were used (represented as arrows). A clone with Flag-HA in the SS18-SSX allele but with the wild-type SS18 allele unaffected was used for further analysis. (B) Immunofluorescence analysis showing nuclear staining using an anti HA tag antibody. Knockdown of SS18-SSX1 using an shRNA against the SSX component of the fusion results in loss of nuclear staining. Scale bar=25 μm (C) Western blot analysis for HA tag in HA-SS18-SSX cells with the indicated shRNAs. (D) Co-IP analysis using an anti-HA tag antibody in HA-SS18-SSX tagged clone described in FIG. 12A-C. HS-SY-II parental untagged line was used as a negative control. (E) Co-IP using an anti-KDM2B antibody in HA-SS18-SSX tagged clone described in FIG. 12A-C, showing interaction of KDM2B with members of the PRC1.1 complex (BCOR and PCGF1) in HS-SY-II and 293T cells, and with SS18-SSX1 in the HA-SS18-SSX tagged clone. (F) Proximity ligation assay images and respective quantification verifying KDM2B and SS18-SSX in situ co-localization using an SS18 specific antibody or an HA tag antibody in untagged (parental) and HA tagged HS-SY-II clone. Scale bar=25 μm (G) Co-IP using an anti-KDM2B antibody in 293T cells expressing HA-tagged versions of wild type (WT) SS18 and SSX1; and controls (HA-GFP and HA-SS18-SSX). (H) Co-IP using an anti-KDM2B antibody in 293T cells expressing GFP fused to the last 78 aminoacids of SS18-SSX1 (SSX1 fragment), and the same fragment lacking the SSXRD domain.

FIG. 13, comprising panels A through I demonstrates that SS18-SSX and KDM2B co-occupy and regulate genes that define a synovial sarcoma signature. (A) Heat maps showing HA-SS18-SSX1, BRG1 and KDM2B ChIP-Seq signals over the 10,984 HA-enriched regions identified in HA-SS18-SSX tagged cells. Rows correspond to ±10-Kb regions across the midpoint of each HA-enriched region, ranked by increasing HA-SS18-SSX signal in the tagged clone. Color shading corresponds to the HA-SS18-SSX, BRG1 and KDM2B ChIP-Seq read counts in each region. (B) Scatterplot of absolute HA-SS18-SSX and KDM2B signals (tag counts) at 10,533 SS18-SSX/KDM2B co-occupied regions, showing quantitative correlation of SS18-SSX and KDM2B binding. (C) KDM2B (left) and SS18-SSX (right) binding profiles centered on TSS for CpG-rich promoters and CpG-poor promoters. The average signal for all annotated genes of the two groups within 5 kb genomic regions flanking TSSs is shown. (D) Gene tracks for LHX3 showing co-occupancy of SS18-SSX, BRG1 and KDM2B at CGI rich regions. HA-ChIP for a negative control (HS-SY-II untagged parental cell line) is also shown. (E) Average methylation (beta) values for regions inside (y-axis) and outside (x-axis) SS18-SSX/KDM2B occupied regions. Each data point corresponds to an individual patient sarcoma sample. Different sarcoma sub-types are indicated and color-coded. Synovial sarcomas (SS), undifferentiated pleiomorphic sarcomas (UPS), Myxofibrosarcomas (MFS), malignant peripheral nerve sheath tumors (MPNST), uterine leiomyosarcomas (Uterine LMS), leiomyosarcomas (LMS), dedifferentiated liposarcomas (DDLPS). (F) Methylation (beta) values across the LHX3 locus in SS compared with normal tissue (Fat) and all other sarcoma subtypes described in FIG. 13E. (G) Gene ontology analysis for Biological Process category of genes associated with all 10,533 SS18-SSX/KDM2B occupied regions. (H) Unsupervised clustering based on mean RNA-Seq values of sarcoma sub-types analyzed by The Cancer Genome Atlas (TCGA) (sarcoma set). Synovial Sarcoma (SYNSARC, n=10), LMS (n=72), Uterine LMS (n=32), MPNST (n=10), DEDIFF-LPS (n=58), MFH/PLEIO (n=30), MFS (n=25), and 22 UPS (n=22). (I) Supervised clustering of the 200 genes differentially upregulated or downregulated upon KDM2B knockdown. C. Unpaired t-test, ***pvalue<0.0001.

FIG. 14 comprising panels A through F, demonstrates KDM2B recruits SS18-SSX to activate developmentally regulated genes otherwise subjected to polycomb-mediated gene repression. (A) HA-SS18-SSX and BRG1 (B) ChIP-Seq enrichment meta-profiles in Ren.713 (control shRNA), SSX.1274 and KDM2B. 4395 conditions representing the average read counts per 20-bp bin across a 20-Kb window centered on 4,567 SSX. 1274 sensitive regions. (C) Gene track depicting KDM2B (red), HA-SS18-SSX (blue) and H3K27me3 (black) ChIP-Seq and ATAC-Seq (purple) peaks at the MNX1 and S100A2/4 loci. (D) Scatterplot showing correlation between differential H3K27me3 levels upon knockdown of SS18-SSX1 and KDM2B at SSX.1274 sensitive regions. Genes with highest gains in H3K27me3 are highlighted. (E, F) ATAC-Seq enrichment meta-profiles in Ren.713 (control shRNA), SSX.1274 and KDM2B. 4395 conditions representing the average read counts per 20-bp bin across a 10-Kb window centered on 10,984 SS18-SSX occupied regions (E) and at 117,459 non-SS18-SSX occupied ATAC-Seq peaks (F). Unpaired t-test ***pvalue<0.0001.

FIG. 15, comprising panels A through E, describes an shRNA screen to find epigenetic dependencies in synovial sarcoma. (A) shRNA screen strategy. A library against 400 genes encoding chromatin remodelers was screened in triplicate in two different cell lines: mouse synovial sarcoma (M5SS1) and mouse myoblasts (C2C12). shRNA representation was evaluated by next generation sequencing three days after transduction of the shRNA library (T₀) and following serial passages at day 16 (T final) after transduction. (B) Validation of hits in C2C12 (upper graph) and synovial sarcoma (lower graph) cell lines. Changes in shRNA representation (Screen) and relative % of GFP positive, shRNA-expressing cells, relative to T₀ (Validation) were plotted. shRNAs against SS18 and Kdm2b are highlighted. The dashed line corresponds to 2-fold depletion. (C) Bright field images of M5SS1 cells showing changes in morphology upon SS18-SSX knockdown (SS18 shRNAs are against the human gene and therefore do not target the wild-type mouse SS18) and Kdm2b knockdown. GFP-low colonies that escape shRNA expression do not exhibit the same morphological changes. Scale bar=50 μm. (D) Western blot analysis of KDM2B in M5SS1 protein lysates (Kdm2b shRNAs 433, 2848 and 3145). (E) Rescue experiments showing comparison of knockdown of SS18-SSX or of Kdm2b in M5SS1 cells transduced with an empty vector control (MSCV) or a Kdm2b cDNA. Percentage of GFP+ cells at day 16 relative to T0 is plotted.

FIG. 16, comprising panels A through F, further demonstrates human synovial sarcoma cells depend on KDM2B (A) Quantitative RT-PCR in IMR90 human fibroblasts and human macrophages (hMac) (black), human synovial sarcoma cells positive for SS18-SSX2 (red), or SS18-SSX1 (blue) gene fusions and other cancer cells lines (gray) not detected (nd). (B) c-MYC, KRAS and KDM2B CRISPR/Cas9 screen data from project Achilles in 33 solid cancer cell lines (Pancreas, lung, Colon, ovary and bone). Color shading corresponds to sgRNAs scoring levels as described in https://portals.broadinstitute.org/achilles with an increased green intensity indicating greater depletion. (C) Bright filed images of HS-SY-II transduced with the indicated shRNAs 8 days following shRNA activation. (D) Schematics for evaluating reversibility of effects induced by SS18-SSX or KDM2B depletion (Top panel). HS-SY-II cells were transduced with the indicated TRE-driven shRNAs linked to GFP. Following 10 days of shRNA expression, GFP positive cells were sorted and re-plated. On Day 12 cells were seeded and maintained in the presence or absence of doxycycline for further analysis. (E) Quantitative q-PCR for HS-SY-II cells grown in the presence or absence of doxycycline, at day 10 after Dox withdrawal. (F) GFP IHC staining of tumors generated by subcutaneous injection of SYO-1 cell line transduced with the indicated shRNAs. A., E. Unpaired t-test *pvalue<0.05, **pvalue<0.005***, pvalue<0.0005.

FIG. 17, comprising panels A through J,. demonstrates the DNA binding domain of KDM2B is critical for synovial sarcoma maintenance. (A) T7 assay showing efficient gene editing using the guide RNAs against the different KDM2B genomic regions. (B) Depletion assays based on the number of GFP+ cells at day 3 and at 21-24 days following sgRNA infection in Yamato-SS, SYO-1 and FUJI human synovial sarcoma lines. Error bars correspond to means s.d. (n=2). (C) Clonogenic assay of HS-SY-II cells transduced with the indicated shRNAs and MSCV-neo empty vector control, wild-type mouse Kdm2b (Kdm2b^{WT}), a JmjC-deficient mutant (Kdm^{H211A/H222A}) and a ZF-CxxC-deficient mutant (Kdm2b^{C600A/C603A}). (D) Crystal violet quantification of the clonogenic assays presented in (C). Data is represented as mean±s.d. (n=2). (E) Immunoblot analysis of total KDM2B levels and exogenous KDM2B (Myc-tag) levels (* indicates an unspecific band). (F) Clonogenic assay of HS-SY-II cells transduced with the indicated shRNAs and MSCV-hygro empty vector control and the short isoform of Kdm2b (Kdm2b short IF). (G) Immunoblot analysis of total KDM2B levels in cells over expressing KDM2B's short isoform. (H) Clonogenic assay of IMR90 normal human diploid fibroblasts transduced with the indicated shRNAs (I) T7 assays showing comparable and efficient editing by all guide RNAs against PCGF1. (J) Bright field images of HS-SY-II cells transduced with a control sgRNA (GFP) and an sgRNA against the RAWUL domain of PCGF1. sgRNA positive cells were selected after puromycin treatment and images were acquired 8 days later. Scale bar=25 μm. D. Unpaired t-test *pvalue<0.05, **pvalue<0.005.

FIG. 18, comprising panels A through D, demonstrates that SS18-SSX interacts with KDM2B via the SSX repressor. (A) Clonogenic assay of the HA-SS18-SSX tagged HS-SY-II clone described in FIG. 12A-12C, transduced with the indicated shRNAs. (B, C). Proximity ligation assay images and respective quantification verifying KDM2B and SS18-SSX in situ co-localization using (B) an SS18 specific antibody in MFC7 cells and indicated synovial sarcoma lines; and (C) using an SS18 antibody in HS-SY-II cells upon SS18-SSX knockdown. Data correspond to means±s.d. after quantification of three independent fields. Scale bar=25 μm. (D) Co-IP using an anti-KDM2B antibody in 293T cells transiently expressing HA-tagged versions of C-terminal SS18-SSX deletions mutants. Unpaired t-test, **pvalue<0.005, ***pvalue<0.0005.

FIG. 19, comprising panels A through H, demonstrates SS18-SSX/KDM2B bind and activate synovial sarcoma-signature genes. (A) Heat maps showing KDM2B, HA-SS18-SSX1 and BRG1 ChIP-Seq signals over 11,345 KDM2B-enriched regions. Rows correspond to ±5-Kb regions across the midpoint of each KDM2B-enriched peak, ranked by increasing KDM2B ChIP signal. Color shading corresponds to KDM2B, HA-SS18-SSX, and BRG1 ChIP-Seq read counts in each region. (B) ChIP-qPCR validation of SS18-SSX binding at the EN2, LHX3 and UNCX loci using two sets of primers design to amplify the regions corresponding to CGIs. Primers for the promoter of B-Globulin and an intronic region of BCL6 were used as negative controls. (C) Methylation (beta) values across the MNX1 locus in SS compared with normal tissue (Fat) and all other sarcoma subtypes described in FIG. 6E. (D) Methylation (beta) values in all 10,533 5518-SSX/KDM2B co-occupied regions, comparing the top 500 regions with highest (red), top 500 with the lowest (green) and medium (blue) SS18-SSX/KDM2B binding. *** pvalue<0.001. (E, F) Gene set enrichment analysis (GSEA) comparing the expression of genes associated with the top 500 regions occupied by SS18-SSX in synovial sarcoma when compared to other sarcoma types (E) and upon knockdown of SS18-SSX or of KDM2B (F). (G) Gene set enrichment analysis comparing expression of genes differentially expressed in HS-SY-II cells transduced with KDM2B. 4395 and SSX. 1274. (H) Gene ontology analysis of genes commonly down regulated by SS18-SSX or KDM2B knockdown. (G) Plotted RNA-Seq fold changes of genes downregulated by KDM2B or SS18-SSX knockdown. (H) Quantitative RT-PCR validating gene expression results obtained by RNA-Seq for downregulated genes.

FIG. 20, comprising panels A through F, demonstrates gene repression is a less prominent feature mediated by SS18-SSX. (A) Plotted RNA-Seq fold changes of genes downregulated by KDM2B or SS18-SSX knockdown. (B) Quantitative RT-PCR validating gene expression results obtained by RNA-Seq for downregulated genes. (C) Percentage of genes identified as SS18-SSX targets by ChIP in all upregulated genes (log₂ FC≥1) and all downregulated genes (log₂ FC≤−1) in response to SSX.1274 in HS-SY-II cells. (D) Gene set enrichment analysis comparing levels of SS18-SSX ChIP signal for downregulated (left) or upregulated (right) genes as a result of SS18-SSX knockdown. (E) Gene ontology analysis of genes commonly upregulated upon SS18-SSX or KDM2B knockdown. (F) Plotted RNA-Seq fold changes of genes upregulated by KDM2B or SS18-SSX knockdown.

FIG. 21, comprising panels A through F, demonstrates that SS18-SSX and KDM2B inhibition induce changes in gene accessibility and BRG1 chromatin occupancy. (A) Scatterplot showing correlation between differential SS18-SSX occupancy upon knockdown of SS18-SSX1 and KDM2B at 10,533 SS18-SSX/KDM2B co-occupied regions. (B) Box plots of fold change difference upon KDM2B.4395 in 10,533 SS18-SSX/KDM2B co-occupied regions and 451 KDM2B non-occupied regions, showing KDM2B knockdown primarily affects SS18-SSX occupancy at KDM2B bound regions. (C) Box plots of BRG1 ChIP signals at 8,488 BRG1-Loss regions and 3,424 BRG1-Gain regions in SSX.1274 in HS-SY-II cells. (D) Venn diagrams showing overlap between BRG1 losses and gains in SSX.1274 and KDM2B.4395 and respective gene ontology analysis using GREAT. (E) Box plots of fold change difference upon SSX.1274 and KDM2B.4395 at 4,091 ATAC-Seq high confidence gained peaks (F) Proposed model for KDM2B-dependent SS18-SSX activity. In synovial sarcoma binds unmethylated CpG islands of developmental genes. KDM2B-PRC1.1 promotes recruitment of the mutant SS18-SSX containing SWI/SNF complex by direct or indirect interaction with SS18-SSX leading to aberrant activation of developmental genes that would otherwise be repressed. Upon KDM2B inhibition SS18-SSX binding is reduced, allowing H3K27me3 gains at a sub-set of SS18-SSX targets, reduced gene accessibility and consequent down-regulation of expression of developmental proteins and TFs, possibly allowing re-establishment of normal differentiation programs. B., C. Unpaired t-test *** pvalue<0.0005

### DEFINITIONS

Administration: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time. As is known in the art, antibody therapy is commonly administered parenterally (e.g., by intravenous or subcutaneous injection).

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Animal: as used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, and antibody fragments. As used herein, the term “antibody agent” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. In some embodiments, the term encompasses stapled peptides. In some embodiments, the term encompasses one or more antibody-like binding peptidomimetics. In some embodiments, the term encompasses one or more antibody-like binding scaffold proteins. In come embodiments, the term encompasses monobodies or adnectins. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent is or comprises an antibody-drug conjugate.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Cancer: The terms “cancer”, “malignancy”, “neoplasm”, “tumor”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, hematopoietic cancers including leukemias, lymphomas (Hodgkins and non-Hodgkins), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Determine: Some methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Ex Vivo: As used herein refers to events that occur in or on tissue from a multi-cellular organism, such as a human and a non-human animal, in an external environment which resembles the natural conditions of the tissue with a minimum of alterations to the tissue itself.

Human: In some embodiments, a human is an embryo, a fetus, an infant, a child, a teenager, an adult, or a senior citizen.

Improve,” “increase” or “reduce: as used herein or grammatical equivalents thereof, indicate values that are relative to a baseline measurement, such as a measurement in the same individual or model system prior to initiation of a treatment or introduction of a test agent described herein, or a measurement in a control individual (or multiple control individuals) in the absence of the treatment described herein. In some embodiments, a “control individual” is an individual afflicted with the same form of disease or injury as an individual being treated.

Inhibitor: As used herein, the term “inhibitor” refers to an agent, condition, or event whose presence, level, degree, type, or form correlates with decreased level or activity of another agent (i.e., the inhibited agent, or target). In general, an inhibitor may be or include an agent of any chemical class including, for example, small molecules, polypeptides, nucleic acids, carbohydrates, lipids, metals, and/or any other entity, condition or event that shows the relevant inhibitory activity. In some embodiments, an inhibitor may be direct (in which case it exerts its influence directly upon its target, for example by binding to the target); in some embodiments, an inhibitor may be indirect (in which case it exerts its influence by interacting with and/or otherwise altering a regulator of the target, so that level and/or activity of the target is reduced).

In vitro: as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Nucleic acid: as used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3 -methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5 -propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

Polypeptide: as used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Protein: As used herein, the term “protein” refers to a polypeptide (i.e., a string of at least 3-5 amino acids linked to one another by peptide bonds). Proteins may include moieties other than amino acids (e.g., may be glycoproteins, proteoglycans, etc.) and/or may be otherwise processed or modified. In some embodiments “protein” can be a complete polypeptide as produced by and/or active in a cell (with or without a signal sequence); in some embodiments, a “protein” is or comprises a characteristic portion such as a polypeptide as produced by and/or active in a cell. In some embodiments, a protein includes more than one polypeptide chain. For example, polypeptide chains may be linked by one or more disulfide bonds or associated by other means. In some embodiments, proteins or polypeptides as described herein may contain L-amino acids, D-amino acids, or both, and/or may contain any of a variety of amino acid modifications or analogs known in the art. Useful modifications include, e.g., terminal acetylation, amidation, methylation, etc. In some embodiments, proteins or polypeptides may comprise natural amino acids, non-natural amino acids, synthetic amino acids, and/or combinations thereof. In some embodiments, proteins are or comprise antibodies, antibody polypeptides, antibody fragments, biologically active portions thereof, and/or characteristic portions thereof.

Reference: as used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating agent. In some embodiments, a small molecule is biologically active. In some embodiments, a small molecule is detectable (e.g., comprises at least one detectable moiety). In some embodiments, a small molecule is a therapeutic. Those of ordinary skill in the art, reading the present disclosure, will appreciate that certain small molecule compounds described herein may be provided and/or utilized in any of a variety of forms such as, for example, salt forms, protected forms, pro-drug forms, ester forms, isomeric forms (e.g., optical and/or structural isomers), isotopic forms, etc. In some embodiments, reference to a particular compound may relate to a specific form of that compound. In some embodiments, reference to a particular compound may relate to that compound in any form. In some embodiments, where a compound is one that exists or is found in nature, that compound may be provided and/or utilized in accordance in the present invention in a form different from that in which it exists or is found in nature. Those of ordinary skill in the art will appreciate that a compound preparation including a different level, amount, or ratio of one or more individual forms than a reference preparation or source (e.g., a natural source) of the compound may be considered to be a different form of the compound as described herein. Thus, in some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form; etc.

Subject: By “subject” is meant a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### DETAILED DESCRIPTION OF CERTAIN EMBODIMENTS

The compositions and methods of the present invention are useful for the treatment or diagnosis of cancer. In some embodiments, the compositions and methods of the present invention are useful for the treatment or diagnosis of sarcoma. In some embodiments, the compositions and methods of the present invention are useful for the treatment or diagnosis of synovial sarcoma.

Sarcoma

Malignant tumors of the connective tissues generally arising from cells of mesenchymal origin are called sarcomas. Sarcomas are divided into two main groups, bone sarcomas and soft tissue sarcomas. Sarcomas are further sub-classified based on the type of presumed cell of origin found in the tumor. Soft tissue sarcoma can occur in the muscles, fat, blood vessels, tendons, fibrous tissues and synovial tissues (tissues around joints). Approximately 15,000 new cases of sarcoma are diagnosed per year in the United States. Sarcomas represent about one percent of the 1.5 million new cancer diagnoses in the United States each year.

Sarcomas affect people of all ages. Approximately 50% of bone sarcomas and 20% of soft tissue sarcomas are diagnosed in people under the age of 35. Some sarcomas, such as leiomyosarcoma, chondrosarcoma, and gastrointestinal stromal tumor (GIST), are more common in adults than in children. Most high-grade bone sarcomas, including Ewing's sarcoma and osteosarcoma, are much more common in children and young adults.

Synovial sarcoma is an aggressive neoplasm that accounts for 10% to 20% of soft-tissue sarcomas in the adolescent and young adult population. Although it is typically diagnosed in young adults (median age 35), the age range is between 5 and 85 years. There is a slight male predeliction (M:F ratio 1.13); 70% of cases present in the extremities, and the most common pattern of metastatic spread is to the lung. The mainstay of treatment is wide surgical excision with adjuvant or neoadjuvant radiotherapy, which provides a good chance of cure for localized disease. However, the disease is prone to early and late recurrences, and 10-year disease-free survival remains on the order of 50%. Synovial sarcoma is moderately sensitive to cytotoxic chemotherapy with agents such as ifosfamide and anthracyclines.

Synovial sarcoma is uniquely characterized by the balanced chromosomal translocation t(X,18; p11,q11), demonstrable in virtually all cases, not found in any other human neoplasms. This translocation creates an in-frame fusion of the SS18 gene to SSX1 or SSX2, whereby all but the carboxy terminal (C-terminal) 8 amino acids of SS18 become fused to the C-terminal 78 amino acids of the SSX partner. An analogous translocation of SSX4 is detected in less than 1% of cases.

Multiple lines of evidence implicate SS18-SSX as the central genetic “driver” in this cancer: (i) its presence as the sole cytogenetic anomaly in up to a third of cases, (ii) the low frequency of additional mutations, (iii) its preservation in metastatic and advanced lesions, (iv) the death of synovial sarcoma cells upon SS18-SSX knockdown, and (v) its ability to induce tumors in conditional mouse models with appropriate histology, gene expression, and immunophenotype with 100% penetrance.

Although important for sarcomagenesis, SS18-SSX is devoid of a DNA binding domain and instead exerts its thought to exert its activity by interacting with chromatin binding proteins and modulators. The SS18-SSX protein product is able to bind transcriptional repressors, such as TLE1 and members of the polycomb repressive complex 2 (PRC2). Surprisingly, SS18-SSX but is also part of the activating chromatin remodeling SWI/SNF complex which play a role if transcriptional activation.

Soft tissue sarcomas are aggressive cancers afflicting children and young adults that rarely respond to conventional chemotherapy and are often lethal (Helman and Meltzer, 2003; Singer et al., 2000). Many present with recurrent chromosomal translocations that involve proteins thought to drive cancer by perturbing epigenetic mechanisms of gene regulation that, in principle, could be reversed. While the presence of such fusions further underscores the key relationship between cancer genetics and epigenetics during tumorigenesis, the mechanisms by which most chimeric oncoproteins drive oncogenesis remain poorly understood. Consequently, there are no therapeutic strategies to target their activity.

Synovial sarcoma is a paradigm of a gene fusion driven cancer, in which the defining event is the translocation t(X,18; p11, q11) that creates an in-frame fusion of the SS18 gene to SSX1, SSX2 or SSX4 genes (Clark et al., 1994; Ladanyi et al., 2002). SS18-SSX is present in virtually 100% of synovial sarcomas, being the only cytogenetic aberration in most of these tumors characterized by a very low frequency of additional genetic alterations (Nielsen et al., 2015). Accordingly, aberrant expression of the translocated gene product in the myoblast lineage of mice produces tumors that histologically and molecularly resemble the human disease (Haldar et al., 2007).

Unlike other soft tissues sarcomas translocations in which a transcription factor is thought to confer target specificity by binding DNA in a sequence specific manner (e.g. PAX3-FOXO1) (Mertens et al., 2016), SS18-SSX lacks a DNA binding domain and is thought to exert its activity by interacting with other chromatin regulators. The SSX family of transcriptional repressors proteins co-localize with polycomb group (PcG) proteins such as RING1B and BMI through unclear mechanisms (dos Santos et al., 2000; Soulez et al., 1999). By contrast, SS18 is a component of mammalian TrxG complexes (such as SWI/SNF) and, as a consequence, SS18-SSX interacts with components of the TrxG transcriptional activator proteins such as hBRM and BRG1 (Kadoch and Crabtree, 2013; Nagai et al., 2001; Soulez et al., 1999; Thaete et al., 1999).

While PcG proteins lead to chromatin compaction and gene repression, SWI/SNF complexes facilitate transcription by remodeling nucleosomes, thereby promoting gene activation by permitting increased access of transcription factors to their binding sites (Roberts and Orkin, 2004). It remains to be determined precisely how SS18-SSX affects the balance between transcriptional activation via SWI/SNF and PcG-associated gene repression. One study points to the ability of SS18-SSX to repress gene expression of tumor suppressor genes such as those encoded by the INK4a/ARF locus, a process depending on SS18-SSX ability to bridge ATF2 targets to TLE1 for recruitment of polycomb repressive complex 2 (PRC2) (Su et al., 2012). However another study suggests that SS18-SSX alters SWI/SNF composition and enhances its ability to oppose the H3K27me3 repressive mark at the Sox2 locus, leading to transcriptional activation (Kadoch and Crabtree, 2013). Efforts to confirm these mechanisms have been thwarted by an inability to identify direct targets of endogenous SS18-SSX. Thus, there is no comprehensive picture of how SS18-SSX binds chromatin, alters transcription, and drives oncogenic transformation. As SS18-SSX is not obviously druggable, such information is necessary for developing rational strategies to disrupt its action in synovial sarcoma.

Polycomb Repressive Complexes

Polycomb-group proteins (PcG) are a family of proteins that can remodel chromatin such that epigenetic silencing of genes takes place. There are two complexes within the PcG proteins: the Polycomb Repressive Complex 1 (PRC1) and Polycomb Repressive Complex 2 (PRC2). The PRC2 complex has histone methyltransferase activity and primarily trimethylates histone H3 on lysine 27 (i.e. H3K27me3), a mark of transcriptionally silent chromatin. PRC2 is required for initial targeting of genomic region (PRC Response Elements or PRE) to be silenced, while PRC1 is required for stabilizing this silencing and underlies cellular memory of silenced region after cellular differentiation. PRC1 also mono-ubiquitinates histone H2A on lysine 119 (H2AK119Ub1). These proteins are required for long term epigenetic silencing of chromatin and have an important role in stem cell differentiation and early embryonic development. PRC1 and PRC2 are present in all multicellular organisms. Additional non-canonical PRC complexes have been identified. Non-canonical PRC complexes include PRC1.1, PRC1.3, PRC 1.5, and PRC 1.6 In some embodiments, a PRC complex contains but is not limited to PCGF1, RYBP, BCOR, USP7, RING1A/B, KDM2B, PCGF2/4, PHC, SCML, and/or CBX.

PRC Inhibitory Agents

The present disclosure, among other things, contemplates use of an agent to inhibit one or more components of Polycomb Repressive Complexes. In some embodiments, the present disclosure provides PRC1.1 inhibitory agents. In some embodiments, a PRC1.1 inhibitory agent inhibits an individual component of PRC1.1. In some embodiments a PRC1.1 inhibitory agent is a KDM2B inhibitory agent. In some embodiments a PRC1.1 inhibitory agent is a BCOR inhibitory agent. In some embodiments a PRC1.1 inhibitory agent is a PCGF1 inhibitory agent. In some embodiments a PRC1.1 inhibitory agent is an agent that reduces interaction of PRC1.1 components with SS18-SSX. In some embodiments a PRC1.1 inhibitory agent is a polypeptide. In some embodiments a PRC1.1 inhibitory agent is a small molecule. In some embodiments a PRC1.1 inhibitory agent is a nucleic acid. In some embodiments a PRC1.1 inhibitory agent is an shRNA. In some embodiments a PRC1.1 inhibitory agent is an antibody agent.

In some embodiments a KDM2B inhibitory agent is an agent that inhibits demethylase activity. In some embodiments a KDM2B inhibitory agent is an agent that reduces interaction of KD2MB with SS18-SSX. In some embodiments a KDM2B inhibitory agent is a polypeptide. In some embodiments a KDM2B inhibitory agent is a small molecule. In some embodiments a KDM2B inhibitory agent is a nucleic acid. In some embodiments a KDM2B inhibitory agent is an shRNA. In some embodiments a KDM2B inhibitory agent is an antibody agent. In some embodiments a KDM2B inhibitory agent is PBIT (CAS 2514-30-9).

Administration

In some embodiments, an active agent for use in accordance with the present disclosure is formulated, dosed, and/or administered in a therapeutically effective amount using pharmaceutical compositions and dosing regimens that are consistent with good medical practice and appropriate for the relevant agent(s) and subject(s). In principle, therapeutic compositions can be administered by any appropriate method known in the art, including, without limitation, oral, mucosal, by-inhalation, topical, buccal, nasal, rectal, or parenteral (e.g. intravenous, infusion, intratumoral, intranodal, subcutaneous, intraperitoneal, intramuscular, intradermal, transdermal, or other kinds of administration involving physical breaching of a tissue of a subject and administration of the therapeutic composition through the breach in the tissue).

In some embodiments, a dosing regimen for a particular active agent may involve intermittent or continuous (e.g., by perfusion or other slow release system) administration, for example to achieve a particular desired pharmacokinetic profile or other pattern of exposure in one or more tissues or fluids of interest in the subject receiving therapy.

In some embodiments, different agents administered in combination may be administered via different routes of delivery and/or according to different schedules. Alternatively or additionally, in some embodiments, one or more doses of a first active agent is administered substantially simultaneously with, and in some embodiments via a common route and/or as part of a single composition with, one or more other active agents.

Factors to be considered when optimizing routes and/or dosing schedule for a given therapeutic regimen may include, for example, the particular indication being treated, the clinical condition of a subject (e.g., age, overall health, prior therapy received and/or response thereto) the site of delivery of the agent, the nature of the agent (e.g. an antibody or other polypeptide-based compound), the mode and/or route of administration of the agent, the presence or absence of combination therapy, and other factors known to medical practitioners. For example, in the treatment of cancer, relevant features of the indication being treated may include, for example, one or more of cancer type, stage, location.

In some embodiments, one or more features of a particular pharmaceutical composition and/or of a utilized dosing regimen may be modified over time (e.g., increasing or decreasing the amount of active agent in any individual dose, increasing or decreasing time intervals between doses), for example in order to optimize a desired therapeutic effect or response.

In general, type, amount, and frequency of dosing of active agents in accordance with the present invention are governed by safety and efficacy requirements that apply when one or more relevant agent(s) is/are administered to a mammal, preferably a human. In general, such features of dosing are selected to provide a particular, and typically detectable, therapeutic response as compared to what is observed absent therapy.

In the context of the present invention, an exemplary desirable therapeutic response may involve, but is not limited to, inhibition of and/or decreased tumor growth, tumor size, metastasis, one or more of the symptoms and side effects that are associated with a tumor, as well as increased apoptosis of cancer cells, therapeutically relevant decrease or increase of one or more cell marker or circulating markers, cell cycle arrest, differentiation into a more mesenchymal like state. Such criteria can be readily assessed by any of a variety of immunological, cytological, and other methods that are disclosed in the literature.

In some embodiments, an effective dose (and/or a unit dose) of an active agent, may be at least about 0.01 μg/kg body weight, at least about 0.05 μg/kg body weight; at least about 0.1 μg/kg body weight, at least about 1 μg/kg body weight, at least about 2.5 μg/kg body weight, at least about 5 μg/kg body weight, and not more than about 100 μg/kg body weight. It will be understood by one of skill in the art that in some embodiments such guidelines may be adjusted for the molecular weight of the active agent. The dosage may also be varied for route of administration, the cycle of treatment, or consequently to dose escalation protocol that can be used to determine the maximum tolerated dose and dose limiting toxicity (if any) in connection to the administration of a PRC1.1 inhibitory agent and/or an additional therapeutic agent at increasing doses. Consequently, the relative amounts of the each agent within a pharmaceutical composition may also vary, for example, each composition may comprise between 0.001% and 100% (w/w) of the corresponding agent.

In some embodiments, toxicity and/or therapeutic efficacy PRC1.1 inhibitory agents can be determined by standard pharmaceutical procedures in cell cultures or experimental animals, e.g., for determining the maximum tolerated dose (MTD) and the ED₅₀ (effective dose for 50% maximal response). Typically, the dose ratio between toxic and therapeutic effects is the therapeutic index; in some embodiments, this ratio can be expressed as the ratio between MTD and ED₅₀. Data obtained from such cell culture assays and animal studies can be used in formulating a range of dosage for use in humans.

One of skill in the art can select from a variety of administration regimens and will understand that an effective amount of a particular PRC1.1 inhibitory agent may be dependent on the subject being treated, on the subject's weight, the severity of the affliction, the manner of administration and/or the judgment of the prescribing physician.

Combination Therapy

In some embodiments a PRC1.1 inhibitory agent (e.g., a KDM2B inhibitory agent) can be used in combination with another therapeutic agent or treatment for cancer (e.g., synovial sarcoma). In some embodiments, a PRC1.1 inhibitory agent, or a pharmaceutical composition comprising a PRC1.1 inhibitory agent as described herein can optionally contain, and/or be administered in combination with, one or more additional therapeutic agents, such as a cancer therapeutic agent, e.g., a chemotherapeutic agent or a biological agent. An additional agent can be, for example, a therapeutic agent that is art-recognized as being useful to treat the disease or condition being treated by the PRC1.1 inhibitory agent, e.g., an anti-cancer agent, or an agent that ameliorates a symptom associated with the disease or condition being treated. The additional agent also can be an agent that imparts a beneficial attribute to the therapeutic composition (e.g., an agent that affects the viscosity of the composition). For example, in some embodiments, a PRC1.1 inhibitory agent is administered to a subject who has received, is receiving, and/or will receive therapy with another therapeutic agent or modality (e.g., with a chemotherapeutic agent, surgery, radiation, or a combination thereof).

Some embodiments of combination therapy modalities provided by the present disclosure provide, for example, administration of a PRC1.1 inhibitory agent and additional agent(s) in a single pharmaceutical formulation. Some embodiments provide administration of a PRC1.1 inhibitory agent and administration of an additional therapeutic agent in separate pharmaceutical formulations.

Examples of chemotherapeutic agents that can be used in combination with a PRC1.1 inhibitory agent described herein include platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

Examples of biological agents that can be used in the compositions and methods described herein include monoclonal antibodies (e.g., rituximab, cetuximab, panetumumab, tositumomab, trastuzumab, alemtuzumab, gemtuzumab ozogamicin, bevacizumab, catumaxomab, denosumab, obinutuzumab, ofatumumab, ramucirumab, pertuzumab, ipilimumab, nivolumab, nimotuzumab, lambrolizumab, pidilizumab, siltuximab, BMS-936559, RG7446/MPDL3280A, MEDI4736, tremelimumab, or others known in the art), enzymes (e.g., L-asparaginase), cytokines (e.g., interferons and interleukins), growth factors (e.g., colony stimulating factors and erythropoietin), cancer vaccines, gene therapy vectors, or any combination thereof.

In some embodiments, a PRC1.1 inhibitory agent is administered to a subject in need thereof in combination with another agent for the treatment of cancer, either in the same or in different pharmaceutical compositions. In some embodiments, the additional agent is an anticancer agent. In certain embodiments, an additional anticancer agent is selected from the group consisting of chemotherapeutics (such as 2CdA, 5-FU, 6-Mercaptopurine, 6-TG, Abraxane™, Accutane®, Actinomycin-D, Adriamycin®, Alimta®, all-trans retinoic acid, amethopterin, Ara-C, Azacitadine, BCNU, Blenoxane®, Camptosar®, CeeNU®, Clofarabine, Clolar™, Cytoxan®, daunorubicin hydrochloride, DaunoXome®, Dacogen®, DIC, Doxil®, Ellence®, Eloxatin®, Emcyt®, etoposide phosphate, Fludara®, FUDR®, Gemzar®, Gleevec®, hexamethylmelamine, Hycamtin®, Hydrea®, Idamycin®, Ifex®, ixabepilone, Ixempra®, L-asparaginase, Leukeran®, liposomal Ara-C, L-PAM, Lysodren, Matulane®, mithracin, Mitomycin-C, Myleran®, Navelbine®, Neutrexin®, nilotinib, Nipent®, Nitrogen Mustard, Novantrone®, Oncaspar®, Panretin®, Paraplatin®, Platinol®, prolifeprospan 20 with carmustine implant, Sandostatin®, Targretin®, Tasigna®, Taxotere®, Temodar®, TESPA, Trisenox®, Valstar®, Velban®, Vidaza™, vincristine sulfate, VM 26, Xeloda® and Zanosar®); biologics (such as Alpha Interferon, Bacillus Calmette-Guerin, Bexxar®, Campath®, Ergamisol®, Erlotinib, Herceptin®, Interleukin-2, Iressa®, lenalidomide, Mylotarg®, Ontak®, Pegasys®, Revlimid®, Rituxan®, Tarceva™, Thalomid®, Velcade® and Zevalin™); small molecules (such as Tykerb®); corticosteroids (such as dexamethasone sodium phosphate, DeltaSone® and Delta-Cortef®); hormonal therapies (such as Arimidex®, Aromasin®, Casodex®, Cytadren®, Eligard®, Eulexin®, Evista®, Faslodex®, Femara®, Halotestin®, Megace®, Nilandron®, Nolvadex®, Plenaxis™ and Zoladex®); and radiopharmaceuticals (such as Iodotope®, Metastron®, Phosphocol® and Samarium SM-153).

In some embodiments, a PRC1.1 inhibitory agent is administered to a subject in need thereof in combination with another agent for the treatment of synovial sarcoma. In some embodiments, a PRC1.1 inhibitory agent is administered to a subject in need thereof in combination with ifosfamide, and or other agents described herein, and mesna. In some embodiments, a PRC1.1 inhibitory agent is administered to a subject in need thereof in combination with MAID therapy (i.e. administered in combination with mesna, adriamycin [doxorubicin], ifosfamide, and dacarbazine).

The additional agents that can be used in combination with a PRC1.1 inhibitory agent as set forth above are for illustrative purposes and not intended to be limiting. The combinations embraced by this disclosure, include, without limitation, one or more PRC1.1 inhibitory agents as provided herein or otherwise known in the art, and at least one additional agent selected from the lists above or otherwise provided herein. The PRC1.1 inhibitory agent can also be used in combination with one or with more than one additional agent, e.g., with two, three, four, five, or six, or more, additional agents.

In some embodiments, treatment methods described herein are performed on subjects for which other treatments of the medical condition have failed or have had less success in treatment through other means, e.g., in subjects having a cancer refractory to standard-of-care treatment. Additionally, the treatment methods described herein can be performed in conjunction with one or more additional treatments of the medical condition, e.g., in addition to or in combination with standard-of-care treatment. For instance, the method can comprise administering a cancer regimen, e.g., nonmyeloablative chemotherapy, surgery, hormone therapy, and/or radiation, prior to, substantially simultaneously with, or after the administration of a PRC1.1 inhibitory agent described herein, or composition thereof. In certain embodiments, a subject to which a PRC1.1 inhibitory agent described herein is administered can also be treated with antibiotics and/or one or more additional pharmaceutical agents.

### EXEMPLIFICATION

### Example 1

The mSWI/SNF complex is frequently mutated in cancer and neurodevelopmental disorders. In synovial sarcoma (SS), a sub-type of soft-tissue sarcoma that arises most frequently in adolescents and young adults, the defining genetic event is the translocation of the mSWI/SNF component SS18 on chromosome 18q11 to either the SSX1 and SSX2 genes located on chromosome Xp11. The resulting SS18-SSX fusion oncoprotein lacks a DNA binding domain, but is thought to exert its function via interaction with transcription factors and chromatin remodelers. No strategies exist to directly inhibit SS18-SSX however, since epigenetic dysregulation is central to the biology of synovial sarcoma, tumor cells driven by SS18-SSX may acquire epigenetic dependencies critical for tumor maintenance.

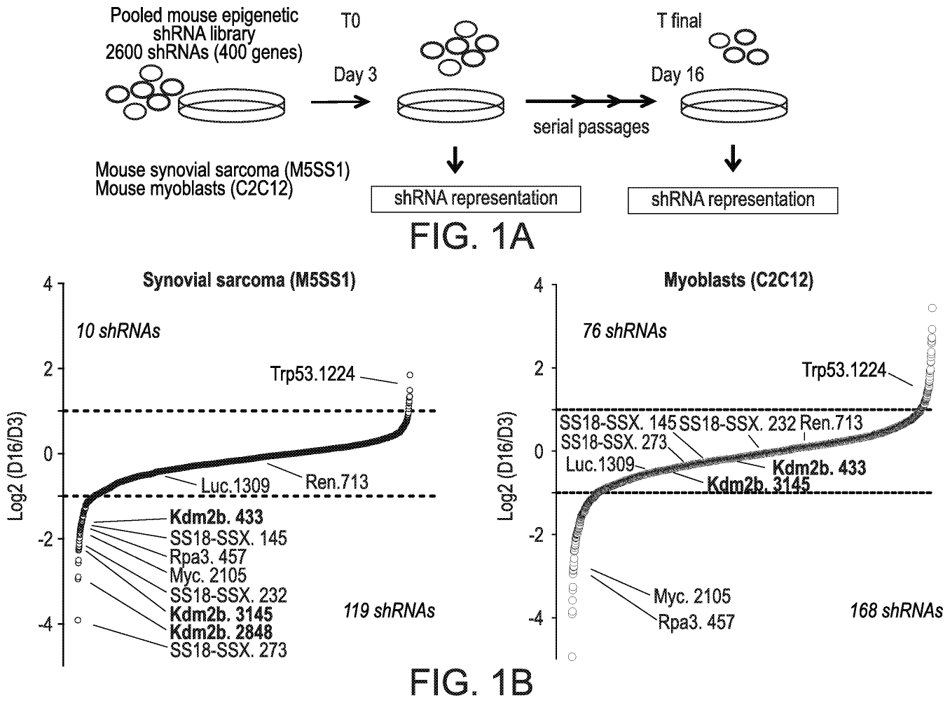

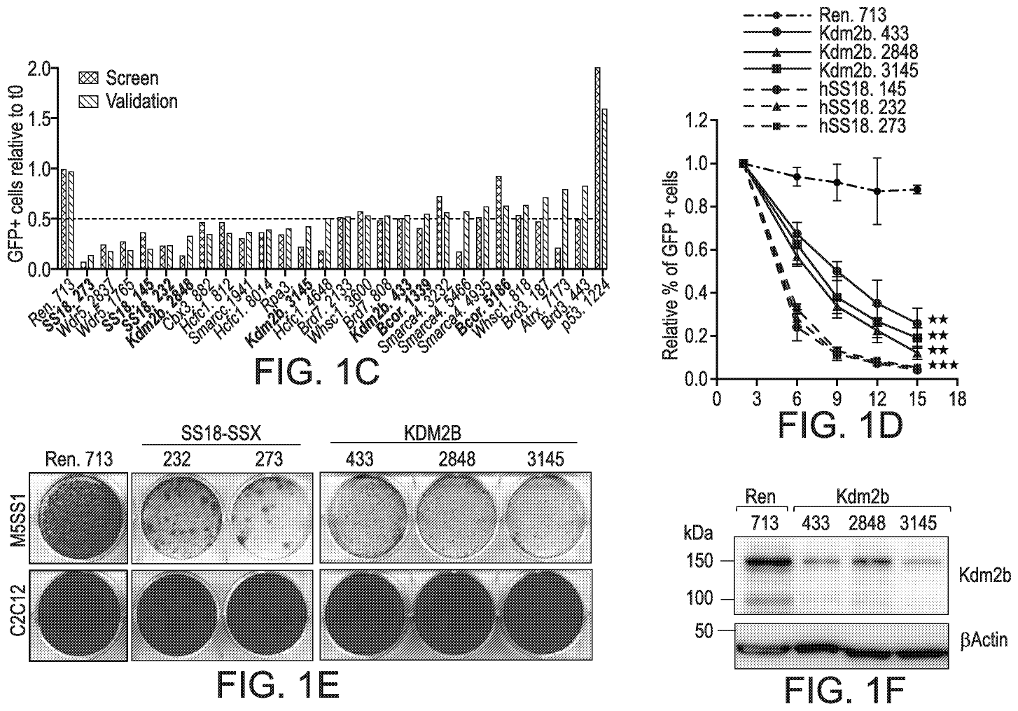

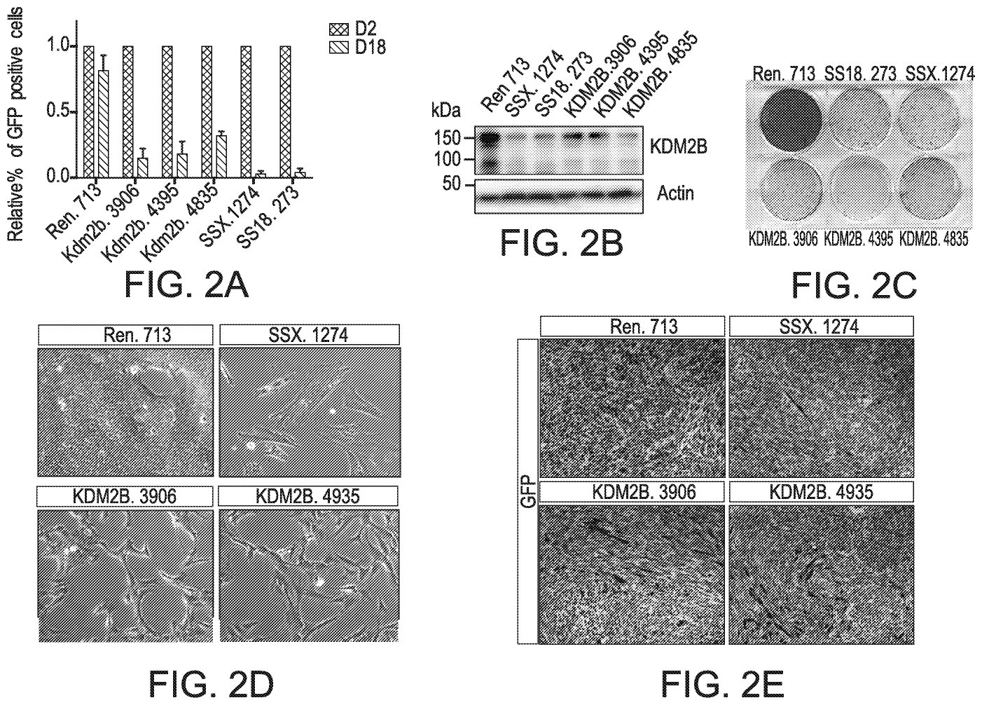

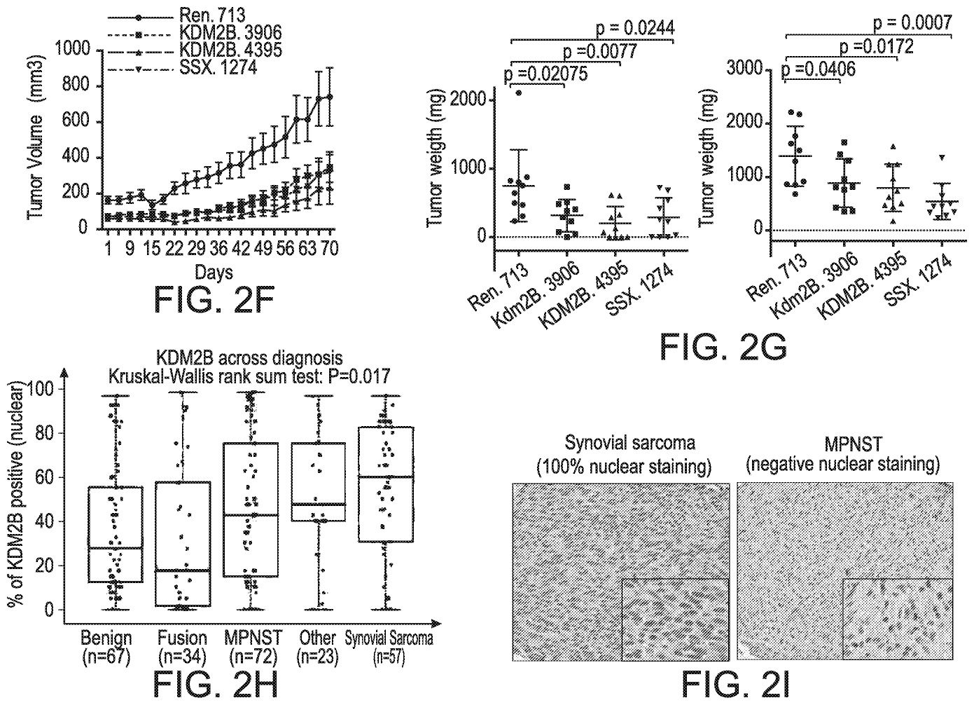

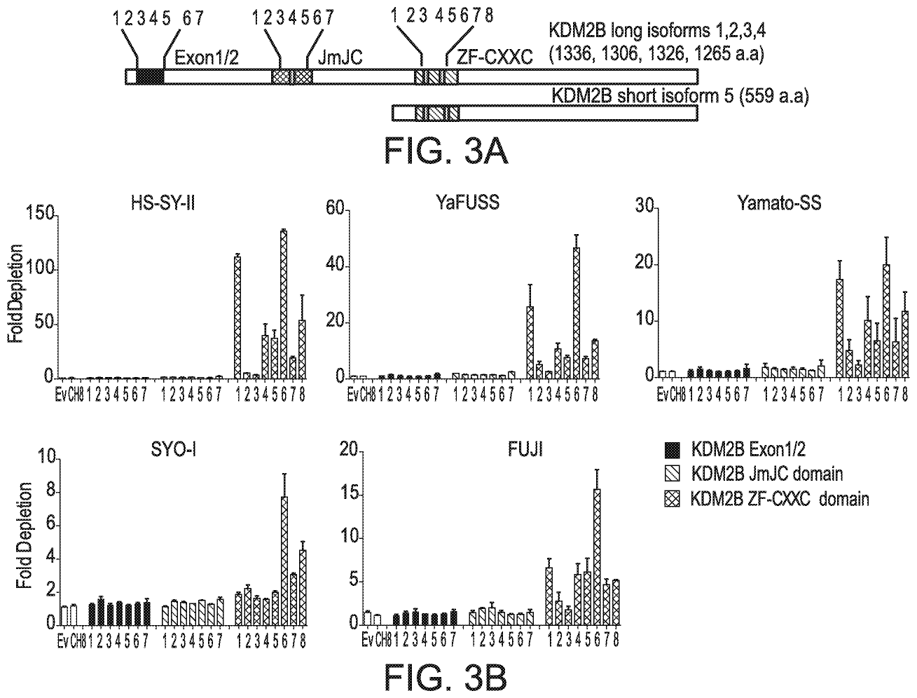

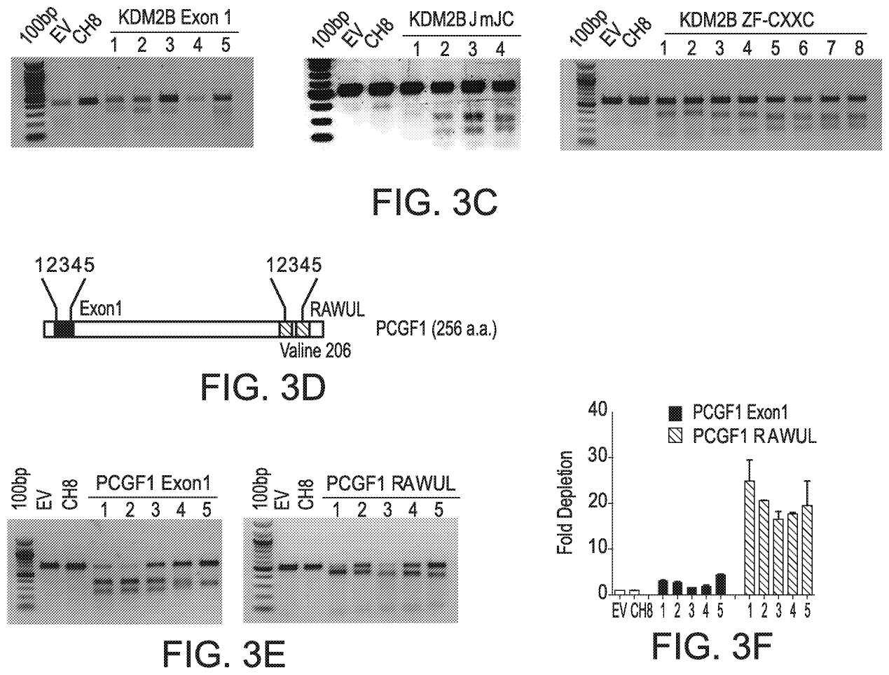

To identify such epigenetic vulnerabilities, we screened a mouse synovial sarcoma cell line derived from a mouse model in which the human SS18-SSX2 fusion oncogene is expressed in the myogenic progenitor compartment, described in Haldar et al. Cancer Cell 2007; 11:375-88 which is incorporated herein in its entirety, using an shRNA library targeting epigenetic modulators (2400 shRNAs). C2C12 mouse myoblasts were screened in parallel as an untransformed control. See FIG. 1. Consistent with a central role in synovial sarcoma maintenance, three shRNAs targeting SS18-SSX were among the top depletion hits. Among the 119 shRNAs depleted in the sarcoma cell line three independent shRNAs targeting Kdm2b specifically scored in sarcoma cells. These results were validated in human patient-derived synovial sarcoma cell lines. See FIG. 2. Besides affecting proliferation in vitro, KDM2B inhibited tumor growth in vivo in synovial sarcoma xenografts. KDM2B has an established role as an H3K36me2 demethylase, however it was recently described to be a component of the non-canonical polycomb repressive complex 1 (PRC1.1) where it mediates its recruitment to unmethylated CpG islands (CGIs) independently of PRC2. By targeting CRISPR-Cas9 mutagenesis to exons encoding functional protein domains in KDM2B, we identified the zinc finger domain (ZF-CxxC), which recognizes unmethylated CGIs, as required for sarcoma maintenance. Consistent with a role for SWI/SNF in opposing PRC2 activity, genome-wide profiling of H3K27me3 and KDM2B occupancy reveled that SS18-SSX knockdown results in gains of the H3K27me3 repressive mark at a subset of KDM2B targets. These targets are enriched in developmental transcription factors involved in neural specification. Importantly, RNA-seq data comprising 265 patient samples of various sarcoma subtypes (TCGA), revealed that these genes are among the top 100 genes overexpressed in SS when compared to other sarcomas, and their expression is inhibited as a result of SS18-SSX or KDM2B knockdown. To determine if these are direct targets of the fusion oncoprotein, we engineered a patient derived SS line using CRISPR/Cas9 based homologous recombination to tag endogenous SS18-SSX. Chromatin immunoprecipitation sequencing (Chip-seq) confirmed that SS18-SSX binds to KDM2B-bound CGIs at the promoters of these developmental genes and that KDM2B ablation results in decreased SS18-SSX binding. Finally, co-immunoprecipitation studies showed that KDM2B, and members of the PRC1.1, interact with the SS18-SSX fusion protein, further suggesting that this complex is required for SS18-SSX to maintain an epigenetic landscape that promotes proliferation and deregulation of normal differentiation programs in mesenchymal progenitors.

Our approach allowed the identification of a specific epigenetic dependency in synovial sarcoma, uncovering the KDM2B-PRC1 complex as new therapeutic target. Furthermore we identified for the first time genome-wide target genes of the SS18-SSX oncoprotein, shedding light into the biological mechanisms underlying this disease. In doing so we uncovered a yet unknown and unexpected requirement for the non-canonical PRC1.1 complex in mediating the opposition between polycomb repression and mSWI/SNF activity, a concept with potential important implications in development and disease.

### Example 2

### Role of KDM2B-PRC1.1 Interaction with SS18-SSX Oncoprotein in Synovial Sarcoma

Efforts to elucidate how cancer-causing mutations sustain the transformed state often reveal novel mechanisms of oncogenesis and rational strategies for therapeutic intervention. As most synovial sarcomas harbor few, if any, genetic alterations besides an SS18-SSX fusion, we reasoned that a global deregulation of epigenetic networks might be essential for the initiation and maintenance of this disease. Therefore, a negative selection small hairpin RNA (shRNA) screen was performed to identify specific epigenetic vulnerabilities created by the SS18-SSX oncoprotein. This approach identified a critical role for KDM2B and members of the non-canonical polycomb repressive complex 1 (PRC1.1) in sustaining synovial sarcoma. By tagging the endogenous SS18-SSX chimera in human synovial sarcoma cells and analyzing target genes, it was found that KDM2B recruits SS18-SSX and the SWI/SNF complex to unmethylated CpG islands, allowing the fusion to activate genes that would otherwise be repressed and producing the hallmark transcriptional profile of this disease. Consequently, KDM2B depletion suppresses oncogenesis by triggering cell cycle arrest and the differentiation of synovial sarcoma cells into a more mesenchymal like state. These results validate KDM2B as a therapeutic target for synovial sarcoma and reveal a novel mechanism by which an oncogenic fusion exploits the antagonism between SWI-SNF and Polycomb complexes to sustain cellular transformation.

An shRNA Screen Identifies KDM2B as a Specific Vulnerability of SS18-SSX Driven Tumors.

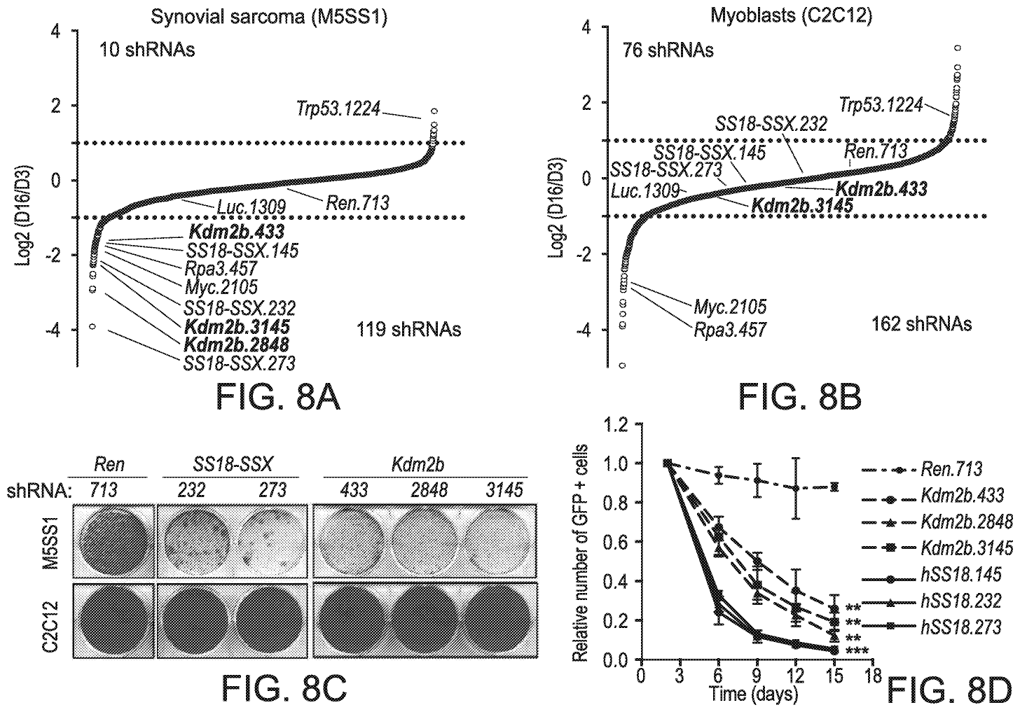

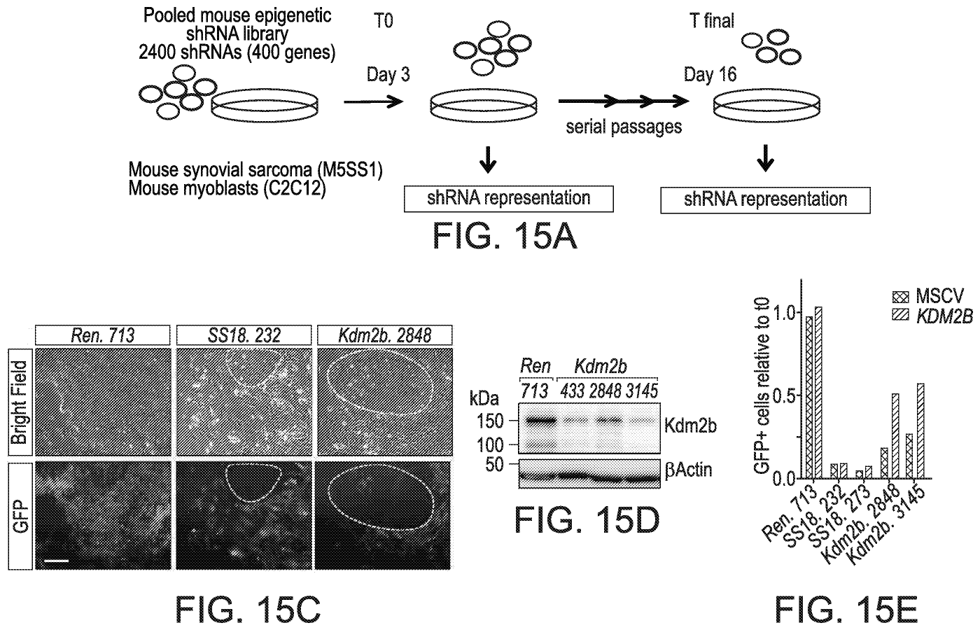

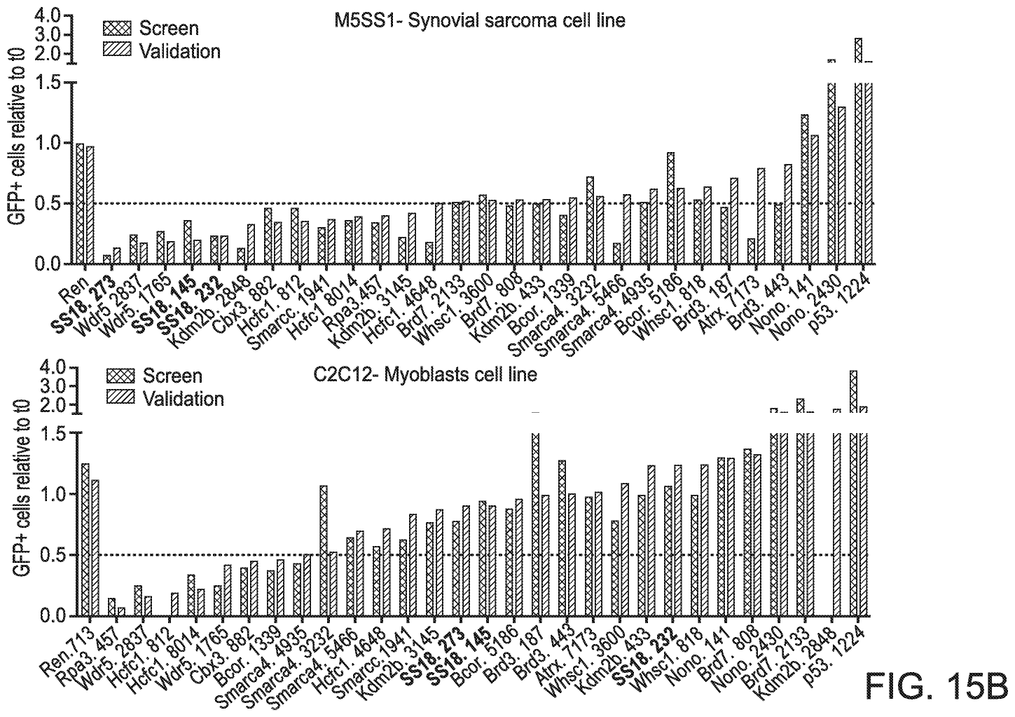

To interrogate the epigenetic mechanisms that sustain synovial sarcoma, a pool-based shRNA screen was performed to identify chromatin regulators whose inhibition selectively suppressed the proliferation of synovial sarcoma cells. A library consisting of ˜2400 GFP-coupled shRNAs targeting 400 chromatin regulators was transformed into a synovial sarcoma cell line (M5SS1) derived from a murine sarcoma induced by expression of the human SS18-SSX2 cDNA in the a mesenchymal progenitor lineage (Haldar et al., 2007). As a control, the same library was introduced into untransformed C2C12 mouse myoblasts (FIG. 15A). Changes in library representation after 16 days of continuous passaging in culture were monitored using deep sequencing of shRNA guide strands amplified from genomic DNA. As expected, shRNAs targeting the positive control genes Rpa3 (a replication enzyme) and c-Myc were depleted upon cell passaging in both cell lines, whereas an shRNA targeting the Trp53 tumor suppressor was substantially enriched (FIGS. 8A and 8B). Notably, three shRNAs targeting the SS18-SSX fusion oncogene were strongly depleted in M5SS1 but not in C2C12 cells (FIGS. 8A and 8B), confirming that murine synovial sarcoma cells depend on continuous SS18-SSX expression.

To identify additional shRNAs that mimicked those targeting SS18-SSX, the sequencing data was analyzed using specific scoring criteria (see Methods). Some shRNAs were depleted in both M5SS1 and C2C12 cells, including shRNAs targeting the SWI/SNF component Smarca4 (FIG. 15B). By contrast, others exhibited mild or no depletion in either cell line, including those targeting PRC2 subunits Ezh2/1 and Suz12. shRNAs that were preferentially depleted in M5SS1 cells included those targeting Brd7 and Brd3 with shRNAs targeting Kdm2b being the most potently and consistently depleted (FIGS. 8A and 15B). All three scoring Kdm2b shRNAs potently suppressed KDM2B protein expression and selectively impaired the proliferation of synovial sarcoma cells in competition and clonogenic survival assays (FIGS. 8C and 8D and FIGS. 15C and 15D). Confirming an on-target effect, a non-targetable Kdm2b cDNA restored the proliferation of synovial sarcoma cells expressing Kdm2b shRNAs (FIG. 15E). Thus, KDM2B is required for sustained proliferation of murine synovial sarcoma cells.

KDM2B Is Important for the Maintenance of Human Synovial Sarcoma Cells

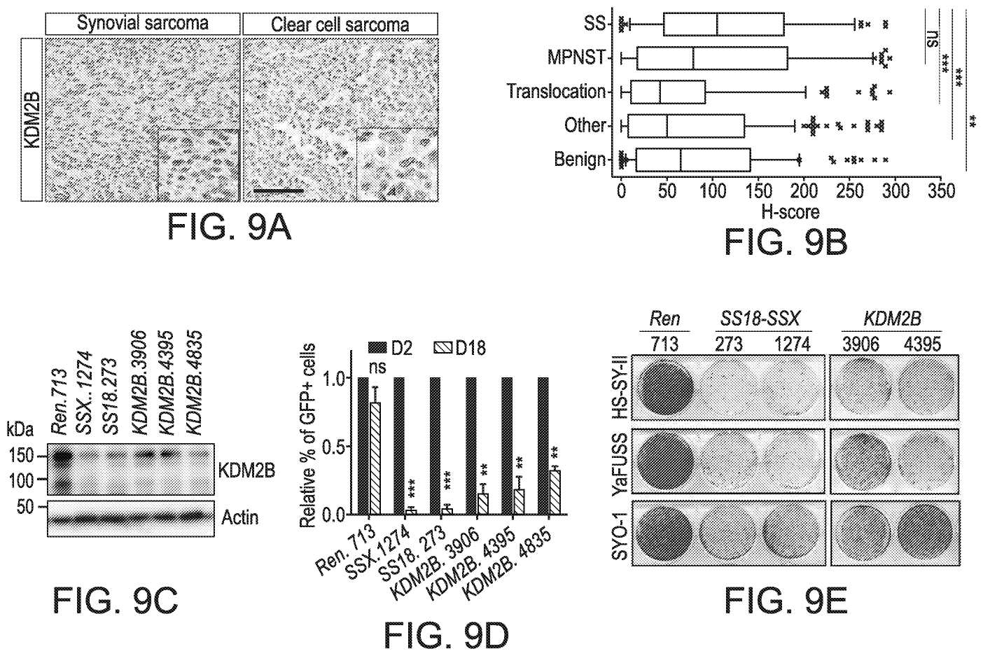

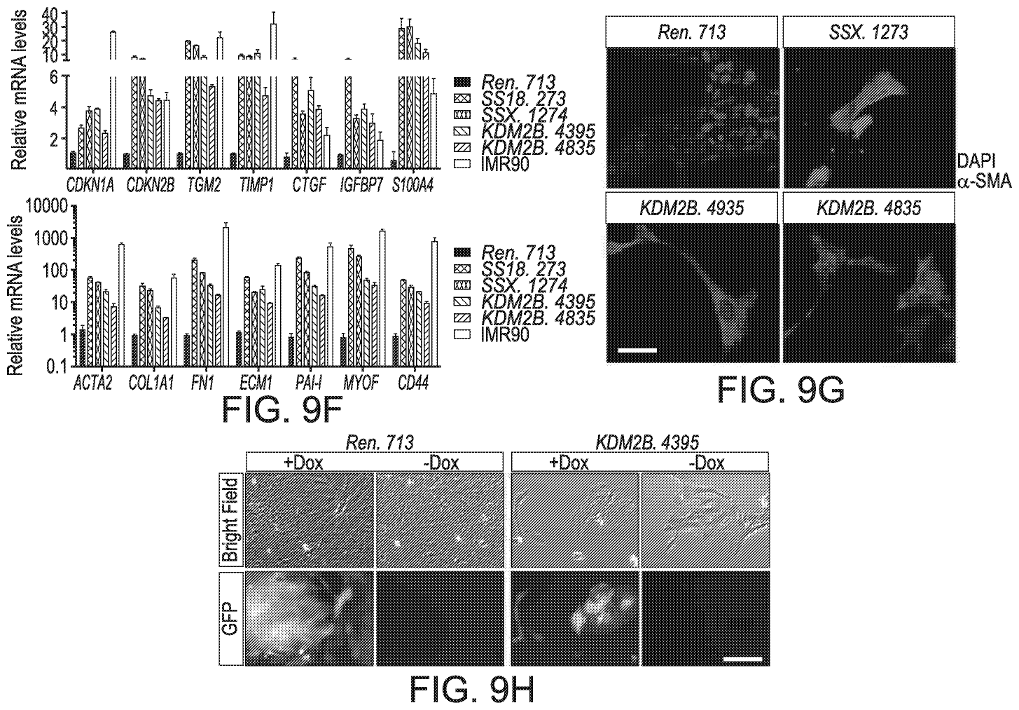

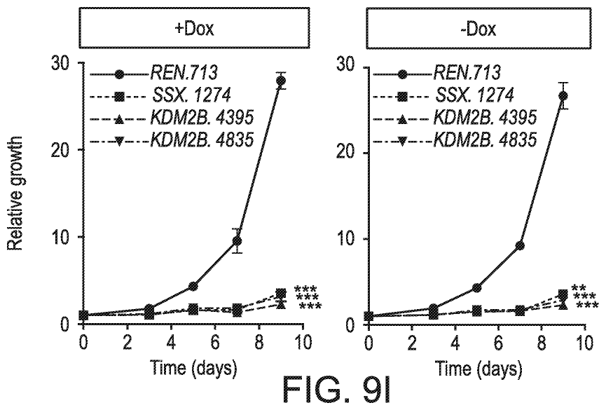

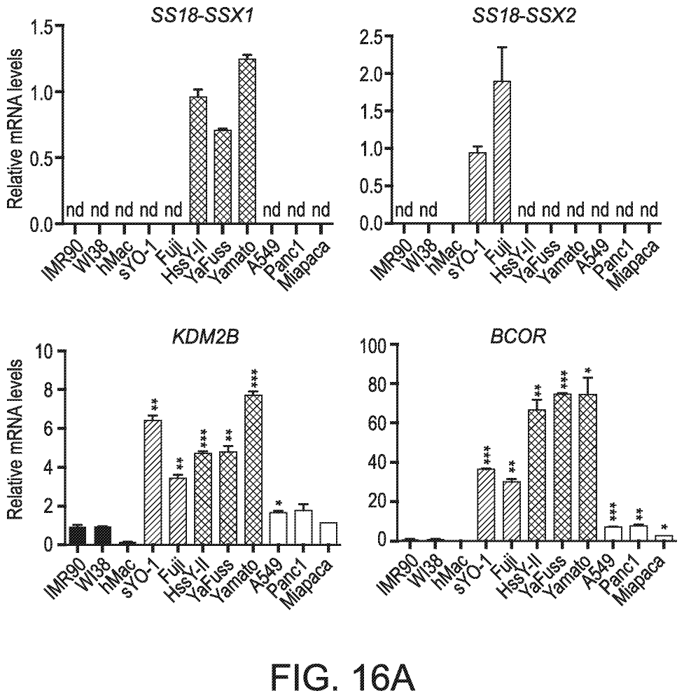

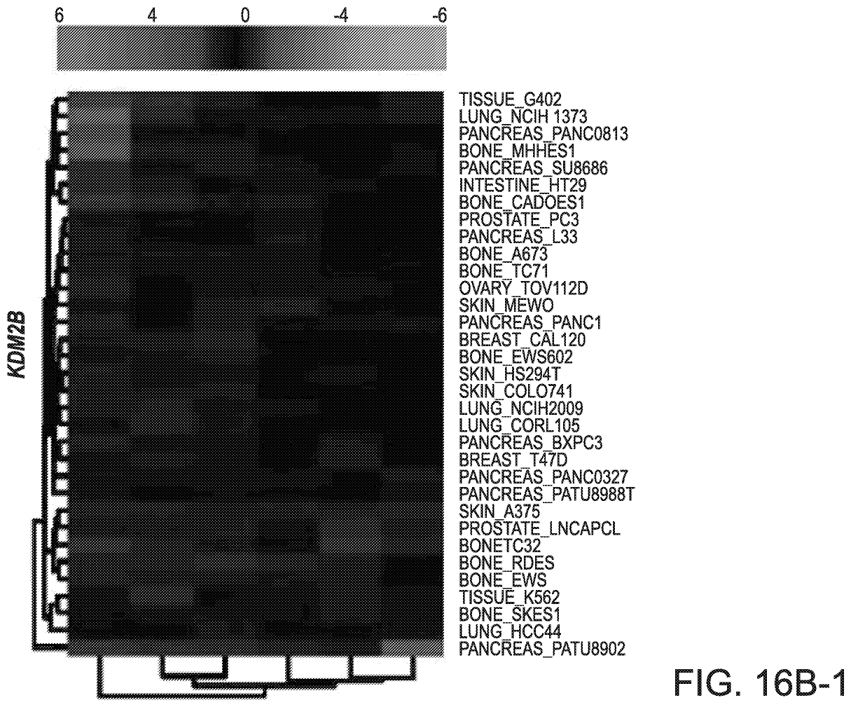

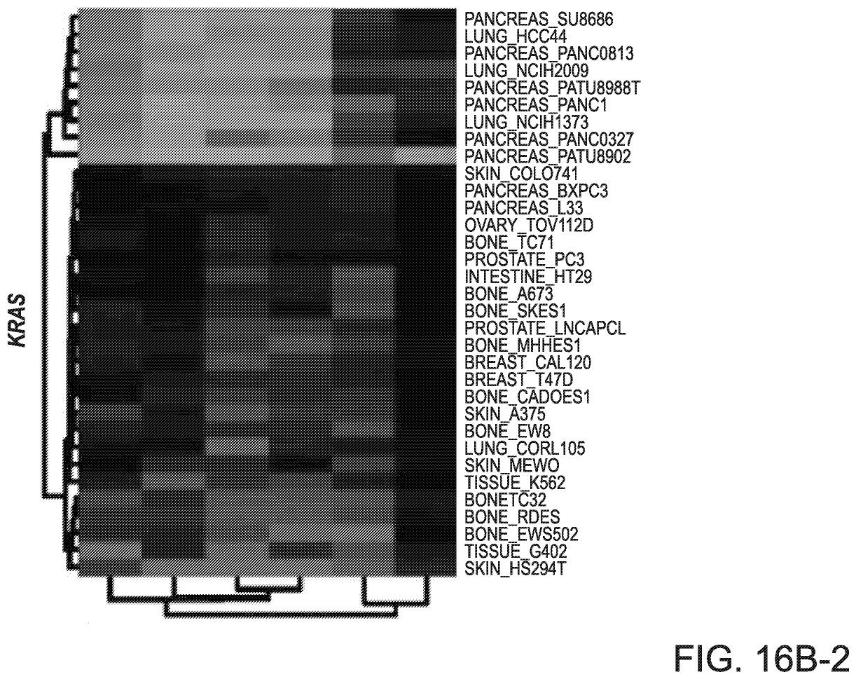

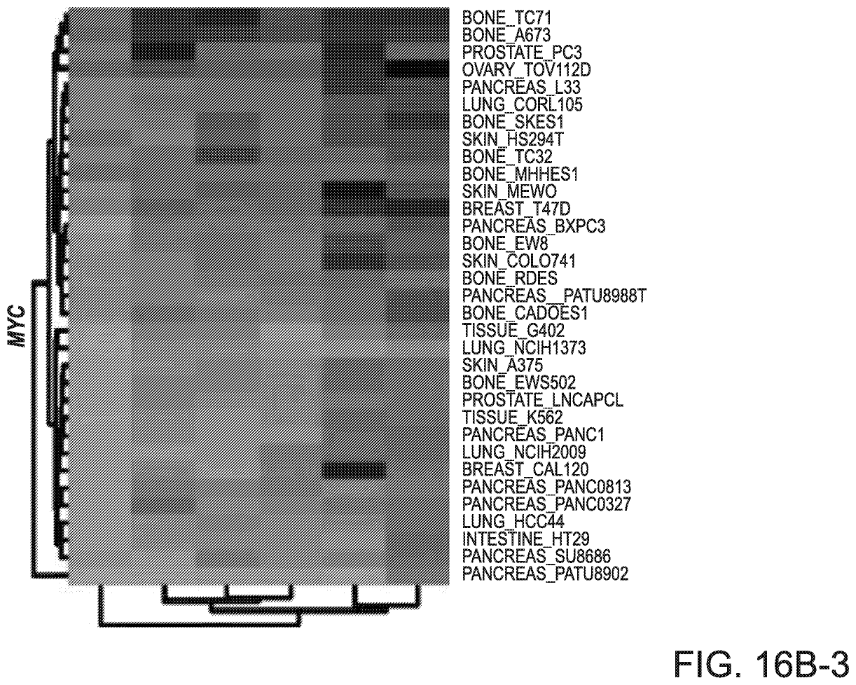

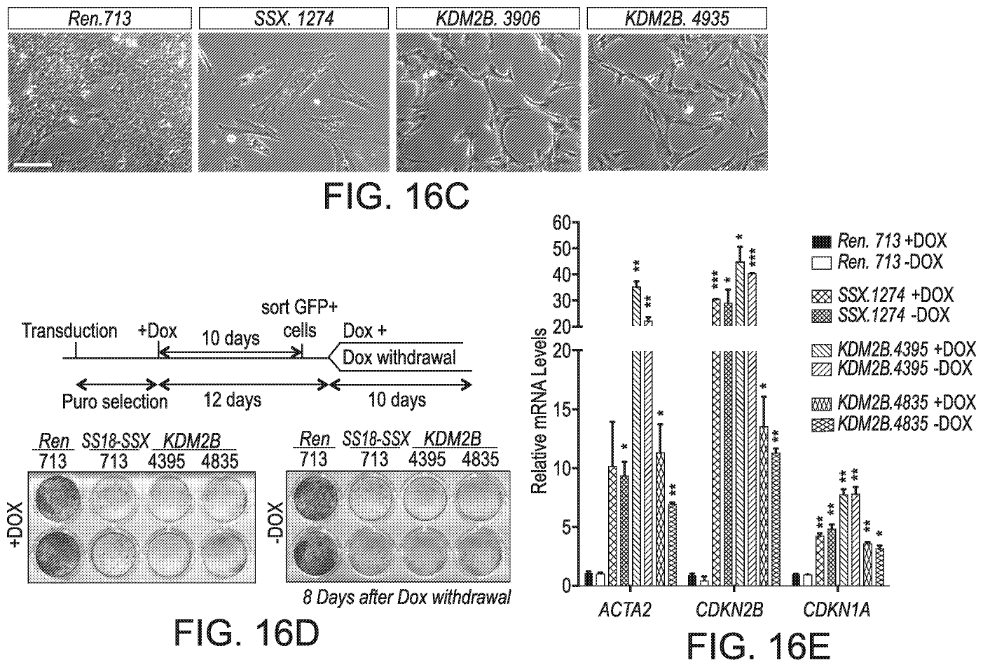

Next, KDM2B protein expression across different human sarcoma types was examined and it was tested whether KDM2B was required for the proliferation and tumorigenic potential of human synovial sarcoma cells. Immunohistochemistry of a large panel of human sarcomas revealed that synovial sarcoma cells express high levels of KDM2B protein (FIGS. 9A and 9B). Similarly, KDM2B mRNA levels were higher in synovial sarcoma cell lines than normal fibroblasts or other cancer cell lines (FIG. 16A) and, accordingly, analysis of publicly available functional genomics data confirmed that KDM2B is not universally required for cell proliferation (FIG. 16B)(Aguirre et al., 2016). RNAi-mediated suppression of KDM2B in a panel of human synovial sarcoma cells triggered proliferative arrest and the acquisition of a fibroblast-like morphology in a manner that was remarkably similar to knockdown of SS18-SSX using either SS18 or SSX1/2 shRNAs (FIGS. 9C-9E and FIG. 16C). Accordingly, cells subjected to SS18-SSX or KDM2B inhibition upregulated genes indicative of mesenchymal differentiation, including those encoding certain extracellular matrix proteins and secreted proteins highly expressed in human fibroblasts, such as COL1A1, SERPINE1 (PAI-1), and ACTA2 (α-SMA) and the cell cycle inhibitors CDKN1A and CDKN2B (FIGS. 9F and 9G). This cell fate transition was irreversible: cells harboring GFP-coupled, doxycycline (DOX)-inducible shRNAs targeting either SS18-SSX or KDM2B showed proliferative arrest and mesenchymal differentiation upon DOX addition, which was maintained upon shRNA silencing following DOX withdrawal (FIGS. 9H, 9I and FIGS. 16D-16E). Apparently, SS18-SSX or KDM2B inhibition disrupts synovial sarcoma maintenance by triggering proliferative arrest and differentiation into a more mesenchymal cell fate.

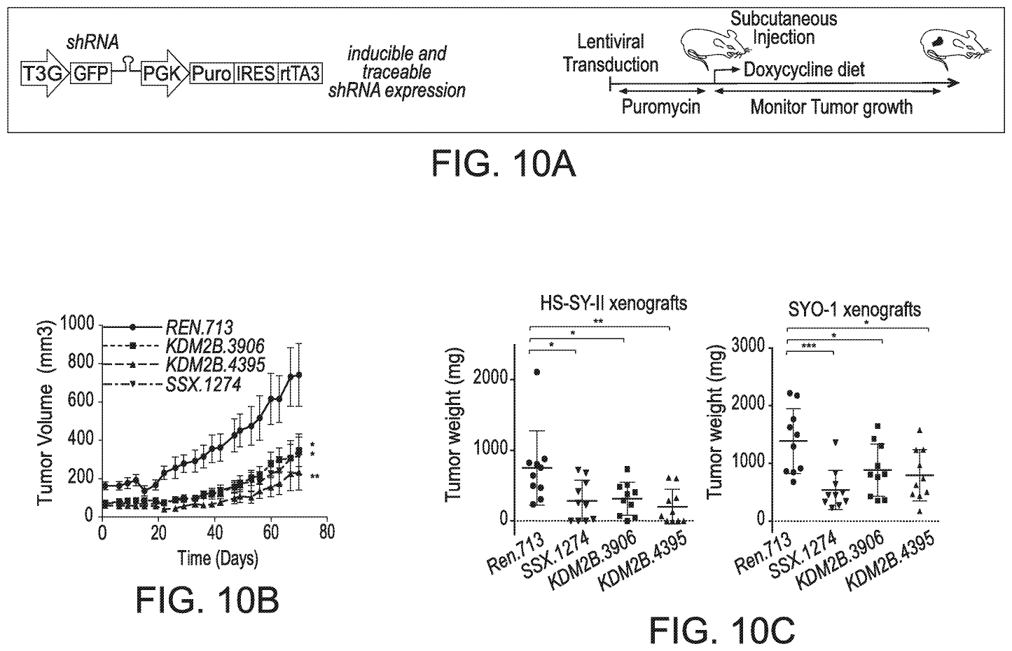

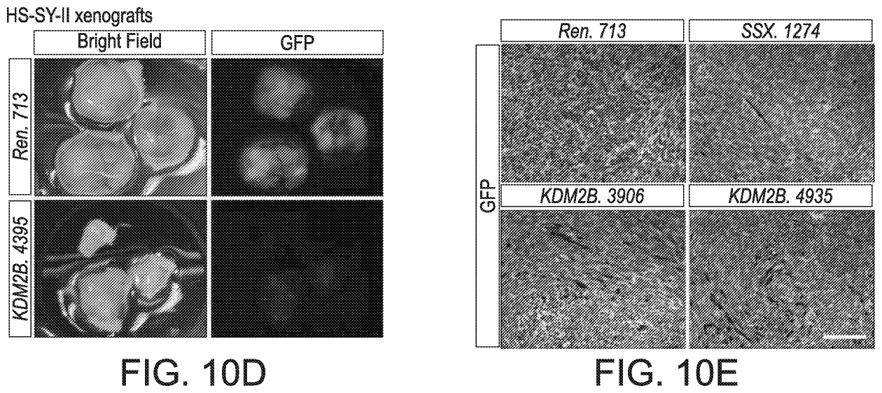

The impact of SS18-SSX and KDM2B inhibition on tumor growth in vivo was also examined. HS-SY-II and SYO-1 synovial sarcoma cell lines expressing the inducible GFP-coupled shRNAs described above were transplanted into immunocompromised mice that were fed a DOX-containing diet to activate shRNA expression (FIG. 10A). Tumor xenografts expressing KDM2B shRNAs displayed markedly impaired tumor growth, closely mimicking the effects seen by RNAi-mediated downregulation of SS18-SSX (FIGS. 10B and 10C). Importantly, and in contrast to tumors harboring control shRNAs, tumors arising from cells transduced with SS18-SSX or KDM2B shRNAs were composed predominantly of GFP-negative cells that had lost or silenced the shRNA (FIGS. 10D and 10E and FIG. 16F). Therefore, human synovial sarcoma cells also require KDM2B for tumor maintenance in vivo.

The DNA-Binding Domain of KDM2B and PRC1.1 is Important for Synovial Sarcoma Proliferation

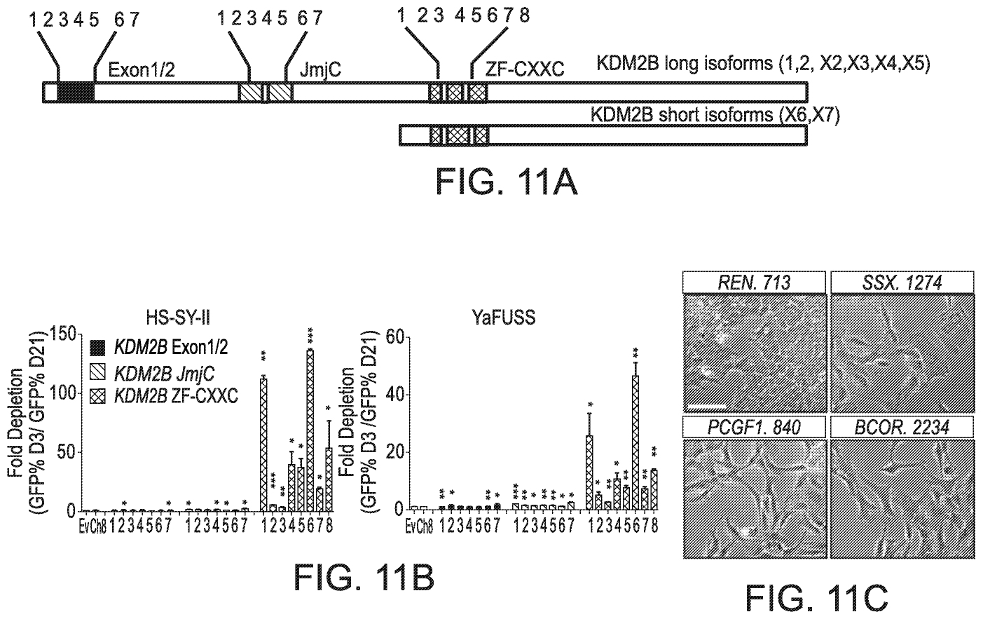

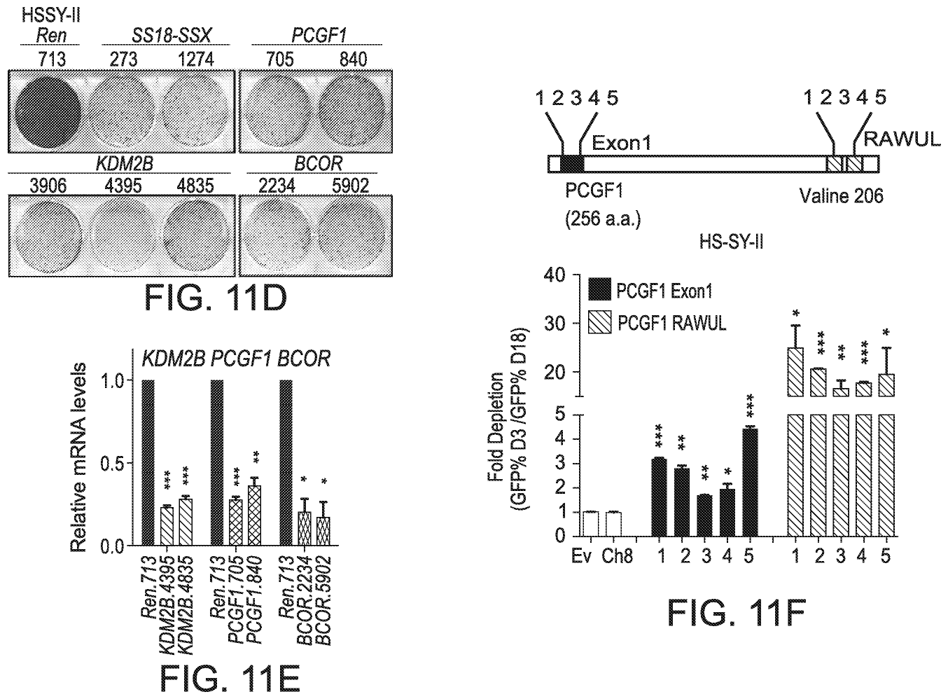

KDM2B encodes a histone demethylase that can repress gene expression by demethylating H3K36me2 (He et al., 2008; Tzatsos et al., 2009). In addition, KDM2B is a core component of a poorly understood non-canonical polycomb repressive complex (BCOR complex or PRC1.1)(Gearhart et al, 2006) that, unlike the canonical PRC1, can be recruited to polycomb target sites in a PRC2-independent manner (Blackledge et al., 2014). Whereas the KDM2B demethylase activity requires a JmjC domain, its role in recruiting PRC1.1 involves binding to unmethylated CpG islands (CGIs) via its zinc finger-CxxC (ZF-CxxC) domain (Farcas et al., 2012; He et al., 2013; Wu et al., 2013). To determine whether either or both of these domains are required for synovial sarcoma maintenance, a structure-function analysis was performed that exploits a nuance of CRISPR-Cas9-directed mutagenesis whereby guide RNAs (sgRNAs) targeting essential domains show greater depletion than those targeting dispensable regions in competition assays (Shi et al., 2015) (see Methods). To this end, sgRNAs targeting the 5′ exons of KDM2B and regions encoding the JmjC or the ZF-CxxC domains were introduced into five human synovial sarcoma cell lines expressing Cas9 (FIG. 11A).

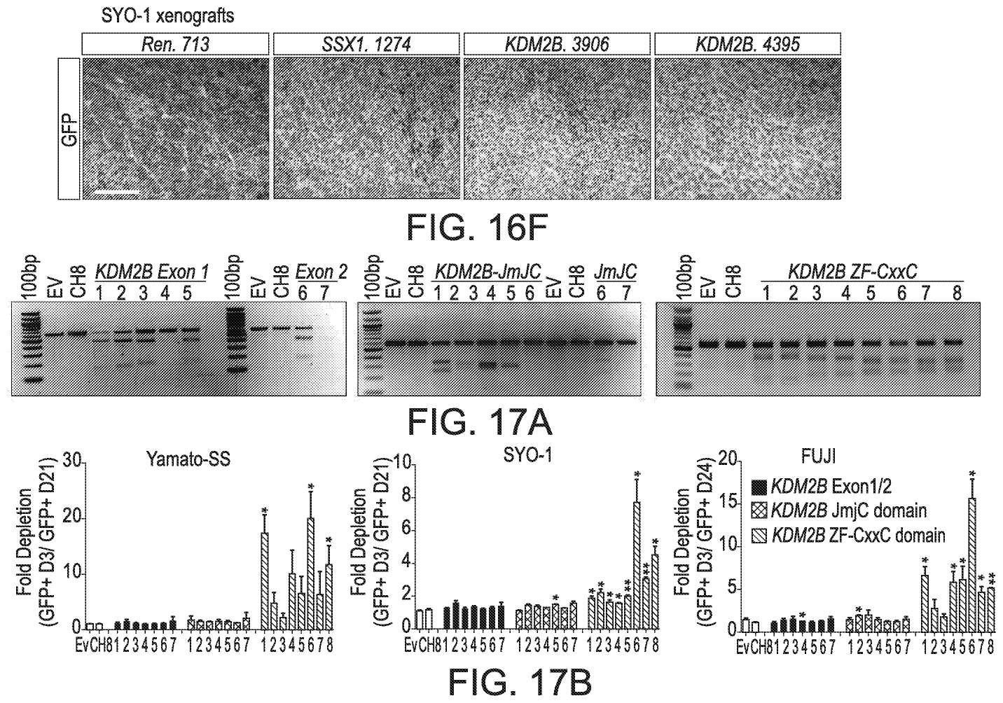

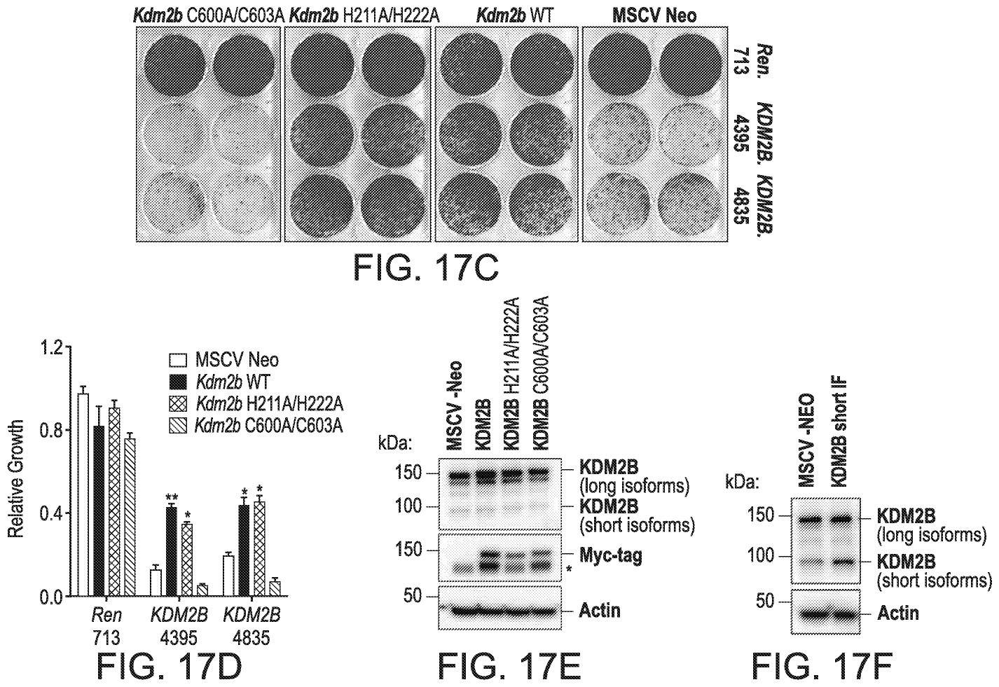

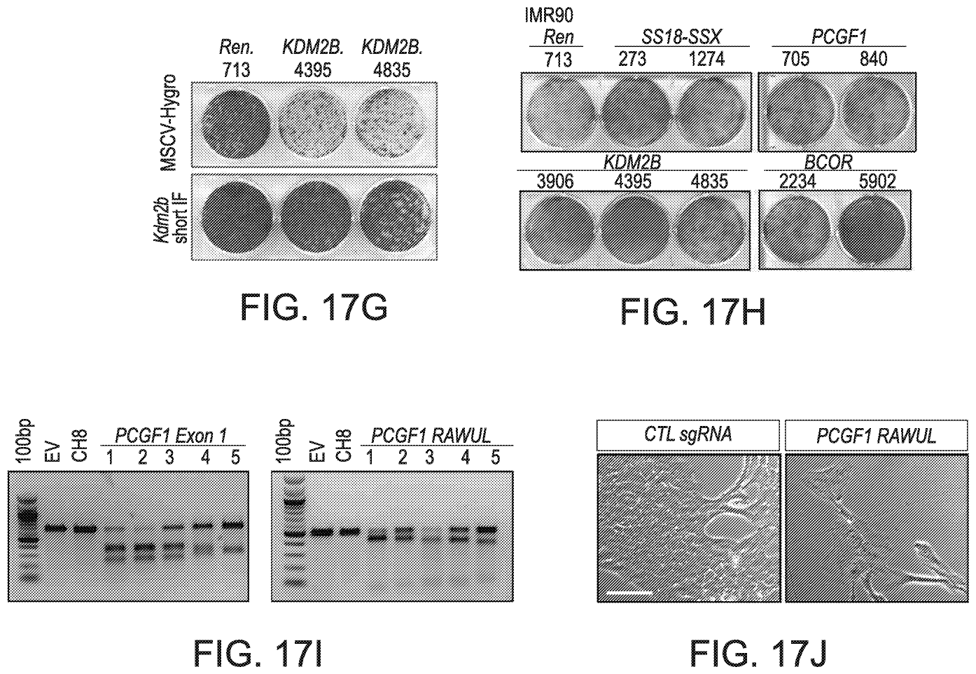

Although most sgRNAs tested were potent at producing mutations (FIG. 17A), sgRNAs targeting the ZF-CxxC motif showed the most significant depletion over time (FIG. 11B and FIG. 17B). These observations imply that the DNA binding domain of KDM2B, and not its histone demethylase activity, is crucial for sustaining synovial sarcoma proliferation and are reminiscent of the prevalent role of KDM2B's ZF-CxxC in promoting gene repression in ESCs cells (He et al., 2013). Accordingly, expression of a JmjC-defective mutant (KDM^{H211A/H222A}) or of a JmjC-deficient short KDM2B isoform was as effective as wild-type KDM2B at rescuing the proliferative arrest produced by KDM2B knockdown (FIGS. 17C-17G).

PCGF1, RING1B, and BCOR (and the related protein BCORL1) are additional components of the PRC1.1 complex that are recruited to unmethylated CGIs by KDM2B (Gao et al., 2012; Sanchez et al., 2007). PCGF1 is specific for PRC1.1 and determines the identity of PRC1-like assembly through its ring finger- and WD40-associated ubiquitin-like (RAWUL) domain (Junco et al., 2013). Intriguingly, like KDM2B, BCOR is also overexpressed in human synovial sarcoma cell lines (FIG. 16A) and patient samples (Kao et al., 2016; Kao et al., 2017), and two Bcor shRNAs also depleted in the initial screen and follow up validation studies (FIG. 15B; note that Pcgf1 or Bcorl1 shRNAs were not present in the library). Furthermore, shRNAs targeting PCGF1 or BCOR induced morphological changes and a proliferative arrest that phenocopied KDM2B knockdown in several human synovial sarcoma cell lines (FIGS. 11C-11E) but did not affect proliferation of normal human fibroblasts (IMR90, FIG. 17H). Reinforcing the importance of the PRC1.1 complex, sgRNAs targeting the RAWUL domain of PCGF1, required for PRC1.1 assembly, strongly depleted in CRISPR-Cas9 competition assays in HS-SY-II cells (FIG. 11F and FIG. 17I-17J). It can be inferred that KDM2B sustains synovial sarcoma through its PRC1.1-associated activity.

Tagging of Endogenous SS18-SSX1 Using CRISPR/Cas9 Homology Directed Repair.

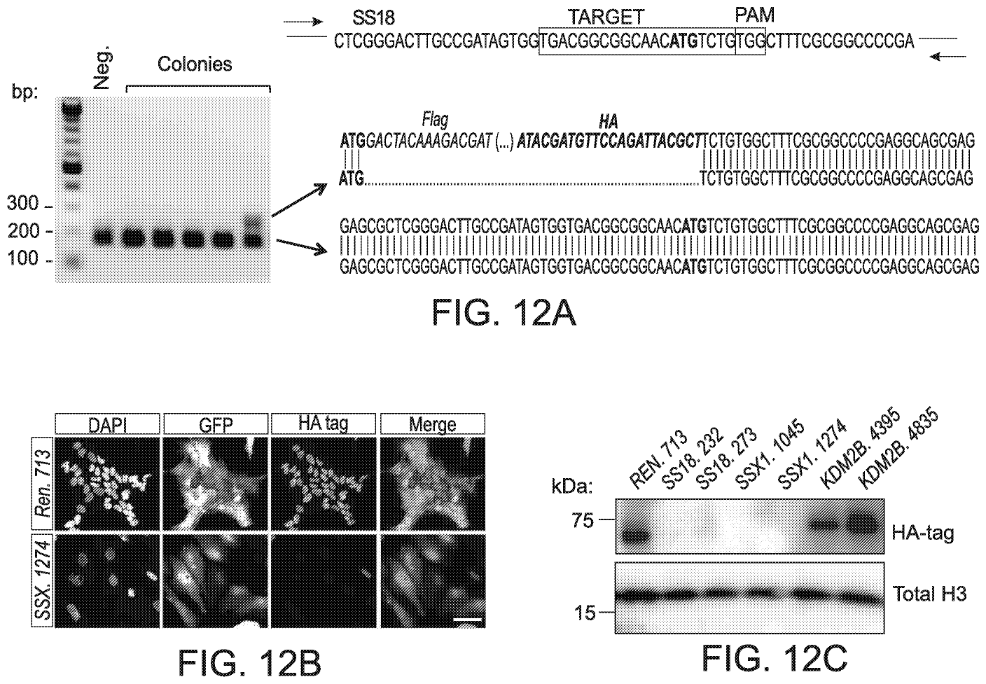

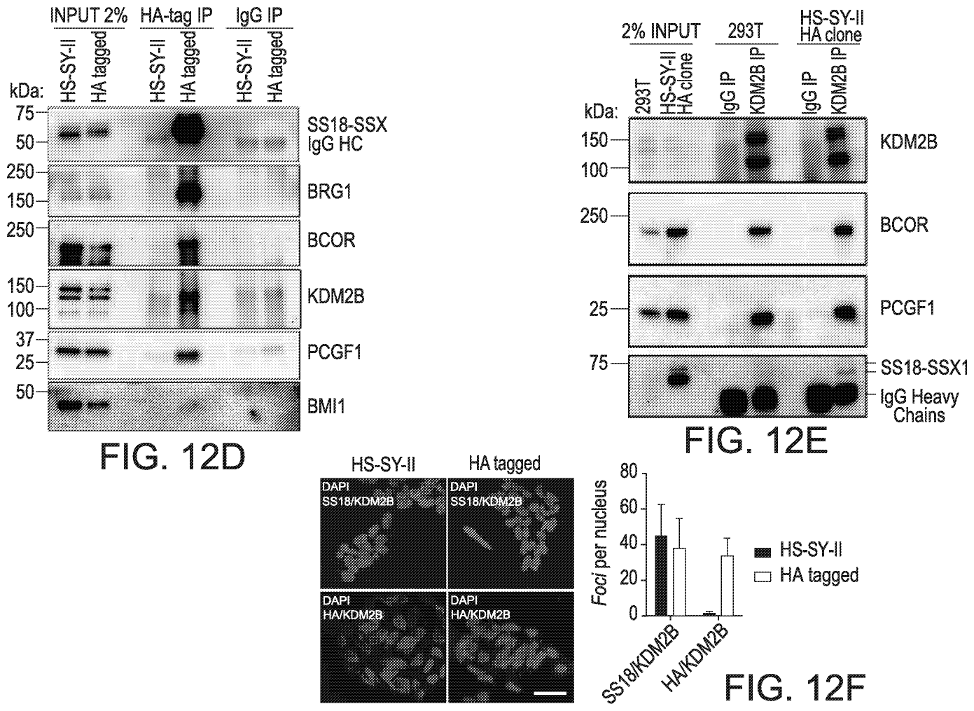

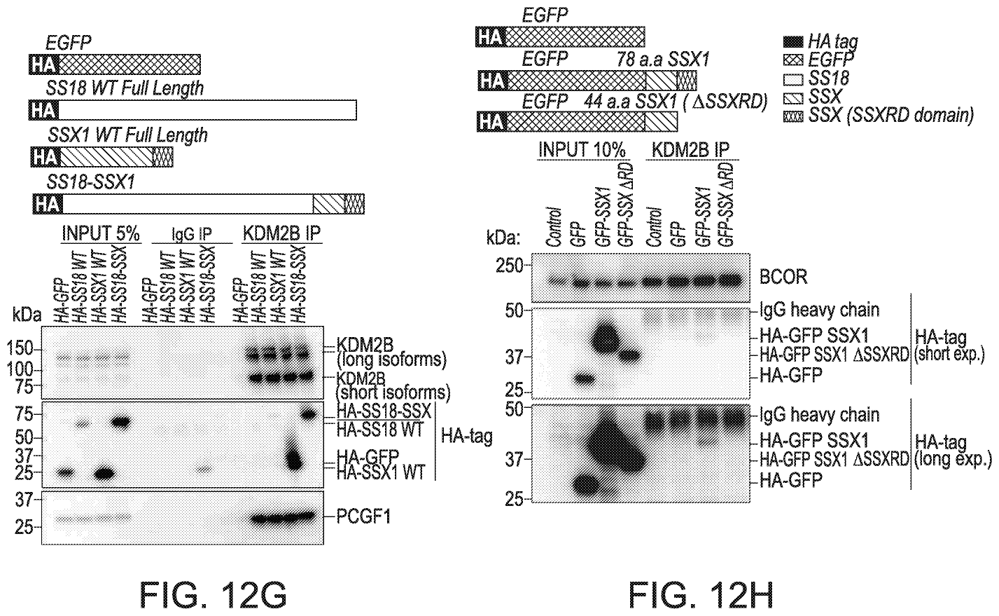

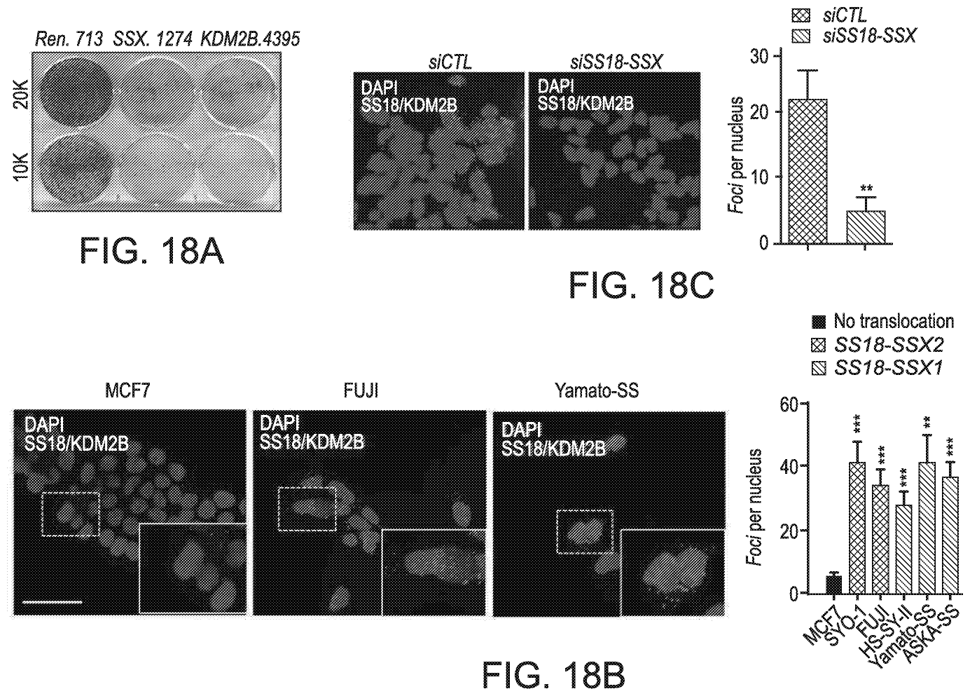

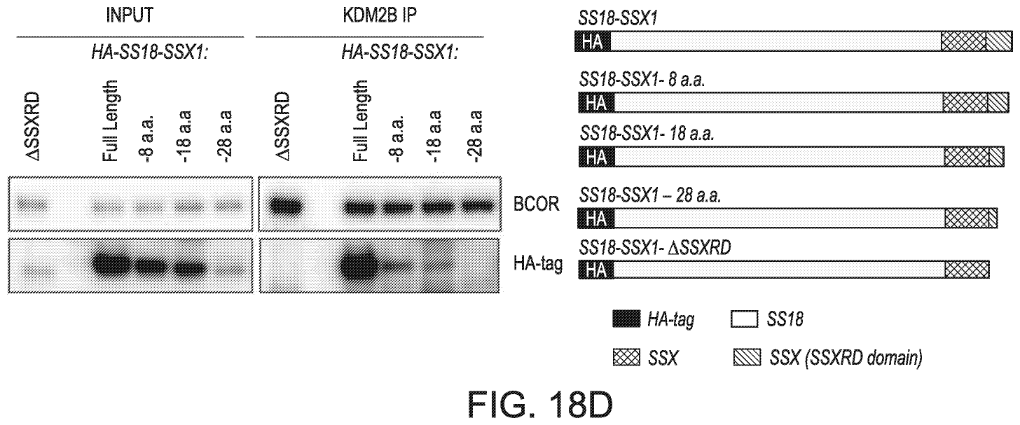

The mechanisms whereby SS18-SSX and KDM2B influence transcriptional regulation in synovial sarcoma cells was investigated. While studies on candidate target genes indicate that SS18-SSX represses the INK4A-ARF (CDKN2A) tumor suppressor locus (Su et al., 2012) and conversely activates SOX2 expression (Kadoch and Crabtree, 2013), the lack of fusion-specific antibodies suitable for chromatin immunoprecipitation (ChIP) has precluded the identification of its targets genome-wide. To overcome this limitation, CRISPR/Cas9 mediated homologous directed repair was applied to knock-in a FLAG-HA tag in the N-terminal region of the SS18 locus in HS-SY-II human synovial sarcoma cells (see Star Methods). A positive clone was identified and confirmed to have edited the SS18-SSXtranslocation without affecting the wild-type SS18 allele (FIG. 12A). Immunofluorescence and immunoblotting confirmed that the HA epitope was depleted by SS18-SSX knockdown (FIGS. 12B and 12C). Importantly, these epitope-tagged cells retained sensitivity to SS18-SSX and KDM2B inhibition (FIG. 18A).

SS18-SSX interacts with KDM2B-PRC1.1 in human synovial sarcoma cells.

It was tested whether SS18-SSX and PRC1.1 components interact by performing reciprocal co-immunoprecipitations (Co-IP) with antibodies targeting HA or KDM2B using buffers that contained DNAse to eliminate physical associations mediated by DNA. Like BRG1, KDM2B, BCOR, and PCGF1 were detected in anti-HA IPs of lysates from the HA tagged but not the parental cell line. No interaction with BMI1 under the same experimental conditions was identified, suggesting the interaction is specific to PRC1.1. (FIG. 12D). SS18-SSX was also identified in IPs using a KDM2B antibody, as were PRC1.1 components (FIG. 12E). The HA-tagged SS18-SSX protein also co-localized with KDM2B in cells as revealed by a proximity ligation assay (PLA) that allows “in situ” detection of two proteins closer than 40 nm (Soderberg et al., 2006) (FIG. 12F). This signal was SS18-SSX-specific and dependent: a strong PLA signal was observed in other synovial sarcoma lines using SS18 and KDM2B antibodies but not in MCF7 cells lacking the fusion (FIG. 18B), and this signal was abolished upon SS18-SSX knockdown using an SSX targeting siRNA (FIG. 18C). KDM2B co-immunoprecipitated with epitope-tagged versions of SSX, but not SS18, in ectopic overexpression assays (FIG. 12G), whereas deletion of the C-terminal SSX repressive domain (SSXRD)(Lim et al., 1998) in the context of the SS18-SSX fusion abolished this interaction (FIG. 18D). Furthermore, addition of the SSX1 fragment of the fusion (last 78 amino acids) to GFP enabled it to associate with KDM2B, in an SSXRD-dependent manner (FIG. 12H). Thus, SS18-SSX directly or indirectly associates with KDM2B via its C-terminal SSX repressive domain.

SS18-SSX1 and KDM2B Co-Occupy Unmethylated CpG islands of Developmental Transcription Factors.

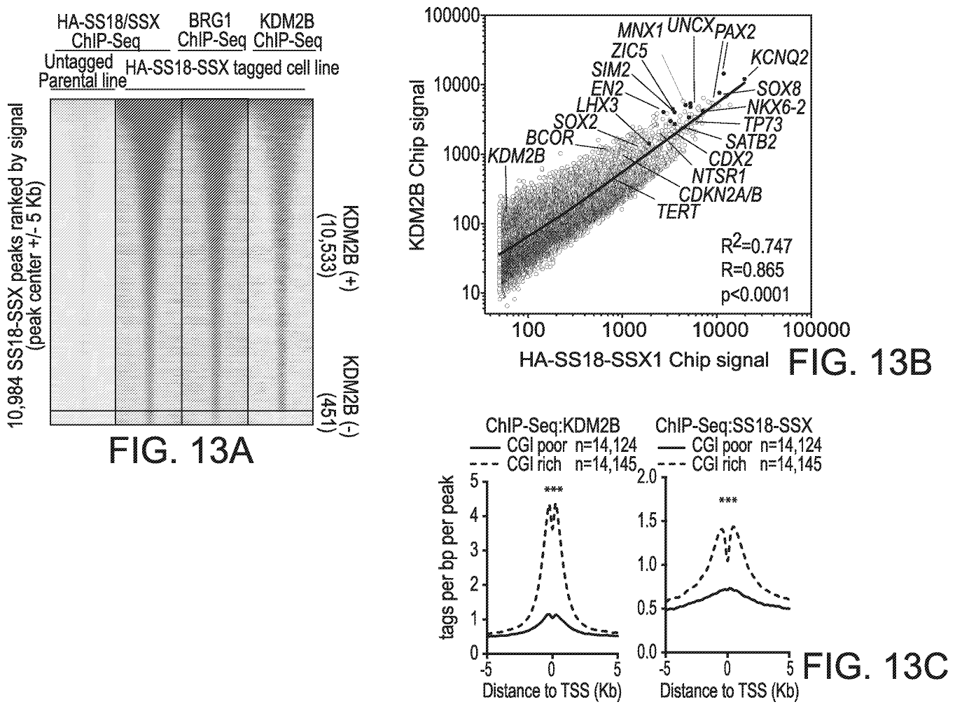

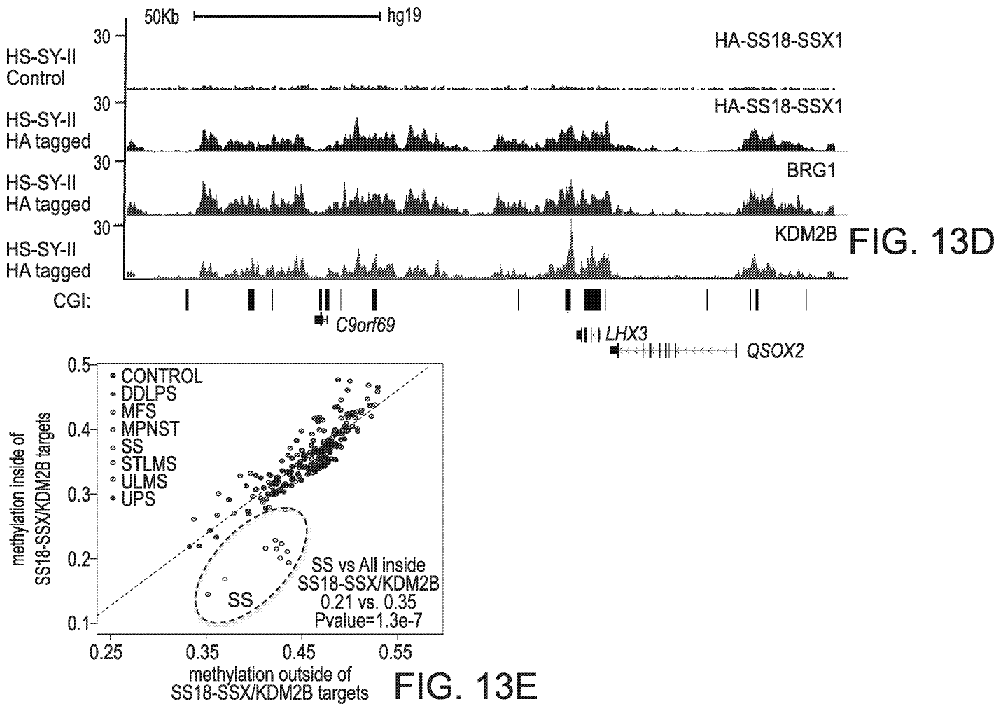

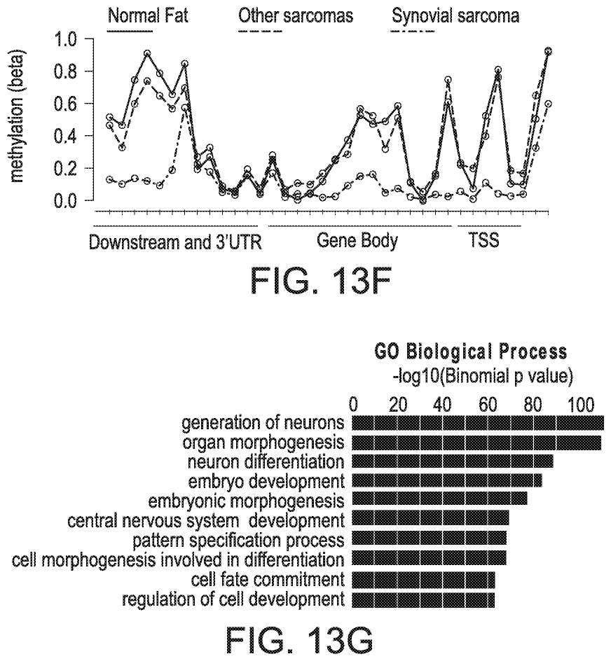

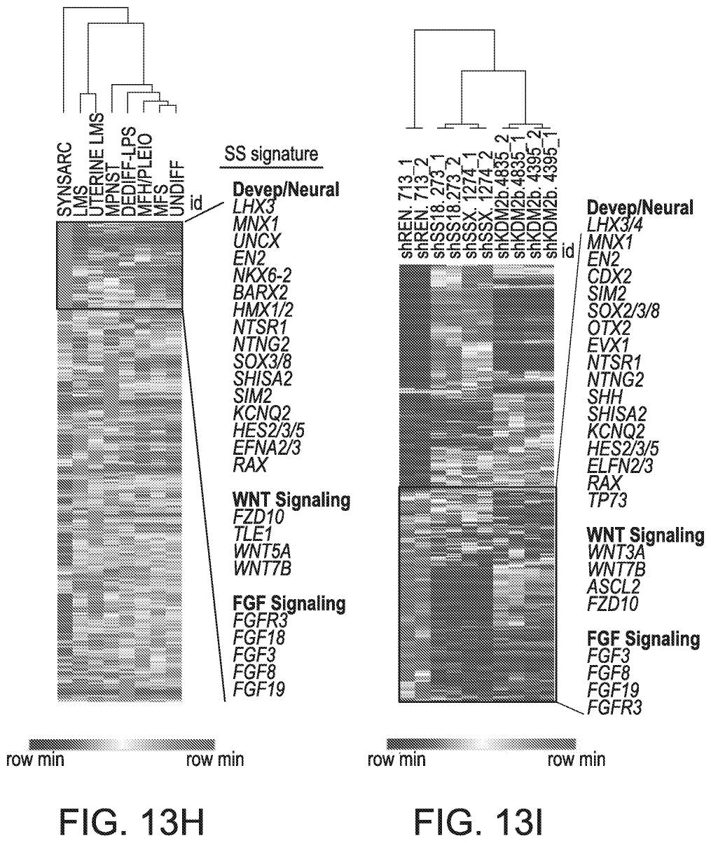

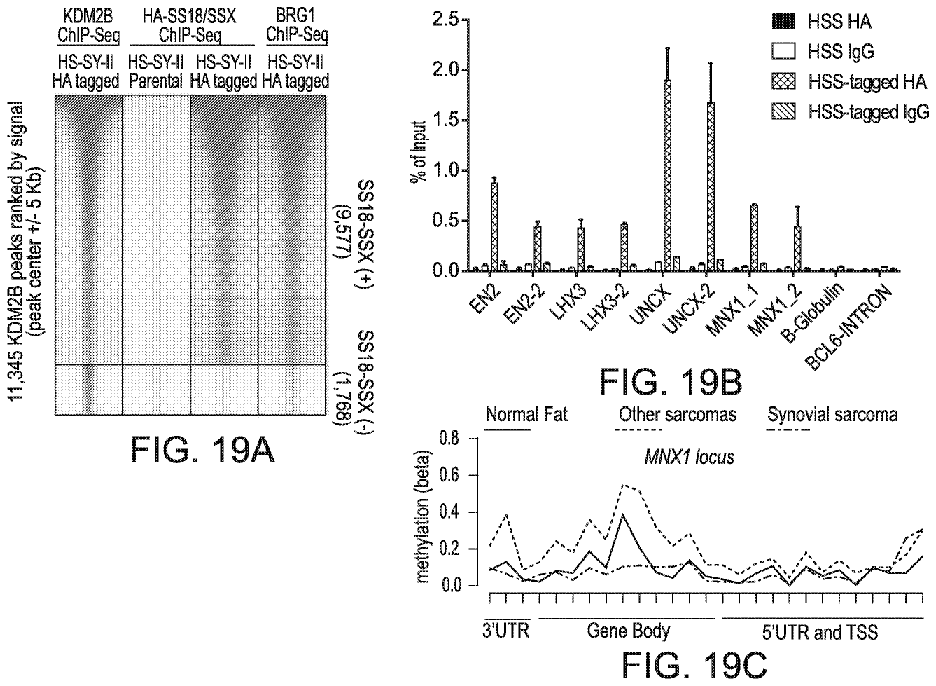

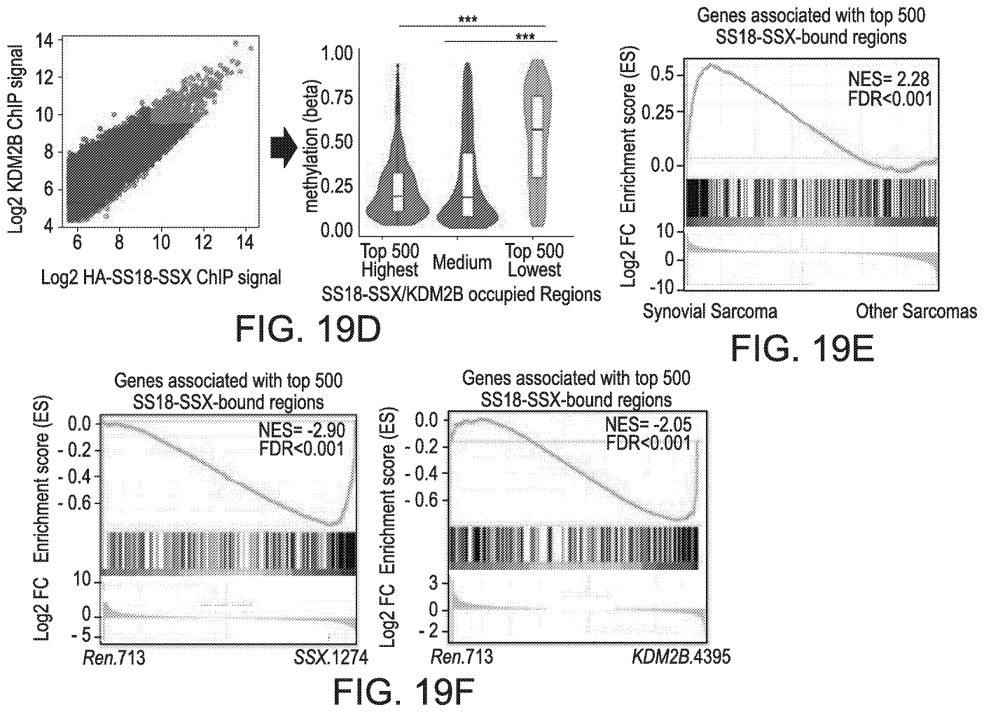

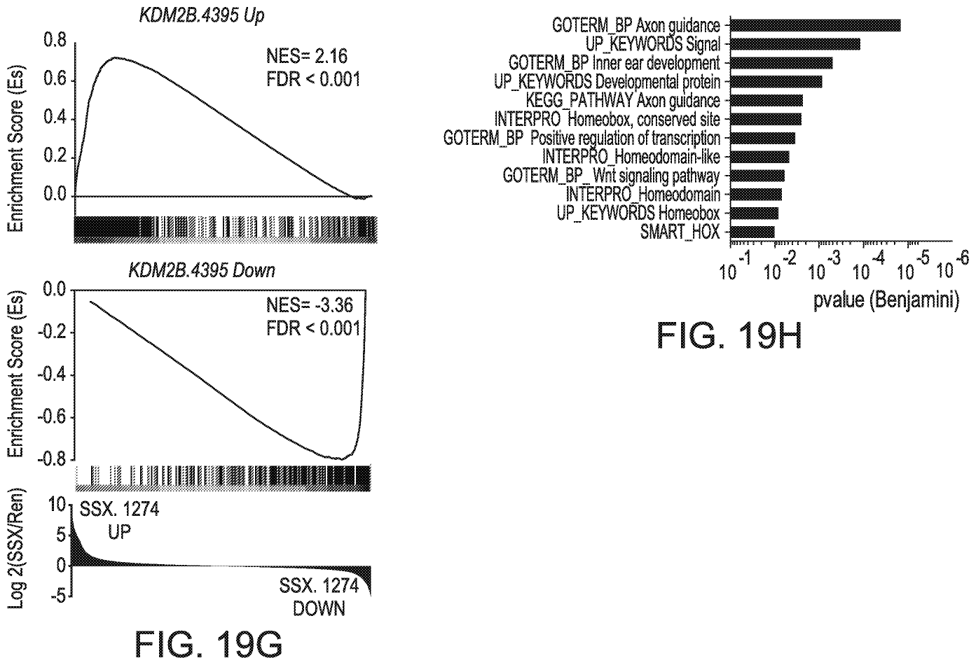

To determine whether SS18-SSX and KDM2B occupy similar loci genome-wide, ChIP-Seq using antibodies targeting HA, BRG1 (a SWI/SNF component), and KDM2B in HA-tagged HS-SY-II cells was performed. 10,984 specific SS18-SSX-occupied regions were identified in HA-tagged cells and, as expected, these loci also contained BRG1 (FIG. 13A). Remarkably, there was a quantitative and significant correlation between loci bound by SS18-SSX and those bound by KDM2B (FIGS. 13A and 13B). Likewise, most KDM2B-bound sites in these cells are also bound by SS18-SSX and BRG1 (84.4% and 97.0% respectively, FIG. 19A).

Consistent with the ability of KDM2B to bind to and recruit proteins to CpG islands, SS18-SSX and KDM2B were bound predominantly to CpG-rich promoters (FIG. 13C) and overlapped with annotated CGIs (FIG. 13D, and FIG. 19B) that are more hypomethylated in synovial sarcoma compared to normal fat or other sarcoma types (FIG. 13E and 13F and FIG. 19C). Furthermore, SS18-SSX/KDM2B occupancy inversely correlated with DNA methylation levels as genes with the highest SS18-SSX/KDM2B enrichment showed the lowest overall methylation levels (FIG. 19D). Therefore, SS18-SSX, SWI/SNF, and KDM2B broadly co-occupy genes linked to unmethylated CGIs, suggesting that KDM2B-mediated recognition of these regions is required for SS18-SSX activity.

High SS18-SSX/KDM2B Occupancy is Associated with Gene Activation.

Although the previously identified SS18-SSX targets CDKN2A/B (Su et al., 2012) and SOX2 (Kadoch and Crabtree, 2013) displayed SS18-SSX and KDM2B occupancy in synovial sarcoma cells, they were not among the most prominently bound loci identified in this analysis (FIG. 13B). Rather, SS18-SSX and KDM2B co-occupancy was higher at genes encoding a series of homeobox transcription factors linked to neurogenesis and other developmental processes (EN2, LHX3, UCNX, PAX2, MNX1, ZIC5, KCNQ2, and SIM2) (FIG. 13B, FIG. 19B) (Hegarty et al., 2013; Lee and Pfaff, 2001) and, accordingly, systematic gene ontology analysis using all co-occupied loci identified “neuron differentiation”, “embryo development” and “homeobox” among the most significant categories (FIG. 13G). Key genes present in these categories were also present in the set of differentially expressed genes that distinguished synovial sarcoma from other sarcoma types (FIG. 13H, FIG. 19E), and the fact that many of these factors are linked to nervous system development likely explains the observation that neurogenesis-related genes are paradoxically upregulated in this disease (Baird et al., 2005; Nagayama et al., 2002). Interestingly, both BCOR and KDM2B are also SS18-SSX/KDM2B targets, suggesting an auto-regulatory mechanism that could produce the high levels of PRC1.1 components observed in synovial sarcoma cells (FIG. 17B).

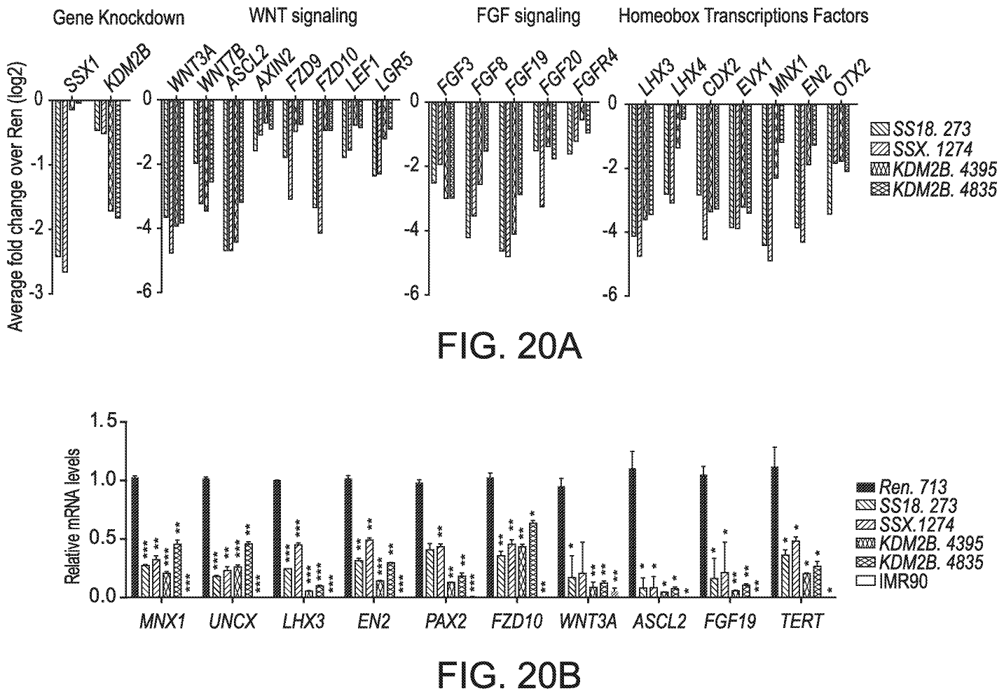

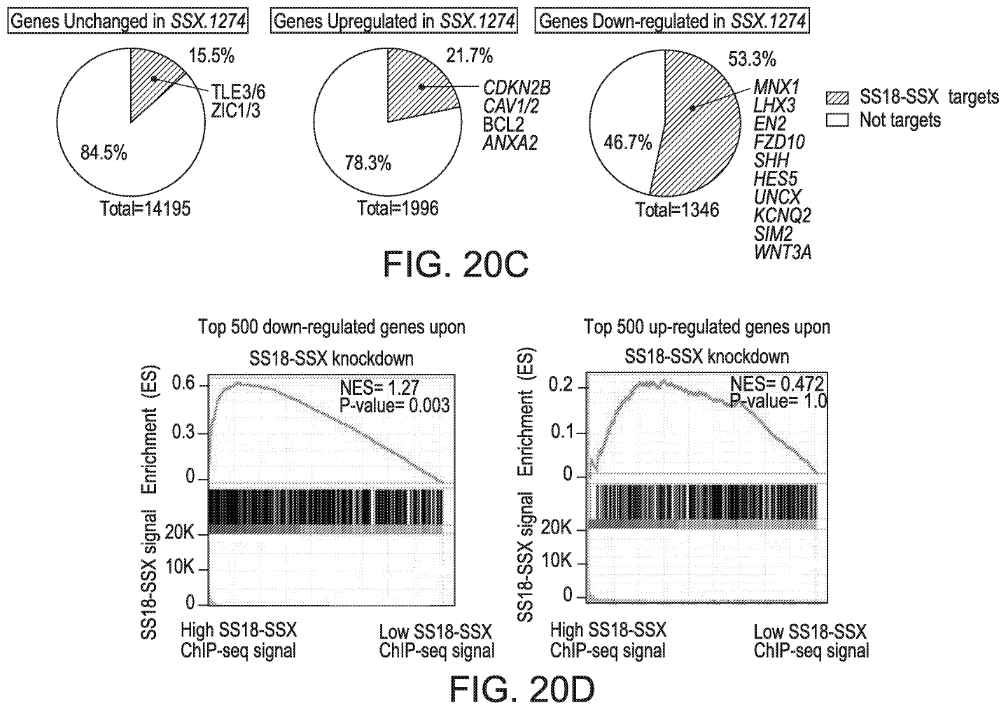

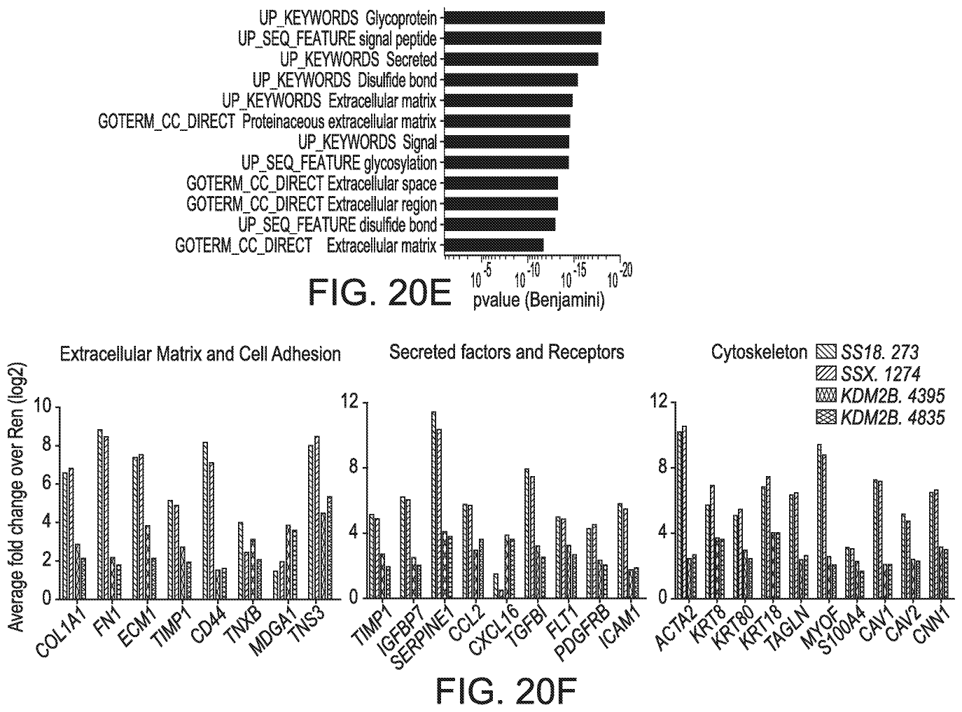

Transcriptional profiling of synovial sarcoma cells transduced with either SS18-SSX or KDM2B shRNAs revealed a strong correlation between genes that were highly bound by both SS18-SSX and KDM2B and whose expression was significantly repressed upon SS18-SSX knockdown (FIG. 19F). Although the magnitude of the expression changes was lower for KDM2B depletion, the impact of each perturbation on gene expression was remarkably similar (FIG. 19G). Co-downregulated genes included the same developmental and neural factors described above, as well as genes involved in FGF and WNT signaling that have been implicated in synovial sarcoma (Barham et al., 2013; Ishibe et al., 2005; Trautmann et al., 2014) (FIG. 13I and FIG. 19H and FIG. 20A-20B). In contrast, most co-upregulated genes were not direct SS18-SSX targets (FIG. 20C) and included genes with lower SS18-SSX/KDM2B-bound levels when compared to down-regulated genes (FIG. 20D). Consistent with the results described in FIGS. 9F and 9G, up-regulated genes included those indicative of mesenchymal differentiation, including genes encoding certain extracellular matrix proteins, secreted factors and cytoskeleton-related proteins (FIGS. 20E and 20F). Apparently, the SS18-SSX/KDM2B collaboration produces the neurogenesis gene expression signature that is a hallmark of synovial sarcoma (Nagayama et al., 2002), and its disruption restores a more mesenchymal cell fate.

SS18-SSX1 Recruitment to Targets Genes Depends on KDM2B

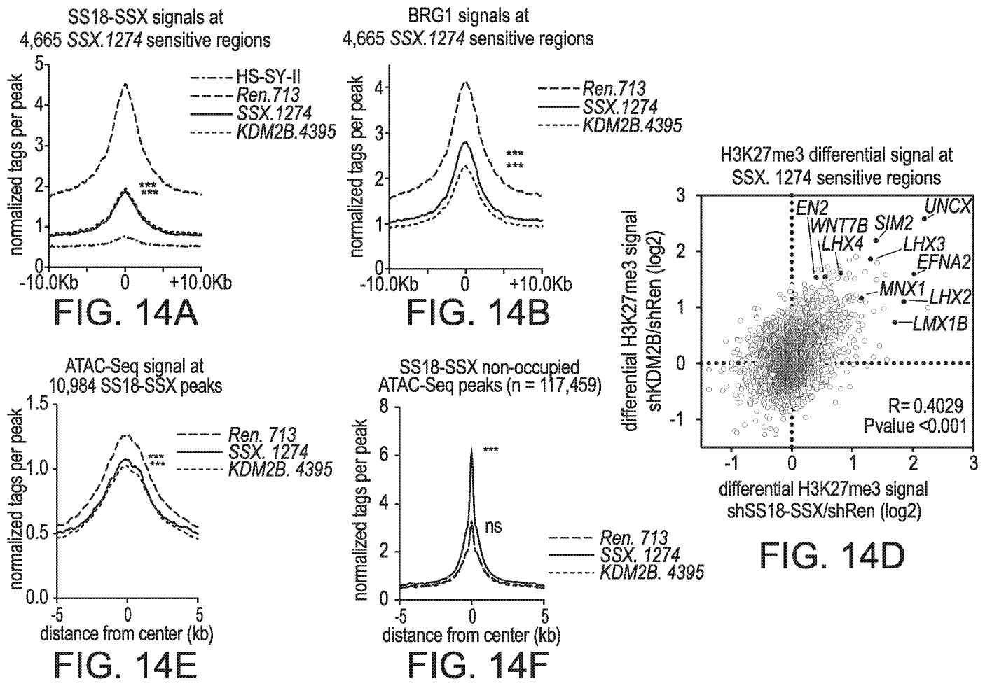

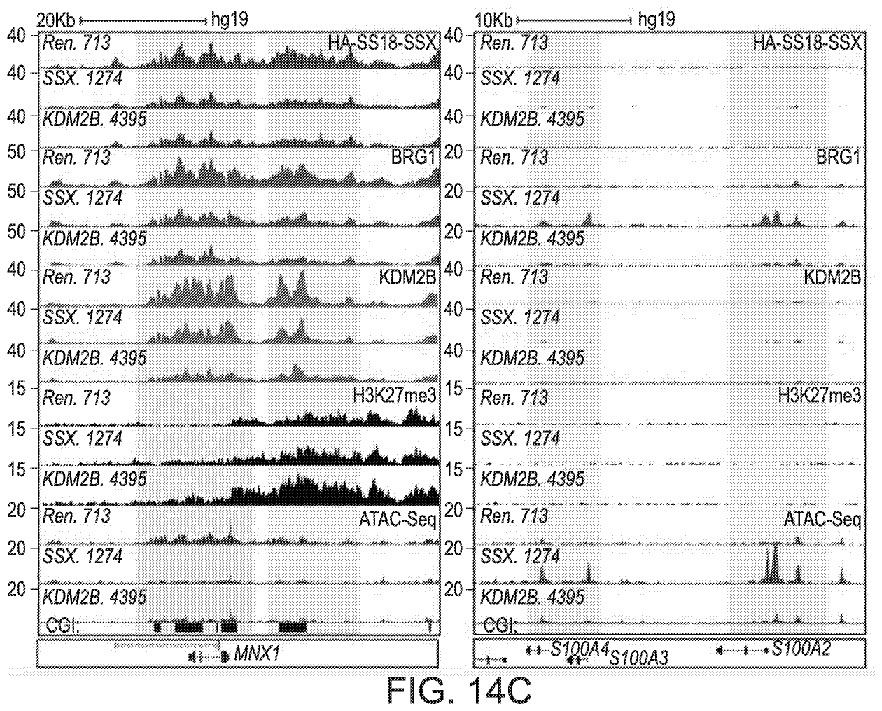

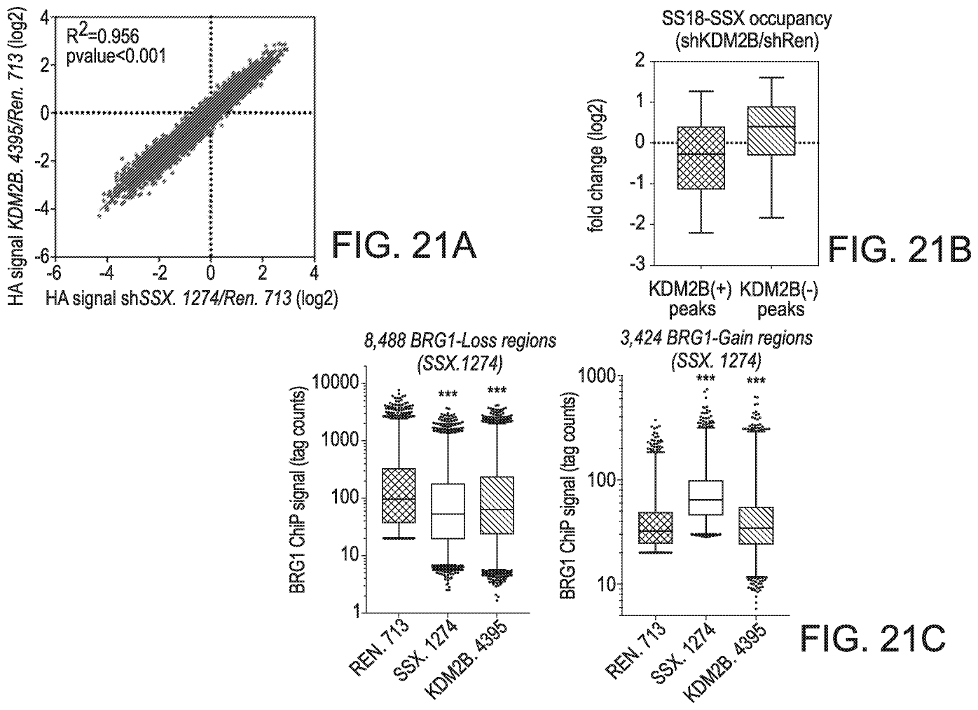

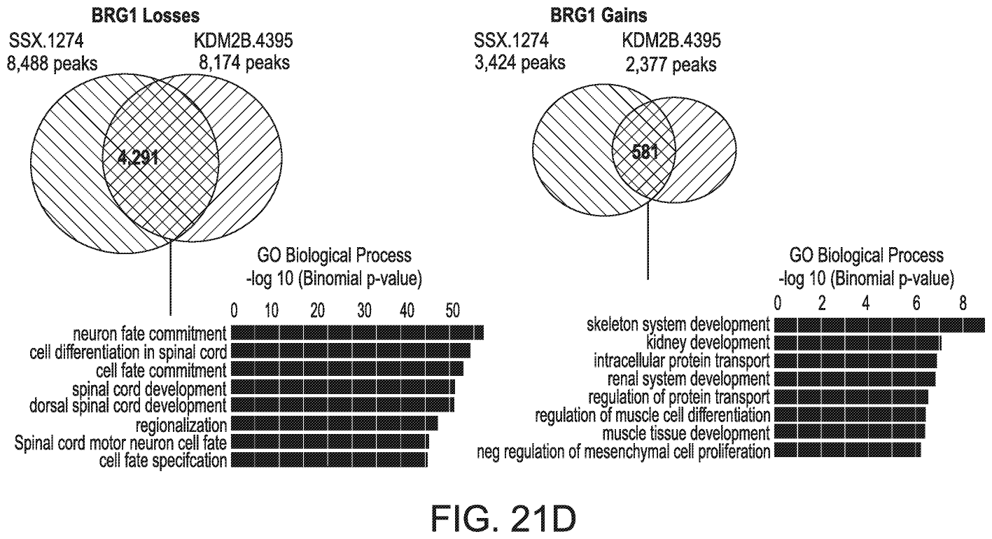

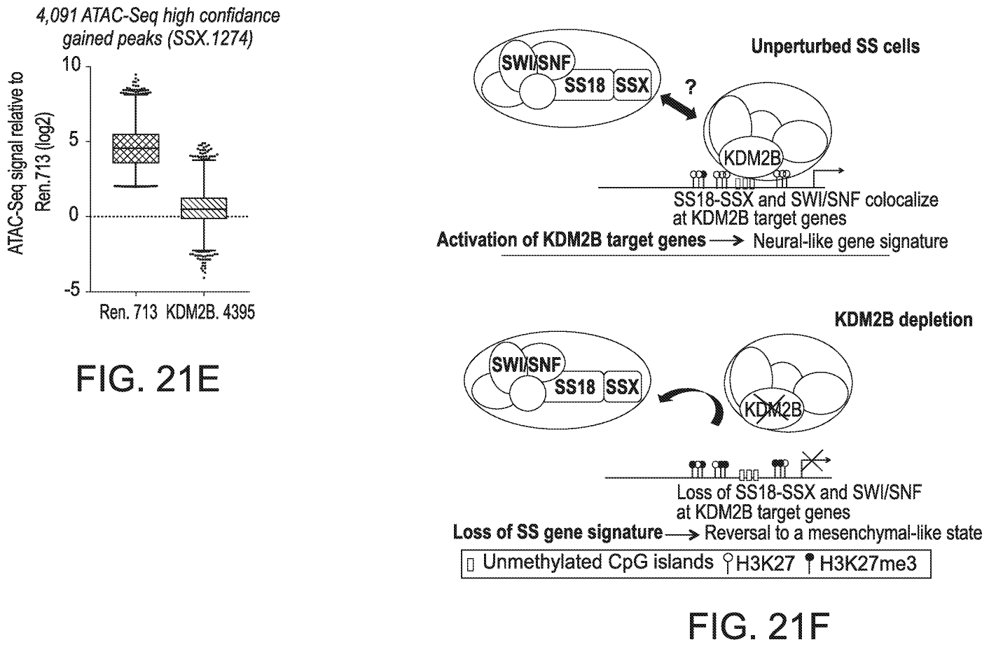

These results raise the possibility that KDM2B-PRC1.1, via binding to CGIs, mediates SS18-SSX recruitment to chromatin thereby dictating target gene identity. To test this hypothesis, we profiled SS18-SSX and BRG1 occupancy following either SS18-SSX or KDM2B knockdown. As expected, SS18-SSX knockdown reduced the HA-SS18-SSX and BRG1 (i.e. SWI/SNF) signal at a large fraction of SS18-SSX target loci (FIGS. 14A-C and FIG. 21A). More importantly, KDM2B depletion also reduced SS18-SSX and BRG1 binding to loci co-occupied by SS18-SSX/KDM2B but not loci bound by SS18-SSX alone (FIG. 14A-C and FIG. 21C-21D). Conversely, knockdown of either SS18-SSX or KDM2B triggered accumulation of the H3K27me3 repressive mark at genomic loci otherwise co-occupied by both proteins, including the same developmentally regulated transcription factors that were targets of the fusion oncoprotein (FIG. 14D).

That SS18-SSX-containing SWI/SNF complexes localize to KDM2B-bound developmental genes expressed in synovial sarcoma cells implies that they promote their gene activation by increasing chromatin accessibility. To address this question directly, Assay for Transposase-Accessible Chromatin sequencing (ATAC-Seq)(Buenrostro et al., 2013) was performed in synovial sarcoma cells in the presence and absence of SS18-SSX or KDM2B. As predicted both SS18-SSX and KDM2B knockdown resulted in reduced ATAC-Seq signals at regions that were previously occupied by SS18-SSX (FIG. 14E). These data support a model in which recruitment of SS18-SSX-containing SWI/SNF complexes via KDM2B promotes gene accessibility by opposing H3K27me3-mediated repression.

SS18-SSX and, to a lesser extent, KDM2B knockdown also produced increases in BRG1 signals at a series of new loci associated with genes involved in skeleton and muscle development, including several of the mesenchymal genes we previously noted as upregulated by SS18-SSX or KDM2B knockdown (e.g. S100A4; FIG. 21C-21D). These regions overlapped with regions that gained gene accessibility (FIG. 14C) and, accordingly, ATAC-Seq signals were generally increased at non-SS18-SSX occupied regions, particularly upon SS18-SSX depletion (FIG. 14F and FIG. 21E). Hence, both SS18-SSX and KDM2B inhibition in synovial sarcoma cells triggered a broad reduction of chromatin accessibility at SS18-SSX/KDM2B targets and a redistribution of SWI-SNF complexes to new loci. Collectively, these data imply that SS18-SSX sustains synovial sarcoma by targeting SWI-SNF complexes to polycomb repressive sites via KDM2B. Consequently, KDM2B inhibition triggers cell cycle arrest and terminal differentiation by releasing SS18-SSX gene activation complexes and allowing the formation of a repressive chromatin environment at target loci.

Discussion

Through functional genomics and mechanistic studies, we identify KDM2B as an epigenetic dependency in synovial sarcoma and reveal how it mediates the oncogenic activity of SS18-SSX. It is proposed that the KDM2B-containing PRC1.1 complex promotes recruitment of SS18-SSX-containing SWI/SNF complexes to unmethylated CpG islands normally subject to polycomb-mediated repression. This process, in turn, enhances gene accessibility leading to aberrant activation of developmentally regulated genes that drive malignancy and underlies the unique transcriptional landscape observed in synovial sarcoma. KDM2B inhibition reverses this program by releasing SS18-SSX from chromatin, thereby enabling target gene silencing, re-acquisition of a mesenchymal expression programs, and irreversible proliferative arrest (FIG. 21F). While the biochemical details of how SS18-SSX associates with PRC1.1 remains to determined, it depends on the SSX fragment and its C-terminal SSXRD domain.

Previous work suggests that SS18-SSX can repress gene expression by enhancing PRC2 activity (Su et al., 2012) or, alternatively activate gene expression at the Sox2 locus by virtue of evicting PRC complexes and repressive marks (Kadoch and Crabtree, 2013). Importantly, these studies focused on single candidate loci and/or relied on ectopic overexpression of SS18-SSX. Furthermore, until now, it has been unclear how SS18-SSX targets SWI/SNF to polycomb-repressed genes. By epitope tagging the endogenous SS18-SSX fusion in human sarcoma cells, we were able to evaluate SS18-SSX occupancy and its impact on gene expression genome wide. It is observed that the most prominent mechanism by which SS18-SSX alters target gene expression is by redirecting SWI/SNF to polycomb targets genome wide via KDM2B. Interestingly, KDM2B promotes gene silencing of developmental genes in embryonic stem cells and in some tumorigenic contexts (Andricovich et al., 2016; Farcas et al., 2012; He et al., 2013; Wu et al., 2013). However, in synovial sarcoma, SS18-SSX connects SWI/SNF to PRC1.1, turning a non-canonical repressive complex into a potent activator that sustains transformation.

In other cell types, SWI/SNF can oppose polycomb repression by binding and evicting of RYBP-containing PRC1 complexes from chromatin (Stanton et al., 2017). We show that, in synovial sarcoma cells, SS18-SSX achieves the same end via an entirely different mechanism—by targeting KDM2B PRC1.1 complexes, the SS18-SSX fusion facilitates mislocalization of SWI/SNF complexes to polycomb target genes where, as previously described (Kadoch et al., 2017; Stanton et al., 2017), it opposes polycomb repressive activity This mechanism leads to the aberrant activation of multiple neurogenic and other transcription factors, which likely explains the curious transcriptional profile associated with synovial sarcoma (Baird et al., 2005; Nagayama et al., 2002).

These results do not rule out the possibility that altered SWI/SNF composition or gene repression mechanisms contribute to the oncogenic activity of SS18-SSX. Biochemical studies strongly suggest that SS18-SSX containing SWI/SNF complexes have potentiated activity (Kadoch et al., 2017) and it seems likely that this can potentiate the activity of SS18-SSX containing SWI/SNF complexes that are recruited to PRC1.1 targets. In addition, while most repressed genes in SS18-SSX driven sarcoma cells are repressed via indirect mechanisms, our analysis identifies a subset that are direct SS18-SSX targets. Most of those genes were also occupied by BRG1 and KDM2B, though the magnitude of SS18-SSX binding was substantially reduced compared to targets that are directly activated by SS18-SSX. Perhaps other factors such as transcription factor availability, alterations in the balance of PcG to SWI/SNF activity at particular loci, or increased accessibility to transcriptional repressors ultimately dictate the expression output at SS18-SSX target genes. Nevertheless, and although SS18-SSX/KDM2B can be associated with both gene activation and repression, our results show that SS18-SSX's dominant activity is to promote gene activation to produce the synovial sarcoma-gene signature that defines and most probably drives the disease.

By virtue of its ZF-CxxC domain, KDM2B has the ability to specifically recognize non-methylated DNA and to recruit chromatin-modifying activities to CGI elements (Long et al., 2013). Accordingly, in synovial sarcoma, 5518-SSX/KDM2B bind CGI rich genes that are undermethylated in synovial sarcoma patient samples when compared with other sarcoma sub-types. The present model predicts that the impact of SS18-SSX on gene expression will be dictated by the DNA methylation state of the target cell and is intriguing given previous work demonstrating that ectopic SS18-SSX1/2 expression can result in distinct biological outputs and gene expression profiles depending on the cell type (Tamaki et al., 2015). These results may explain why SS18-SSX expression does not faithfully recapitulate its activity in synovial sarcoma cells when expressed in certain cell types, and underscore the importance of studying gene fusions in the pathological context in which they occur. By extension, the combination of functional genomics and CRISPR/Cas9-mediated epitope tagging used herein might be effective at elucidating the mechanism of action of other oncogenic fusions.

Since KDM2B protects CGIs from hypermethylation during embryonic development (Boulard et al., 2015) and is required for SS18-SSX recruitment to hypomethylated CGIs, it is possible that particular methylation states present in the cell of origin create a permissive state for SS18-SSX-driven transformation. Such a model parallels recent findings in Ewing sarcoma, in which DNA methylation patterns in patient samples potentially reflect the differentiation state of the cell-of-origin from which the tumor was originally derived (Sheffield et al., 2017). Consistent with this view, studies directing SS18-SSX transgenes to various cell types in the skeletal muscle lineage demonstrate that only a subset of cell types are sensitive to oncogenic transformation (Haldar et al., 2007). Regardless, the potential role of intrinsic DNA methylation states in SS18-SSX driving transformation may explain why synovial sarcoma occurs at a particular stage of human development.

Beyond KDM2B, our study reveals the broader importance of the PRC1.1 complex in synovial sarcoma. Indeed, like KDM2B, other PRC1.1-specific complex proteins BCOR and PCGF1 co-immunoprecipitate with SS18-SSX, and their disruption phenocopies KDM2B loss on synovial sarcoma cell proliferation and cell fate. Beyond synovial sarcoma, BCOR is translocated to different genes in undifferentiated pediatric sarcomas (BCOR-CCNB3, BCOR-MAML3, ZC3H7B-BCOR)(Peters, 2015; Pierron, 2012; Specht et al., 2016). Additionally, in-frame internal tandem duplications (ITDs) in the PUFD domain of BCOR that interacts with PCGF1 have recently been found in up to 85% of pediatric clear cell sarcoma of the kidney (Roy et al., 2015; Ueno-Yokohata et al., 2015) and in a class of primitive neuroectodermal tumors (CNS-PNET)(Sturm et al., 2016). The repercussions of these mutations on PRC1.1 complex activity are currently unknown, but their presence suggests that alterations in developmental programs controlled by this non-canonical complex broadly contribute to cancer and in particular, pediatric malignancies.

Due to the limited understanding of synovial sarcoma pathogenesis, clinical management consists primarily of surgery and radiotherapy While strategies to target EZH2 activity are promising strategies for the treatment of SWI/SNF-deficient tumors owing to an increased dependence on PRC2 (Kim et al., 2015), our data suggest that, in synovial sarcoma, direct transcriptional repression is a less prominent feature of SS18-SSX action than its ability to promote gene activation via PRC1.1. As a consequence, alternative strategies to target KDM2B or the broader PRC1.1 complex would more broadly disrupt the genome-wide transcriptional impact of the fusion oncoprotein. While these results suggest that enzymatic inhibition of KDM2B's histone demethylase function might not prove efficacious, targeted degradation of KDM2B protein, disruption of KDM2B DNA binding, and targeted inhibition of PRC1.1 complex assembly (e.g. by targeting PCGF1 RAWUL domain (Junco et al., 2013) warrant further investigation. While challenging, the present results imply such approaches would have potent anti-tumor effects by disrupting aberrant differentiation programs and driving synovial sarcoma cells into a non-malignant cell fate.

### Example 3

### Methods

Cell Lines

M5SS1 synovial sarcoma cells used for shRNA screen were derived from a murine synovial sarcoma and provided by K B Jones and M R Capecchi (Haldar et al., 2007). Human synovial sarcoma cell lines: HS-SY-II (Sonobe et al., 1992), YaFUSS (Ishibe et al., 2005), SYO-1 (Kawai et al., 2004), FUJI (Nojima et al., 1990) and Yamato-SS (Naka et al., 2010) cells were provided by M. Ladanyi and T. Nielsen. Cells were authenticated by quantitative PCR detection of SS18-SSX1/2, which is specific to synovial sarcoma (FIG. 16A). Murine myoblasts (C2C12) and human diploid fibroblasts (IMR90, passage 11) were purchased from the American Type Culture Collection (ATCC). Cells were maintained in a humidified incubator at 37° C. with 5% CO₂, grown in DMEM supplemented with 10% FBS and 100 IU/ml penicillin-streptomycin.

Patient Tumor Samples.

Immunohistochemistry was performed on formalin-fixed, paraffin-embedded tissue microarrays (TMAs) from the University of British Columbia: TMA 01-003 (synovial sarcoma and differential diagnoses, 82 cases in duplicate) (Nielsen et al., 2003); TMA 03-008 (chondroid tumors, 121 cases in duplicate) (Ng et al., 2005); TMA 06-007 (myxoid liposarcomas, 69 cases in triplicate) (Cheng et al., 2009); TMA 09-006 (epithelioid sarcoma and differential diagnoses, 53 cases in duplicate)(Pacheco and Nielsen, 2012); TMA 10-009 (8 alveolar soft part sarcomas, 2 alveolar rhabdomyosarcomas, 2 desmoplastic small round cell tumors, in triplicate) (Pacheco and Nielsen, 2012); TMA 12-004 (BCL2-positive tumors, 35 cases in triplicate) (Endo et al., 2014); TMA 12-006 (translocation-associated sarcomas, 10 cases in duplicate) (Endo et al., 2014); TMA 12-010 (5 dedifferentiated liposarcomas and 5 undifferentiated pleomorphic sarcomas, in duplicate)(Endo et al., 2014); TMA 14-006 (4 myxoid liposarcomas, 3 myxofibrosarcomas, 3 chondrosarcomas, 1 synovial sarcoma, 1 malignant peripheral nerve sheath tumor, in duplicate) (Endo et al., 2015); TMA 14-007 (dedifferentiated liposarcomas with well-differentiated areas, both components for 57 cases in duplicate); and TMA MPNST (malignant peripheral nerve sheath tumor and differential diagnoses, 176 cases in duplicate) (Terry et al., 2007). Cores measuring 1.0 mm (TMA 14-007) or 0.6 mm (all other TMAs) in diameter were derived from representative viable tumor tissue, as identified by a bone and soft tissue subspecialty pathologist (TO Nielsen). TMAs were cut to 4-μm-thick sections, mounted to Fisherbrand™ Superfrost™ Plus charged glass slides (Thermo Fisher Scientific Inc, Waltham, Mass.), and incubated for 1 h at 60° C. (see methods details for details on Immunohistochemical staining and analysis).

DNA Methylation and Gene Expression Analysis Of Human Sarcomas.

RNA-Seq data and DNA methylation data from the Illumina Human Methylation 450K platform was downloaded from the UCSC Cancer Genome Browser (Zhu et al., 2009) for 206 samples in the TCGA Sarcoma cohort (“Comprehensive and Integrated Characterization of Adult Soft Tissue Sarcomas”, submitted). These samples spanned multiple major sarcoma types, including dedifferentiated liposarcoma (DDLPS, n=50), uterine leiomyosarcoma (ULMS, n=27), non-uterine leiomyosarcoma (STLMS, n=53), undifferentiated pleomorphic sarcoma (UPS, n=44), myxofibrosarcoma (MFS, n=17), malignant peripheral nerve sheath tumor (MPNST, n=5) and synovial sarcoma (SS, n=10). We used Level 3 data that had been pre-processed using standard TCGA protocols. DNA methylation from the Illumina Human Methylation 450K platform for 15 samples of non-cancerous, subcutaneous adipose tissue (pre gastric-bypass) from 15 patients made publicly available (Benton et al., 2015) through the EBI ArrayExpress database (E-MTAB-3052) was used for comparison purposes.

Animal Studies

All mouse experiments were approved by the Memorial Sloan Kettering Cancer Center (MSKCC) Animal Care and Use Committee. Female 5- to 7-week-old athymic NCR-NU-NU (Harlan Laboratories) mice were used for animal experiments with HS-SY-II and SYO-1 human cell lines. To generate xenografts, HS-SY-II and SYO-1 cells were transduced with LT3GEPIR inducible shRNA vectors and selected with puromycin as described in the method details section. Cells (10×10⁶) were harvested on the day of use and injected in growth-factor-reduced Matrigel/PBS (50% final concentration). Each mouse flank was injected subcutaneously. Following inoculation, mice were fed a doxycycline diet (Harlan Laboratories) and monitored daily. Caliper measurements started when tumors became visible. Tumor volumes were calculated using the following formula: tumor volume=(D×d²)/2, in which D and d refer to the long and short tumor diameter, respectively. For immunohistochemistry analysis of HS-SY-II and SYO-1-derived xenografts, tumors were harvested at the final time point of measure. Tissues were fixed overnight in 4% PFA, embedded in paraffin, and cut into 5 μm sections. Sections were subjected to haematoxylin and eosin staining, and immunohistochemical staining following standard protocols using an anti-GFP antibody (Cell Signaling, 2956, 1:500).

Pooled RNA Interference Screen

An improved version of a previously described custom shRNA library (Zuber et al., 2011b) targeting 400 (six shRNAs per gene) mouse epigenetic regulators was cloned into the LMN (LTR-miR30-PGK-NeoR-IRES-GFP) vector and transduced in triplicates into M5SS1 mouse synovial sarcoma cell line (Haldar et al., 2007) and C2C12 mouse myoblasts (ATCC). T₀ samples (n=3) were collected three days following transduction. Following sixteen days of culture (Tf), about 30 million shRNA-expressing cells per replicate were collected. Genomic DNA from T₀ and Tf samples was isolated and deep-sequencing template libraries were generated by PCR amplification of shRNA guide strands as previously described (Zuber et al., 2011a). Underrepresented shRNAs (<100 normalized reads) at the T₀ were discarded resulting in a total of 2307 shRNAs for further analysis (see Supplementary information—Table 1 for a list of all shRNAs and corresponding reads). Using election criteria that required an shRNA depletion averaging greater than twofold at Tf, at least two shRNAs targeting the same gene and for scoring to be specific to M5SS1 cells, and not in C2C12, 14 shRNAs were identified (hSS18, Kdm2B (Fbxl10), Brd3, Brd7 and Padi4.

Plasmids and Viral Transduction

All vectors were derived from the Murine Stem Cell Virus (MSCV, Clontech) retroviral vector backbone. miRE-based shRNAs were designed and cloned as previously described (Zuber et al., 2011a) into the LT3GEPIR (TRE3G-GFP-miRE-PGK-PuroR-IRES-rtTA3) vector (Fellmann et al., 2013). The mouse Kdm2b cDNA was subcloned from pVXL-Tomato-Kdm2b vector (provided by A. Tzatsos) into MSCV-Neo. The JmjC and ZF-CxxC were generated from the wild-type Kdm2b vector by site directed mutagenesis (Q5 site-directed mutagenesis kit, New England Biolabs). The short isoform of Kdm2b was amplified by PRC from M5SS1 cDNA and cloned into MSCV-hygro. All constructs were verified by sequencing. For CRISPR editing constructs see CRISPR/Cas9 genome editing section. Lentiviruses were produced by co-transfection of 293T cells with 10 ug LT3GEPIR construct and helper vectors (6.5 ug psPAX2 and 2.5 ug VSV-G). For retroviral infection 293T-gag-pol cells were transduced with 20 ug of MSCV vectors and 2.5 ug of VSV-G. Transfection of packaging cells was performed using Polyethylenimine (PEI) (Polysciences, 23966-2) by mixing with DNA in a 3:1 ratio. Viral supernatants were collected 48 after transfection, filtered through a 0.45 um filter (Millipore) and supplemented with 4 ug/ml of polybrene (Sigma) before adding to target cells.

Competitive Proliferation Assays and Clonogenic Assays

For shRNA experiments, human or mouse cells were modified by retroviral or lentiviral transduction followed by drug selection (2 μg/ml Puromycin or 100 ug/ml Hygromycin B). LT3GEPIR-Puro-shRNA transduced cells were treated with 1 μg/ml doxycycline to induce shRNA expression. For competitive proliferation assays, shRNA-transduced cells were mixed with non-transduced cells (in about a 8:2 ratio) and cultured with doxycycline. The relative percentage of GFP⁺ cells was determined at day 2 after doxycycline (T₀) and after 15-18 days in culture (Tf) (results are relative to T₀). The quantification of fluorescent cells was monitored on a Guava EasyCyte (Millipore). Experiments were performed independently two times. For clonogenic assays, cells were seeded in duplicate (10×10³ to 30×10³ cells per well of a 6-well plate) and cultured in the presence of doxycycline in complete media for 10-14 days. Cells were fixed with glutaraldehyde (0.5%), stained with 0.5% crystal violet, and photographed using a digital scanner. All experiments were performed at least three times. Representative experiments are shown.

sgRNA Design and CRISPR/Cas9 Competition Assays

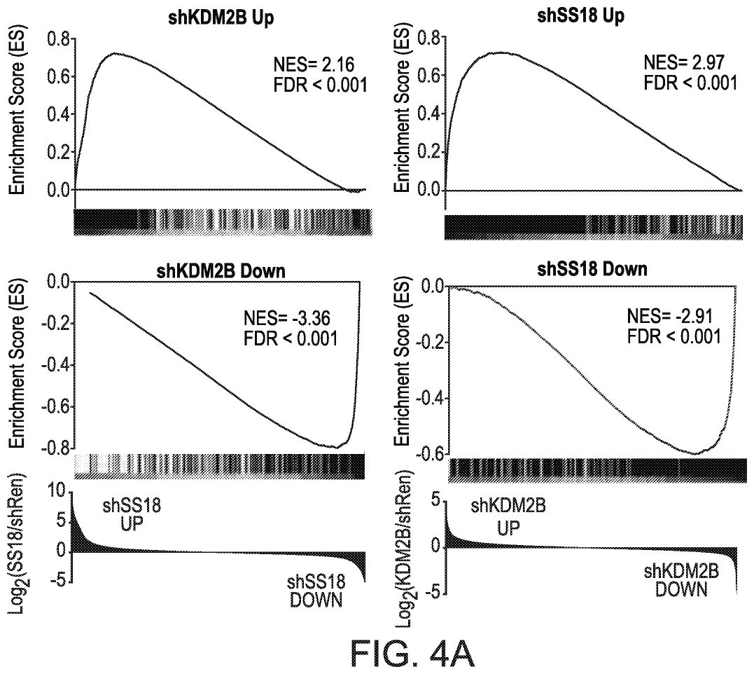

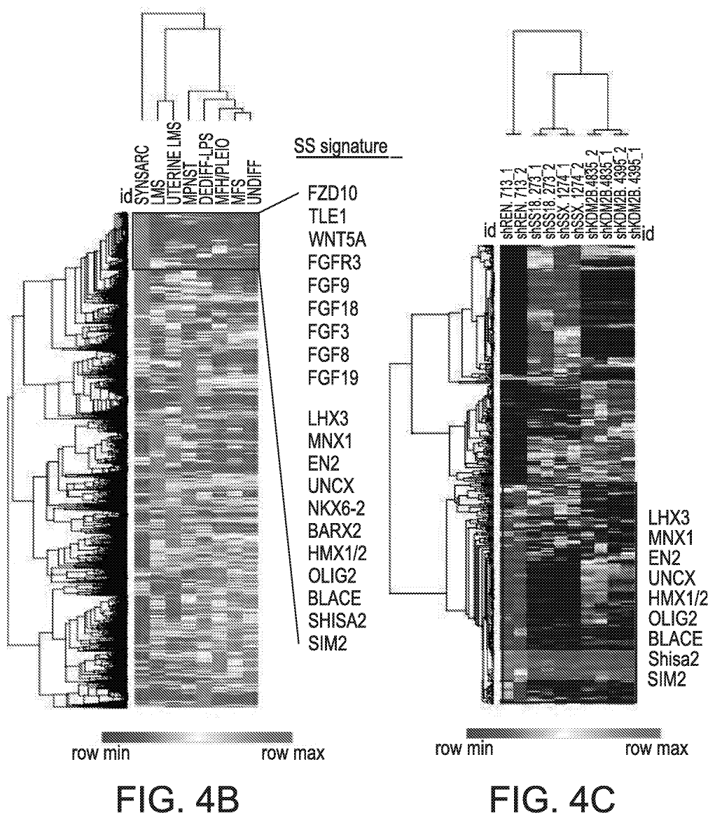

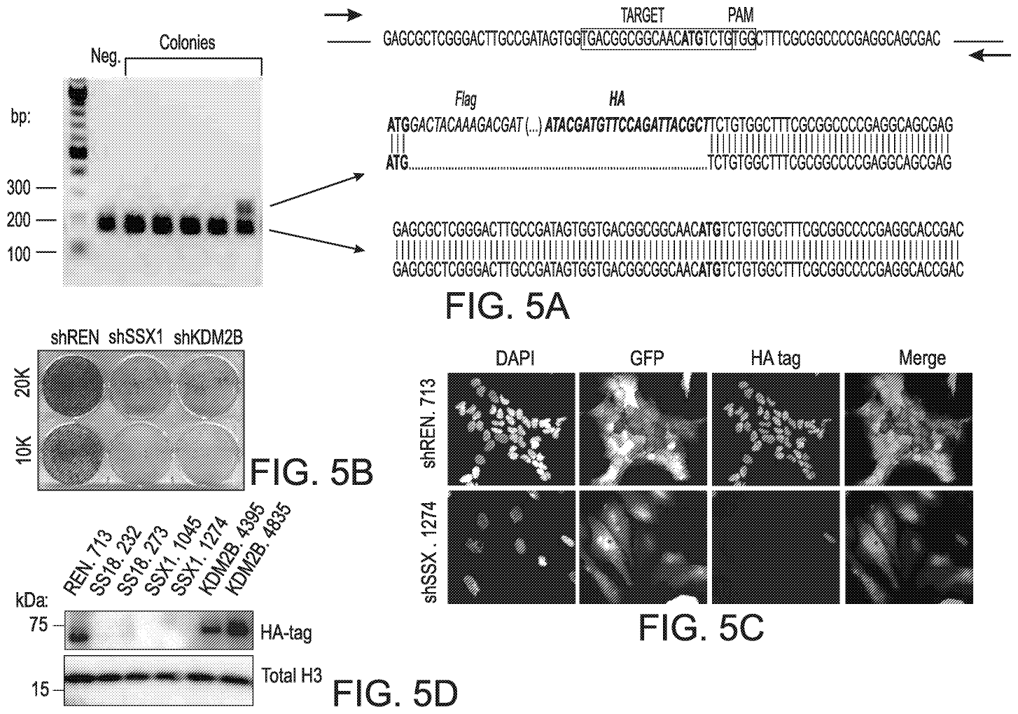

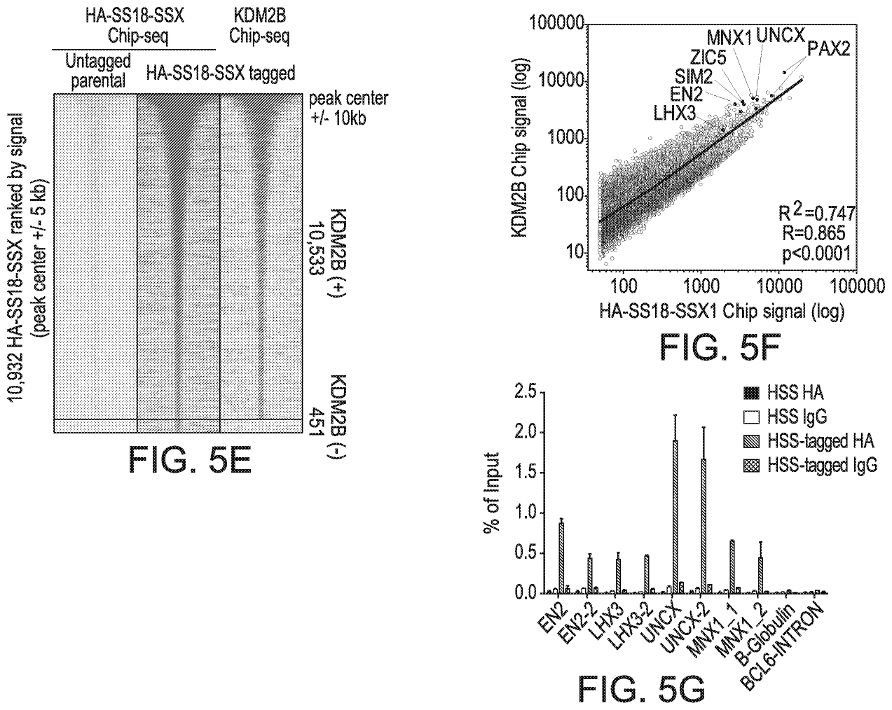

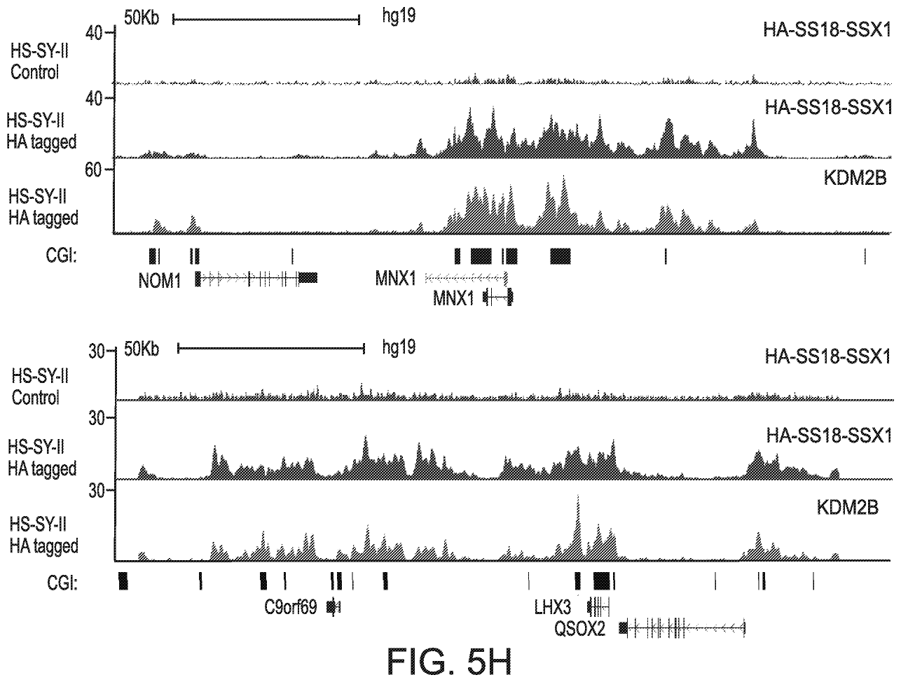

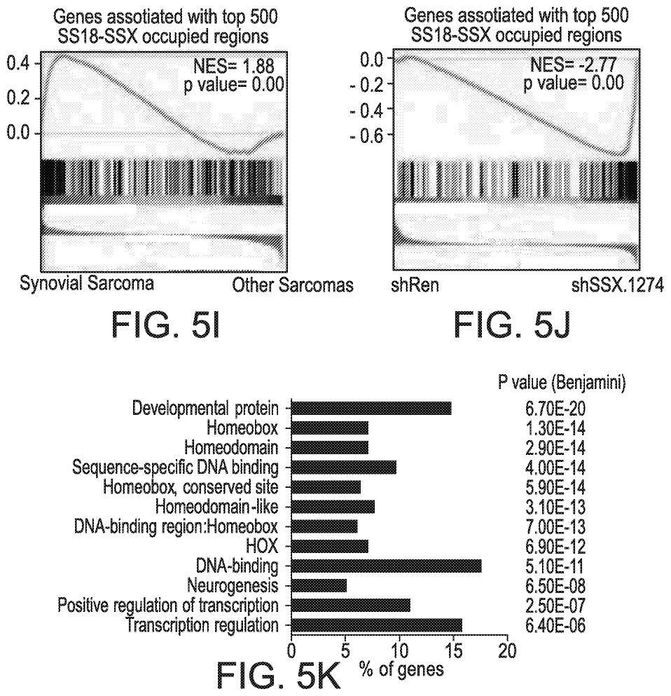

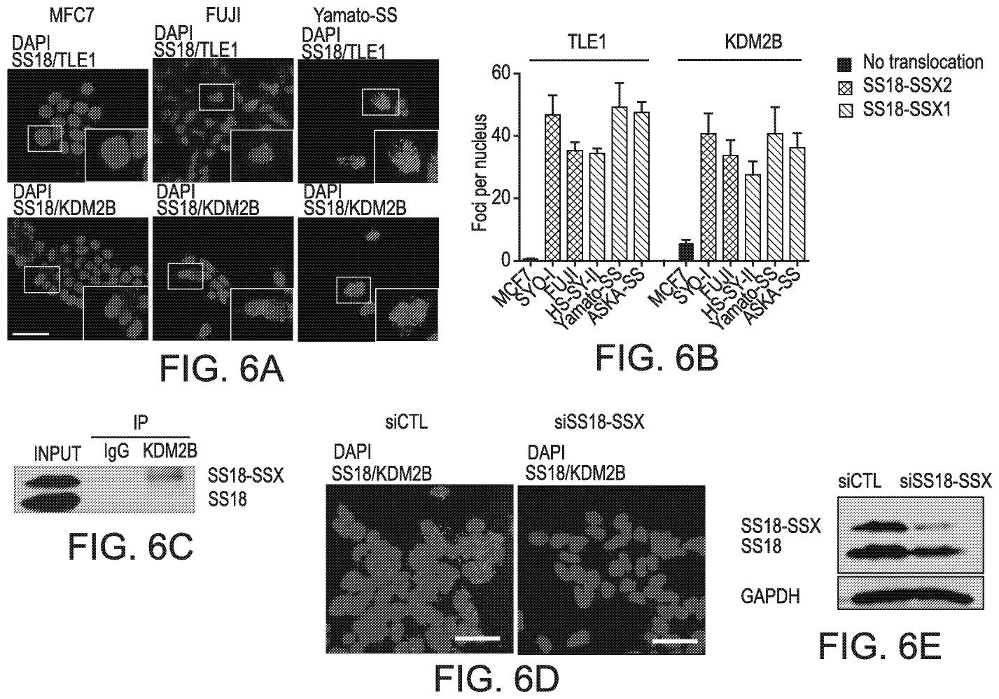

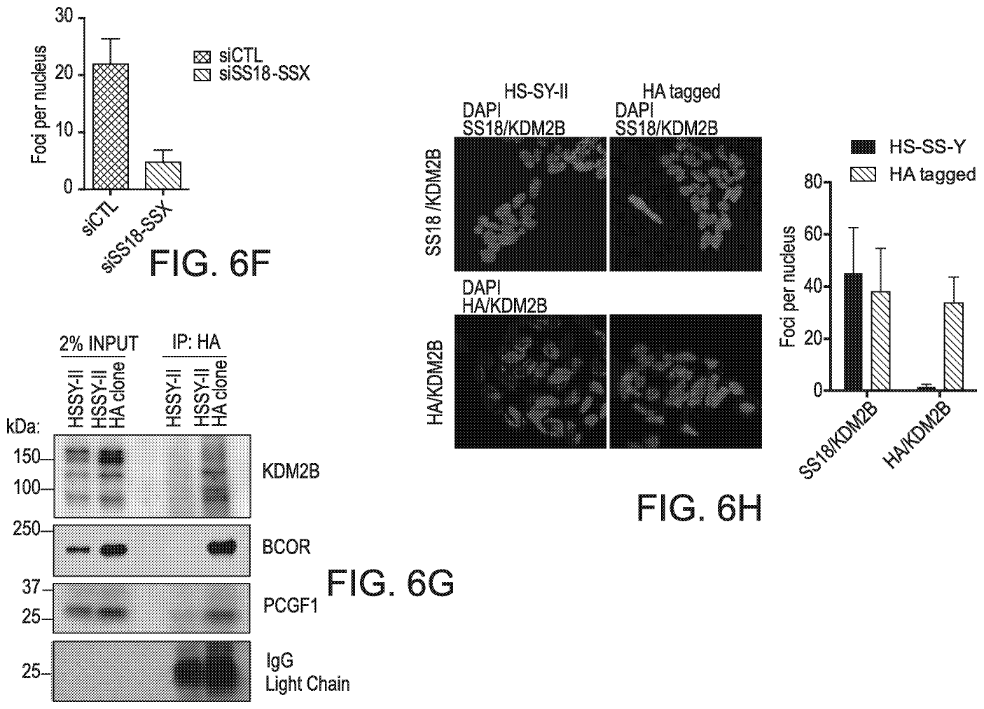

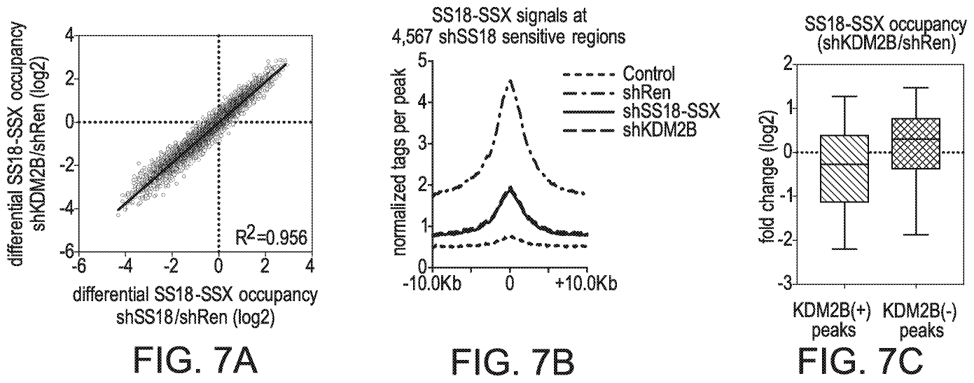

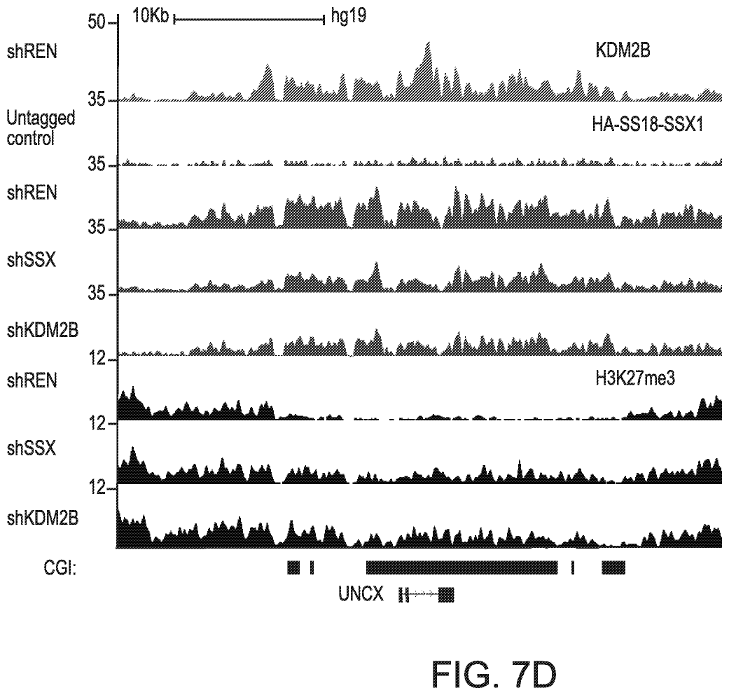

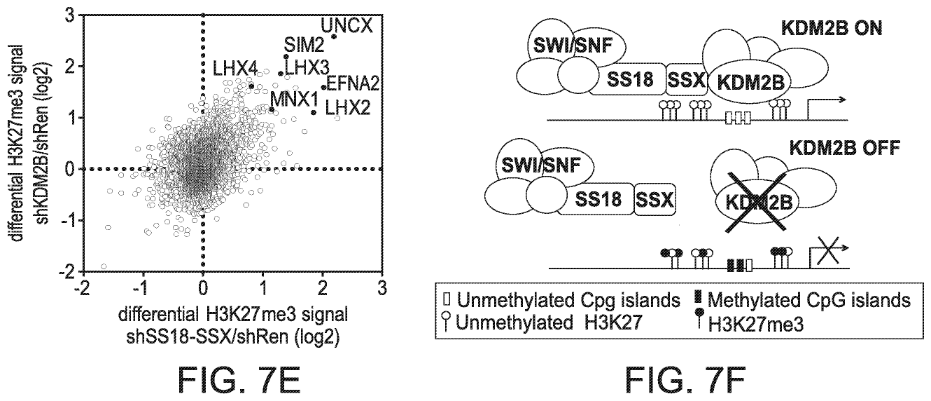

To evaluate the role of KDM2B protein domains we exploited the fact that in-frame deletions produced by CRISPR-Cas9-directed mutagenesis (which occur in ˜1/3 cases) are more deleterious to protein function when they disrupt critical domains (Shi et al., 2015); consequently, in competition assays, single guide RNAs (sgRNAs) targeting domains crucial for cell proliferation or survival are more depleted than those targeting dispensable regions. sgRNAs targeting the 5′ exons of KDM2B, the JmjC domain, as well as the ZF-CxxC domain (FIG. 4A) were evaluated in 5 human synovial sarcoma cell lines. sgRNAs were cloned by annealing two DNA oligos and ligating into a BsmB1-digested U6-sgRNA-EFS-GFP vector (Addgene #57822). sgRNAs in were designed using http://crispr.mit.edu/ and Benchling (https://benchling.com). The majority of sgRNAs used in this study had a quality score above 70 to minimize off-target effects. sgRNAs were designed to target 5′ coding exons of each target gene or functional domains of each protein based on the NCBI database annotation. Synovial sarcoma cell lines were transduced with lentiCas9-Blast (Addgene #52962) and selected using 5 ug/ml of blasticidin to generate stable Cas9-expressing cell lines. Cells were consequently transduced with pLKO5.sgRNA.EFS.GFP to about 80% transduction efficiency. Quantification of GFP fluorescent cells was monitored on a Guava Easycyte (Millipore) from day 3 following transduction. Fold depletion was calculated as previously described (Shi et al., 2015). T7 assays were performed to evaluate CRISPR/Cas9-mediated gene editing. Experiments were performed in two independent duplicates for all cell lines.

Tagging of Endogenous SS18-SSX1 Using CRISPR/Cas9-Mediated Homologous Directed Repair (HDR)

HS-SY-II cells were transfected using lipofectamine with pX458 (encoding Cas9, GFP and a sgRNA targeting the N-terminal region of the SS18 gene and a single stranded DNA template (ssDNA) containing ATG-FLAG-HA sequence flanked by homology arms to the N-terminal region of SS18. Three days following transfection cells were single-cell sorted into 96-well plates, for further analysis of cell clones. Clones were analyzed by immunofluorescence against HA-tag and PCR detection of targeted genomic regions using primers surrounding the ATG region of the SS18 gene. Positive clones were further evaluated by sequencing of PCR amplified genomic regions surrounding the SS18 N-terminal region.

Co-Immunoprecipitation

For co-immunoprecipitation a Nuclear complex Co-IP kit (Active Motif) was used. Cells were collected in cold PBS with phosphatase inhibitors and lysed in hypotonic buffer for 10 min. Isolated nuclei were further incubated in digestion buffer containing DNase for 90 min at 4 C. Nuclear lysates were cleared by centrifugation and quantified using DC Protein assay (BioRad); 250-500 μg of protein was incubated with 3 μg of antibody (KDM2B Millipore 09-864: HA-tag Cell Signaling 3956, normal rabbit IgG: Santa-Cruz Biotechnologies, sc-2027) in low stringency IP buffer containing 150 mM NaCl, 1% detergent and protease inhibitors; and incubated overnight at 4° C. with rotation. Next day Protein A/G magnetic beads were washed in low stringency IP buffer and incubated with the immunoprecipitation for 2 hours at 4° C. under rotation. Following incubation, beads were washed 3 times in low stringency IP buffer containing BSA and 3 times in low stringency IP buffer without BSA, and boiled in loading dye for 5 minutes, before western blot analysis. Antibodies against PCGF1 (Santa Cruz, 515371), SS18 (Santa Cruz, 390266), BCOR (Santa Cruz, sc-514576) KDM2B (Millipore, 09-864) and HA-tag (Cell Signaling, 3724) were used. For experiments using transient expression in 293T cells, gene fragments encoding HA tagged versions of SS18, SSX1 wild type versions, SS18-SSX1 C-terminal deletions mutants and GFP fusion, were cloned into EcoR1 and XhoI sites in MSCV-puro plasmid. 10 ug of plasmid was transfected into 293T cells using PEI (as described above), and cells were collected for co-IP analysis 48 hours after transfection.

Proximity Ligation Assay (PLA)

Indicated synovial sarcoma cell lines were seeded at 3×10⁴ cells/well in culture treated 8-well chamber slides and treated as previously described (Laporte et al., 2016). Primary antibodies for PLA were used at 1/1000 dilution: SS18 (Santa-Cruz Biotechnologies, sc-28698), KDM2B (Abnova, H00084678-M09), HA (Santa-Cruz Biotechnologies, sc-805). Proximity ligation was performed utilizing the Duolink® In Situ Red Starter Kit Mouse/Rabbit (Sigma-Aldrich, DUP92101-1KT) according to the manufacturer's protocol. Fluorescence was detected using a Zeiss Axioplan2 microscope at 40×. Images were quantified in triplicate using ImageJ software (NIH) as foci per nucleus, defined as the number of interaction points counted per nucleus. For PLA analysis upon SS18-SSX knockdown, duplex oligo (sense, CAAGAAGCCAGCAGAGGAATT; antisense, UUCCUCUGCUGGCUUCUUGTT) SS18-SSX siRNAs were designed to target the SSX portion of SS18-SSX using the Integrated. DNA Technologies RNA interference (RNAi) design tool, and synthesized by Integrated DNA Technologies (IDT) as previously described (Lubieniecka et al., 2008). HS-SY-II cells were seeded in 6-well plates. At 60% confluence, cells were transfected with 50 pmol siSS18-SSX and 9 μL Lipofectamine RNAiMAX transfection reagent (Invitrogen) in Opti-MEM serum free media (Life Technologies). Protein was harvested 48-hours post transfection, and knockdown confirmed by western blot with an SS18 antibody (Santa Cruz Biotechnologies, sc-28698).

Immunofluorescence and Immunoblotting.

Cells were seeded in 96-well imaging plates and fixed the following day with 4% paraformaldehyde (PFA) for 15 minutes. Permeabilization was performed using TritonX (0.1% in PBS) for 10 min followed by incubation with blocking solution (1% BSA, 0.1% Gelatin Fish in PBS) for 30 min. Incubation with Anti-HA-tag antibody (Cell Signaling, 3724, 1:1000) or α-SMA (Sigma clone 1A4, 1:2000) was performed in blocking buffer for 2 hours at RT. Cells were washed, incubated with secondary anti-rabbit Alexa-594 (Invitrogen) and counter stained with DAPI. Image acquisition was performed using IN Cell analyzer 6000 (GE Healthcare Life Sciences). For protein lysates cells were incubated with RIPA buffer supplemented with protease inhibitors (Protease inhibitor tablets, Roche) for 30 min and cleared by centrifugation (15 min 14.000 rpms 4C). Protein was quantified using the DC protein assay (BioRad). The following antibodies were used for immunoblotting: β-ACTIN (ac-15, Sigma), KDM2B (Millipore, 09-864) HA-tag (Cell Signaling, 3724) and Myc-tag (Cell Signaling, 2276).

RNA Expression Analysis

For quantitative RT-PCR, total RNA was isolated using the RNeasy Mini Kit (Qiagen), and cDNA was obtained using the TaqMan reverse transcription reagents (Applied Biosystems). Real-time PCR was performed in triplicate in two independent experiments using SYBR Green PCR Master Mix (Applied Biosystems) on the ViiA 7 Real-Time PCR System (Invitrogen). (3-actin served as an endogenous normalization control. Gene-specific primer sets for mouse and human sequences were designed using NCBI's qPrimerDepot (http://primerdepot.nci.nih.gov) or described elsewhere (Banito et al, 2009; Acosta et al, 2013; Kawai et al, 1998; Tzatsos et al, 2013; Hatzi et al, 2013). For high throughput RNA sequencing, total RNA from two independent experiments (and two shRNAs per gene) was extracted using an RNeasy minikit (Qiagen). Cells transduced with the indicated shRNAs were collected 12 days post-infection. RNA-Seq library construction and sequencing were performed at the integrated genomics operation (IGO) Core at MSKCC according to standard protocols. Poly-A selection was performed. For sequencing approximately 10 million 50 bp paired-end reads were acquired per replicate condition. Resulting RNA-Seq data was analyzed by removing adaptor sequences using Trimmomatic (Bolger et al., 2014). RNA-Seq reads were then aligned to GRCh37.75 (hg19) with STAR (Dobin et al., 2013) and genome-wide transcript counting was performed by HTSeq (Anders et al., 2015) to generate a matrix of fragments per kilobase of exon per million fragments mapped (RPKM). Gene expressions of RNA-Seq data were clustered using hierarchical clustering based on one minus Pearson correlation test using Morpheus (https://software.broadinstitute.org/morpheus/). Gene ontology of shSS18-SSX and shKDM2B co-regulated genes was performed using David functional annotation tool (https://david.ncifcrf.gov/); using a cut off of 2-fold difference in both conditions. For pathway enrichment analysis, the weighted GSEA Pre-ranked mode was used (http://www.broadinstitute.org/gsea).

Chromatin Immunoprecipitation (ChIP)

Chromatin immunoprecipitation was performed as previously described (Hatzi et al., 2013). Briefly, HS-SY-II cells were fixed with 1% formaldehyde for 15 min and the cross-linking reaction was stopped by adding 125 mM glycine. Cells were washed twice with cold PBS and lysed in swelling buffer (150 mM NaCl, 1% v/v Nonidet P-40, 0.5% w/v deoxycholate, 0.1% w/v SDS, 50 mM Tris pH8, 5 mM EDTA) supplemented with protease inhibitors. Cell lysates were sonicated using Covaris E220 Sonicator to generate fragments less than 400 bp. Sonicated lysates were centrifuged, and incubated overnight at 4° C. with specific antibodies (BRG1 Abcam 110641; KDM2B Millipore 17-10264, HA-tag Abcam 9110; H3K27me3 Millipore 07-449). Immunocomplexes were recovered by incubation with 30 ul protein A/G magnetic beads (Thermofisher) for 2 h at 4° C. Beads were sequentially washed twice with RIPA buffer, increasing stringency ChIP wash buffers (150 mM NaCl, 250 mM NaCl, 250 mM LiCl) and finally TE buffer. Immunocomplexes were eluted using elution buffer (1% SDS, 100 mM NaHCO₃) and cross-linking was reverted by addition of 300 mM NaCl and incubation at 65° C. overnight. DNA was purified using PCR purification kit (Qiagen). For HA-tag and BRG1 ChIP the same protocol was used with small modifications: cells were pre-fixed with ethylene glycol bis(succinimidyl succinate) (EGS) (Thermo Scientific) as previously described (Zeng et al., 2006) and the washing step containing 250 mM LiCl was omitted to increase yield without compromising specificity (as shown by absence of HA ChIP signal in HS-SY-II parental untagged cells). For ChIP-qPCRs a fraction of the ChIP product was used as template in 15 ul real time PCR reactions using SYBR Green PCR Master Mix (Applied Biosystems) on the ViiA 7 Real-Time PCR System (Invitrogen). Input chromatin was used for estimation of relative enrichment.

Assay for Transposase-Accessible Chromatin Sequencing (ATAC-Seq).

ATAC-Seq was performed as previously described (Buenrostro et al., 2013). Fifty thousand GFP positive cells were sorted by fluorescence-activated cell sorting (FACS). Cells were lysed in lysis buffer (10 mM Tris, pH 7.4; 10 mM NaCl; 3 mM MgCl₂; 0.1% (v/v) IGEPAL) and centrifuged for 10 min (500×g) to isolate the nuclear fraction. Transposition reaction was performed for 30 minutes at 37° C. using the Tn5 Transposase kit from Nextera accordingly to the manufacturer's instructions. Transposed DNA fragments were amplified by PCR using barcoded primers (Buenrostro et al., 2013) and the NEBNext High Fidelity 2X master mix (12 PCR cycles). Amplified libraries were purified using Qiagen MinElute, analyzed using Bioanalyser and combined for Illumina High-throughput sequencing.

ChIP-Seq Library Preparation, Illumina Data Analysis and Peak Detection.

ChIP-Seq libraries were prepared at the Center for Epigenetic Research (MSKCC) using the NEBNext® ChIP-Seq Library Prep Master Mix Set for Illumina® (New England BioLabs) following the manufacturer's instructions. Raw reads were mapped to the reference human genome assembly GRCh37 (hg19) using Bowtie and SAMtools. For further analyses, HOMER suit of tools was used (Heinz et al., 2010). Aligned bam files were subjected to peak calling using findPeaks tools with the default setting, except -style histone was implemented to find for broad regions of H3K27me3 peaks. Once ChIP-Seq peaks were identified from HA-SS18/SSX and H3K27me3 ChIP-Seq experiments with individual shRNA expression, all peaks were combined and merged if the maximum distance between peak centers were smaller than 100 bp. With these combined peaks, tag counts were re-calculated and normalized to 10 million reads. The peaks with lower than 50 tag counts of HA-SS18/SSX or H3K27me3 were excluded from further analyses, which finally yielded union peaks of HA-SS18-SSX (n=10,984) and H3K27me3 (n=13,226) ChIP-Seq experiments.

To identify HA-SS18/SSX and KDM2B co-occupancy, tag counts of KDM2B ChIP-Seq experiments were calculated from 10,984 peaks of HA-SS18/SSX ChIP-Seq. If the normalized tag counts of KDM2B were lower than 20, they were considered as HA-SS18/SSX (+) KDM2B (−) regions (n=451), and, if not, considered as HA-SSX/SSX (+) KDM2B (+) regions (n=10,533). To visualize ChIP-Seq tracks, normalized bigWig files were generated with makeBigWig tool. To create metagene plots in FIG. 13A, +/−10 kb from peak center was aligned and binned with 25 bp with annotate Peaks tool, then visualized with Java Tree view. To generate custom gene sets from ChIP-Seq data, genes with the closest TSS from each ChIP-Seq peak were assigned as peak-associated genes or using the GREAT tool as explained below.

Gene Ontology (GO) Analysis of ChIP-Seq Regions.

GREAT tools were used to predict GO terms of ChIP-Seq enriched regions (http://bejerano.stanford.edu/great/public/html/index.php). 10,533 SS18-SSX and KDM2B co-occupied regions were tested to define associated genes suing GREAT's default settings (basal plus extension; proximal 5,000-bp upstream and 1,000-bp downstream) but using a more stringent distal extension of 100 kb instead of 1000 kb to include distal regulatory regions but minimize chances of assigning incorrect genes to each regulatory region. This analysis revealed a total of 3883 genes associated with 5518-SSX/KDM2B-bound regions. GO terms from the GO consortium (http://geneontology.org) the molecular signature database (http://software.broadinstitute.org/gsea/msigdb) were plotted as bar graphs with binomial −log10 p-values (FIG. 13G). A total of 3928 genes were assigned to 10,984 SS18-SSX-bound regions.

Immunohistochemistry of Sarcoma Tissue Microarrays (TMAs).

Immunohistochemical staining was performed using the Ventana DISCOVERY® ULTRA semi-automated staining system (Ventana Medical Systems Inc, Tucson, Ariz.). Briefly, heat-induced antigen retrieval was performed using the standard Cell Conditioning 1 (CC1, Ventana) protocol. Sections were incubated with goat anti-KDM2B polyclonal antibody (S-15, SantaCruz Biotechnology Inc, Santa Cruz, Calif.) at 1:25 dilution in DISCOVERY antibody diluent (Ventana) for 2 h at room temperature, followed by incubation with AffiniPure rabbit anti-goat IgG (H+L) unconjugated secondary antibody (Jackson ImmunoResearch Laboratories Inc, West Grove, Pa.) for 32 min at 37° C. Chromogen visualization was performed using the ChromoMap DAB Kit UltraMap anti-rabbit tertiary antibody (Ventana). Slides were counterstained with hematoxylin and mounted. Digital images of immunostained tissue microarrays were acquired using a BLISS imaging microscope (Bacus Laboratories, Lombard, Ill., USA). A pathologist experienced in scoring biomarkers in bone and soft tissue tumors scored each core separately, and the average score from all cores of the same tumor was calculated. KDM2B immunopositivity was scored in a semi-quantitative manner for the intensity (0=negative, 1=weak positive, 2=moderate positive, 3=strong positive) and for the percentage of positive sarcoma cell nuclei. These were multiplied to generate an H-score. A Kruskal-Wallis 1-way ANOVA test was used to assess the differences between histological sub-types. Statistically significant difference was defined as p<0.05. Data analysis was performed using IBM® SPSS® statistics software (version 26).

Analysis of DNA Methylation and RNA-Seq Data from TCGA.

RNA-Seq and DNA methylation data from the Illumina Human Methylation 450K platform was downloaded from the UCSC Cancer Genome Browser (Zhu et al., 2009) for 206 samples in the TCGA Sarcoma cohort (“Comprehensive and Integrated Characterization of Adult Soft Tissue Sarcomas”, submitted), as described in Experimental model and subject details. We discarded all the probes that were masked as NA (‘Not Available’) for more than 90% of the TCGA samples. A probe is masked as NA at level three of the TCGA database if (a) the detection p-value is greater than 0.05 (which means that the measured signal is not significantly different from background), (b) the probe contains known SNPs after comparison with the dbSNP database or (c) the probe contains DNA sequences of known repetitive elements in more than 10 bp of each 50 bp probe sequence. A total of 6,412 regions of the 10,533 regions co-occupied by both SS18-SSX and KDM2B overlapped with at least one of the Illumina probes that remained in the array. For the results shown in FIG. 13E, we calculated average DNA methylation levels per sample in co-occupied regions by first computing the average beta value over the 61,577 probes that overlap any of the 6,412 co-occupied regions. DNA methylation levels per sample outside of co-occupied regions were estimated as the average beta value over all the 309,688 probes that did not overlap any of the 6,412 co-occupied regions. For the results shown in FIG. 19D, we computed average levels of DNA methylation per region across the 10 synovial samples in the TCGA cohort for each one of the 6,412 regions that overlapped at least one probe in the array. We then compared average levels on DNA methylation in the 500 co-occupied regions with the highest minimal co-occupancy score vs. average DNA methylation levels in the 500 co-occupied regions with the lowest minimal co-occupancy score. For each region, the minimal co-occupancy score was defined as the minimum value in the pair of SS18-SSX and KDM2B ChIP occupancy scores.

Quantification and Statistical Analysis

Data are expressed as mean±s.d or ±s.e.m as indicated in the figure legends. Group size was determined on the basis of the results of preliminary experiments and no statistical method was used to predetermine sample size. The indicated sample size (n) represents biological replicates. Group allocation and outcome assessment were not performed in a blinded manner. All samples that met proper experimental conditions were included in the analysis. Statistical significance was determined by two-tailed Student's t-test, and Pearson's correlation using Prism 6 software (GraphPad Software). Significance was set at P<0.05.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Aguirre, A. J., Meyers, R. M., Weir, B. A., Vazquez, F., Zhang, C. Z., Ben-David, U., Cook, A., Ha, G., Harrington, W. F., Doshi, M. B., et al. (2016). Genomic Copy Number Dictates a Gene-Independent Cell Response to CRISPR/Cas9 Targeting. Cancer Discov 6, 914-929.
Anders, S., Pyl, P. T., and Huber, W. (2015). HTSeq—a Python framework to work with high-throughput sequencing data. Bioinformatics 31, 166-169.
Andricovich, J., Kai, Y., Peng, W., Foudi, A., and Tzatsos, A. (2016). Histone demethylase KDM2B regulates lineage commitment in normal and malignant hematopoiesis. J Clin Invest 126, 905-920.
Baird, K., Davis, S., Antonescu, C. R., Harper, U. L., Walker, R. L., Chen, Y., Glatfelter, A. A., Duray, P. H., and Meltzer, P. S. (2005). Gene expression profiling of human sarcomas: insights into sarcoma biology. Cancer Res 65, 9226-9235.
Barham, W., Frump, A. L., Sherrill, T. P., Garcia, C. B., Saito-Diaz, K., VanSaun, M. N., Fingleton, B., Gleaves, L., Orton, D., Capecchi, M. R., et al. (2013). Targeting the Wnt pathway in synovial sarcoma models. Cancer Discov 3, 1286-1301.
Benton, M. C., Johnstone, A., Eccles, D., Harmon, B., Hayes, M. T., Lea, R. A., Griffiths, L., Hoffman, E. P., Stubbs, R. S., and Macartney-Coxson, D. (2015). An analysis of DNA methylation in human adipose tissue reveals differential modification of obesity genes before and after gastric bypass and weight loss. Genome Biol 16, 8.
Blackledge, N. P., Farcas, A. M., Kondo, T., King, H. W., McGouran, J. F., Hanssen, L. L., Ito, S., Cooper, S., Kondo, K., Koseki, Y., et al. (2014). Variant PRC1 complex-dependent H2A ubiquitylation drives PRC2 recruitment and polycomb domain formation. Cell 157, 1445-1459.
Bolger, A. M., Lohse, M., and Usadel, B. (2014). Trimmomatic: a flexible trimmer for Illumina sequence data. Bioinformatics 30, 2114-2120.
Boulard, M., Edwards, J. R., and Bestor, T. H. (2015). FBXL10 protects Polycomb-bound genes from hypermethylation. Nat Genet 47, 479-485.
Buenrostro, J. D., Giresi, P. G., Zaba, L. C., Chang, H. Y., and Greenleaf, W. J. (2013). Transposition of native chromatin for fast and sensitive epigenomic profiling of open chromatin, DNA-binding proteins and nucleosome position. Nat Methods 10, 1213-1218.
Cheng, H., Dodge, J., Mehl, E., Liu, S., Poulin, N., van de Rijn, M., and Nielsen, T. O. (2009). Validation of immature adipogenic status and identification of prognostic biomarkers in myxoid liposarcoma using tissue microarrays. Hum Pathol 40, 1244-1251.
Clark, J., Rocques, P. J., Crew, A. J., Gill, S., Shipley, J., Chan, A. M., Gusterson, B. A., and Cooper, C. S. (1994). Identification of novel genes, SYT and SSX, involved in the t(X;18)(p11.2;q11.2) translocation found in human synovial sarcoma. Nat Genet 7, 502-508.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21
dos Santos, N. R., de Bruijn, D. R., Kater-Baats, E., Otte, A. P., and van Kessel, A. G. (2000). Delineation of the protein domains responsible for SYT, SSX, and SYT-SSX nuclear localization. Exp Cell Res 256, 192-202.
Endo, M., de Graaff, M. A., Ingram, D. R., Lim, S., Lev, D. C., Briaire-de Bruijn, I. H., Somaiah, N., Bovee, J. V., Lazar, A. J., and Nielsen, T. O. (2015). NY-ESO-1 (CTAG1B) expression in mesenchymal tumors. Mod Pathol 28, 587-595.
Endo, M., Su, L., and Nielsen, T. O. (2014). Activating transcription factor 2 in mesenchymal tumors. Hum Pathol 45, 276-284.
Farcas, A. M., Blackledge, N. P., Sudbery, I., Long, H. K., McGouran, J. F., Rose, N. R., Lee, S., Sims, D., Cerase, A., Sheahan, T. W., et al. (2012). KDM2B links the Polycomb Repressive Complex 1 (PRC1) to recognition of CpG islands. Elife 1, e00205.
Fellmann, C., Hoffmann, T., Sridhar, V., Hopfgartner, B., Muhar, M., Roth, M., Lai, D. Y., Barbosa, I. A., Kwon, J. S., Guan, Y., et al. (2013). An optimized microRNA backbone for effective single-copy RNAi. Cell Rep 5, 1704-1713.
Gao, Z., Zhang, J., Bonasio, R., Strino, F., Sawai, A., Parisi, F., Kluger, Y., and Reinberg, D. (2012). PCGF homologs, CBX proteins, and RYBP define functionally distinct PRC1 family complexes. Mol Cell 45, 344-356.
Haldar, M., Hancock, J. D., Coffin, C. M., Lessnick, S. L., and Capecchi, M. R. (2007). A conditional mouse model of synovial sarcoma: insights into a myogenic origin. Cancer Cell 11, 375-388.
Hatzi, K., Jiang, Y., Huang, C., Garrett-Bakelman, F., Gearhart, M. D., Giannopoulou, E. G., Zumbo, P., Kirouac, K., Bhaskara, S., Polo, J. M., et al. (2013). A hybrid mechanism of action for BCL6 in B cells defined by formation of functionally distinct complexes at enhancers and promoters. Cell Rep 4, 578-588.
He, J., Kallin, E. M., Tsukada, Y., and Zhang, Y. (2008). The H3K36 demethylase Jhdm1b/Kdm2b regulates cell proliferation and senescence through p15(Ink4b). Nat Struct Mol Biol 15, 1169-1175.
He, J., Shen, L., Wan, M., Taranova, O. Wu, H., and Zhang, Y. (2013). Kdm2b maintains murine embryonic stem cell status by recruiting PRC1 complex to CpG islands of developmental genes. Nat Cell Biol 15, 373-384.
Hegarty, S. V., Sullivan, A. M., and O'Keeffe, G. W. (2013). Midbrain dopaminergic neurons: a review of the molecular circuitry that regulates their development. Dev Biol 379, 123-138.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Helman, L. J., and Meltzer, P. (2003). Mechanisms of sarcoma development. Nat Rev Cancer 3, 685-694.
Ishibe, T., Nakayama, T., Okamoto, T., Aoyama, T., Nishijo, K., Shibata, K. R., Shima, Y., Nagayama, S., Katagiri, T., Nakamura, Y., et al. (2005). Disruption of fibroblast growth factor signal pathway inhibits the growth of synovial sarcomas: potential application of signal inhibitors to molecular target therapy. Clin Cancer Res 11, 2702-2712.
Junco, S. E., Wang, R., Gaipa, J. C., Taylor, A. B., Schirf, V., Gearhart, M. D., Bardwell, V. J., Demeler, B., Hart, P. J., and Kim, C. A. (2013). Structure of the polycomb group protein PCGF1 in complex with BCOR reveals basis for binding selectivity of PCGF homologs. Structure 21, 665-671.
Kadoch, C., and Crabtree, G. R. (2013). Reversible disruption of mSWI/SNF (BAF) complexes by the SS18-SSX oncogenic fusion in synovial sarcoma. Cell 153, 71-85.
Kadoch, C., Williams, R. T., Calarco, J. P., Miller, E. L., Weber, C. M., Braun, S. M., Pulice, J. L., Chory, E. J., and Crabtree, G. R. (2017). Dynamics of BAF-Polycomb complex opposition on heterochromatin in normal and oncogenic states. Nat Genet 49, 213-222.
Kao, Y. C., Sung, Y. S., Zhang, L., Jungbluth, A. A., Huang, S. C., Argani, P., Agaram, N. P., Zin, A., Alaggio, R., and Antonescu, C. R. (2016). BCOR Overexpression Is a Highly Sensitive Marker in Round Cell Sarcomas With BCOR Genetic Abnormalities. Am J Surg Pathol 40, 1670-1678.
Kao, Y. C., Sung, Y. S., Zhang, L., Kenan, S., Singer, S., Tap, W. D., Swanson, D., Dickson, B. C., and Antonescu, C. R. (2017). BCOR upregulation in a poorly differentiated synovial sarcoma with SS18L1-SSX1 fusion-A pathologic and molecular pitfall. Genes Chromosomes Cancer 56, 296-302.
Kawai, A., Naito, N., Yoshida, A., Morimoto, Y., Ouchida, M., Shimizu, K., and Beppu, Y. (2004). Establishment and characterization of a biphasic synovial sarcoma cell line, SYO-1. Cancer Lett 204, 105-113.
Kim, K. H., Kim, W., Howard, T. P., Vazquez, F., Tsherniak, A., Wu, J. N., Wang, W., Haswell, J. R., Walensky, L. D., Hahn, W. C., et al. (2015). SWI/SNF-mutant cancers depend on catalytic and non-catalytic activity of EZH2. Nat Med 21, 1491-1496.
Ladanyi, M., Antonescu, C. R., Leung, D. H., Woodruff, J. M., Kawai, A., Healey, J. H., Brennan, M. F., Bridge, J. A., Neff, J. R., Barr, F. G., et al. (2002). Impact of SYT-SSX fusion type on the clinical behavior of synovial sarcoma: a multi-institutional retrospective study of 243 patients. Cancer Res 62, 135-140.
Laporte, A. N., Ji, J. X., Ma, L., Nielsen, T. O., and Brodin, B. A. (2016). Identification of cytotoxic agents disrupting synovial sarcoma oncoprotein interactions by proximity ligation assay. Oncotarget 7, 34384-34394.
Lee, S. K., and Pfaff, S. L. (2001). Transcriptional networks regulating neuronal identity in the developing spinal cord. Nat Neurosci 4 Suppl, 1183-1191.
Lim, F. L., Soulez, M., Koczan, D., Thiesen, H. J., and Knight, J. C. (1998). A KRAB-related domain and a novel transcription repression domain in proteins encoded by SSX genes that are disrupted in human sarcomas. Oncogene 17, 2013-2018.
Long, H. K., Blackledge, N. P., and Klose, R. J. (2013). ZF-CxxC domain-containing proteins, CpG islands and the chromatin connection. Biochem Soc Trans 41, 727-740.
Lubieniecka, J. M., de Bruijn, D. R., Su, L., van Dijk, A. H., Subramanian, S., van de Rijn, M., Poulin, N., van Kessel, A. G., and Nielsen, T. O. (2008). Histone deacetylase inhibitors reverse SS18-SSX-mediated polycomb silencing of the tumor suppressor early growth response 1 in synovial sarcoma. Cancer Res 68, 4303-4310.
Mertens, F., Antonescu, C. R., and Mitelman, F. (2016). Gene fusions in soft tissue tumors: Recurrent and overlapping pathogenetic themes. Genes Chromosomes Cancer 55, 291-310.
Nagai, M., Tanaka, S., Tsuda, M., Endo, S., Kato, H., Sonobe, H., Minami, A., Hiraga, H., Nishihara, H., Sawa, H., et al. (2001). Analysis of transforming activity of human synovial sarcoma-associated chimeric protein SYT-SSX1 bound to chromatin remodeling factor hBRM/hSNF2 alpha. Proc Natl Acad Sci USA 98, 3843-3848.
Nagayama, S., Katagiri, T., Tsunoda, T., Hosaka, T., Nakashima, Y., Araki, N., Kusuzaki, K., Nakayama, T., Tsuboyama, T., Nakamura, T., et al. (2002). Genome-wide analysis of gene expression in synovial sarcomas using a cDNA microarray. Cancer Res 62, 5859-5866.
Naka, N., Takenaka, S., Araki, N., Miwa, T., Hashimoto, N., Yoshioka, K., Joyama, S., Hamada, K., Tsukamoto, Y., Tomita, Y., et al. (2010). Synovial sarcoma is a stem cell malignancy. Stem Cells 28, 1119-1131.
Ng, T. L., Gown, A. M., Barry, T. S., Cheang, M. C., Chan, A. K., Turbin, D. A., Hsu, F. D., West, R. B., and Nielsen, T. O. (2005). Nuclear beta-catenin in mesenchymal tumors. Mod Pathol 18, 68-74.
Nielsen, T. O., Hsu, F. D., O'Connell, J. X., Gilks, C. B., Sorensen, P. H., Linn, S., West, R. B., Liu, C. L., Botstein, D., Brown, P. O., et al. (2003). Tissue microarray validation of epidermal growth factor receptor and SALL2 in synovial sarcoma with comparison to tumors of similar histology. Am J Pathol 163, 1449-1456.
Nielsen, T. O., Poulin, N. M., and Ladanyi, M. (2015). Synovial sarcoma: recent discoveries as a roadmap to new avenues for therapy. Cancer Discov 5, 124-134.
Nojima, T., Wang, Y. S., Abe, S., Matsuno, T., Yamawaki, S., and Nagashima, K. (1990). Morphological and cytogenetic studies of a human synovial sarcoma xenotransplanted into nude mice. Acta Pathol Jpn 40, 486-493.
Pacheco, M., and Nielsen, T. O. (2012). Histone deacetylase 1 and 2 in mesenchymal tumors. Mod Pathol 25, 222-230.
Peters, T. L. (2015). BCOR-CCNB3 fusions are frequent in undifferentiated sarcomas of male children. Mod Pathol 28, 575-586.
Pierron, G. (2012). A new subtype of bone sarcoma defined by BCOR-CCNB3 gene fusion. Nat Genet 44, 461-466.
Roberts, C. W., and Orkin, S. H. (2004). The SWI/SNF complex—chromatin and cancer. Nat Rev Cancer 4, 133-142.
Roy, A., Kumar, V., Zorman, B., Fang, E., Haines, K. M., Doddapaneni, H., Hampton, O. A., White, S., Bavle, A. A., Patel, N. R., et al. (2015). Recurrent internal tandem duplications of BCOR in clear cell sarcoma of the kidney. Nat Commun 6, 8891.
Sanchez, C., Sanchez, I., Demmers, J. A., Rodriguez, P., Strouboulis, J., and Vidal, M. (2007). Proteomics analysis of Ring1B/Rnf2 interactors identifies a novel complex with the Fbxl10/Jhdm1B histone demethylase and the Bcl6 interacting corepressor. Mol Cell Proteomics 6, 820-834.
Sheffield, N. C., Pierron, G., Klughammer, J., Datlinger, P., Schonegger, A., Schuster, M., Hadler, J., Surdez, D., Guillemot, D., Lapouble, E., et al. (2017). DNA methylation heterogeneity defines a disease spectrum in Ewing sarcoma. Nat Med 23, 386-395.
Shi, J., Wang, E., Milazzo, J. P., Wang, Z., Kinney, J. B., and Vakoc, C. R. (2015). Discovery of cancer drug targets by CRISPR-Cas9 screening of protein domains. Nat Biotechnol 33, 661-667.
Singer, S., Demetri, G. D., Baldini, E. H., and Fletcher, C. D. (2000). Management of soft-tissue sarcomas: an overview and update. Lancet Oncol 1, 75-85.
Soderberg, O., Gullberg, M., Jarvius, M., Ridderstrale, K., Leuchowius, K. J., Jarvius, J., Wester, K., Hydbring, P., Bahram, F., Larsson, L. G., et al. (2006). Direct observation of individual endogenous protein complexes in situ by proximity ligation. Nat Methods 3, 995-1000.
Sonobe, H., Manabe, Y., Furihata, M., Iwata, J., Oka, T., Ohtsuki, Y., Mizobuchi, H., Yamamoto, H., Kumano, O., and Abe, S. (1992). Establishment and characterization of a new human synovial sarcoma cell line, HS-SY-II. Lab Invest 67, 498-505.
Soulez, M., Saurin, A. J., Freemont, P. S., and Knight, J. C. (1999). SSX and the synovial-sarcoma-specific chimaeric protein SYT-SSX co-localize with the human Polycomb group complex. Oncogene 18, 2739-2746.
Specht, K., Zhang, L., Sung, Y. S., Nucci, M., Dry, S., Vaiyapuri, S., Richter, G. H., Fletcher, C. D., and Antonescu, C. R. (2016). Novel BCOR-MAML3 and ZC3H7B-BCOR Gene Fusions in Undifferentiated Small Blue Round Cell Sarcomas. Am J Surg Pathol 40, 433-442.
Stanton, B. Z., Hodges, C., Calarco, J. P., Braun, S. M., Ku, W. L., Kadoch, C., Zhao, K., and Crabtree, G. R. (2017). Smarca4 ATPase mutations disrupt direct eviction of PRC1 from chromatin. Nat Genet 49, 282-288.
Sturm, D., Orr, B. A., Toprak, U. H., Hovestadt, V., Jones, D. T., Capper, D., Sill, M., Buchhalter, I., Northcott, P. A., Leis, I., et al. (2016). New Brain Tumor Entities Emerge from Molecular Classification of CNS-PNETs. Cell 164, 1060-1072.
Su, L., Sampaio, A. V., Jones, K. B., Pacheco, M., Goytain, A., Lin, S., Poulin, N., Yi, L., Rossi, F. M., Kast, J., et al. (2012). Deconstruction of the SS18-SSX fusion oncoprotein complex: insights into disease etiology and therapeutics. Cancer Cell 21, 333-347.
Tamaki, S., Fukuta, M., Sekiguchi, K., Jin, Y., Nagata, S., Hayakawa, K., Hineno, S., Okamoto, T., Watanabe, M., Woltjen, K., et al. (2015). SS18-SSX, the Oncogenic Fusion Protein in Synovial Sarcoma, Is a Cellular Context-Dependent Epigenetic Modifier. PLoS One 10, e0142991.
Terry, J., Saito, T., Subramanian, S., Ruttan, C., Antonescu, C. R., Goldblum, J. R., Downs-Kelly, E., Corless, C. L., Rubin, B. P., van de Rijn, M., et al. (2007). TLE1 as a diagnostic immunohistochemical marker for synovial sarcoma emerging from gene expression profiling studies. Am J Surg Pathol 31, 240-246.
Thaete, C., Brett, D., Monaghan, P., Whitehouse, S., Rennie, G., Rayner, E., Cooper, C. S., and Goodwin, G. (1999). Functional domains of the SYT and SYT-SSX synovial sarcoma translocation proteins and co-localization with the SNF protein BRM in the nucleus. Hum Mol Genet 8, 585-591.
Trautmann, M., Sievers, E., Aretz, S., Kindler, D., Michels, S., Friedrichs, N., Renner, M., Kirfel, J., Steiner, S., Huss, S., et al. (2014). SS18-SSX fusion protein-induced Wnt/beta-catenin signaling is a therapeutic target in synovial sarcoma. Oncogene 33, 5006-5016.
Tzatsos, A., Pfau, R., Kampranis, S. C., and Tsichlis, P. N. (2009). Ndy1/KDM2B immortalizes mouse embryonic fibroblasts by repressing the Ink4a/Arf locus. Proc Natl Acad Sci USA 106, 2641-2646.
Ueno-Yokohata, H., Okita, H., Nakasato, K., Akimoto, S., Hata, J., Koshinaga, T., Fukuzawa, M., and Kiyokawa, N. (2015). Consistent in-frame internal tandem duplications of BCOR characterize clear cell sarcoma of the kidney. Nat Genet 47, 861-863.
Wu, X., Johansen, J. V., and Helin, K. (2013). Fbxl10/Kdm2b recruits polycomb repressive complex 1 to CpG islands and regulates H2A ubiquitylation. Mol Cell 49, 1134-1146.
Zeng, P. Y., Vakoc, C. R., Chen, Z. C., Blobel, G. A., and Berger, S. L. (2006). In vivo dual cross-linking for identification of indirect DNA-associated proteins by chromatin immunoprecipitation. Biotechniques 41, 694, 696, 698.
Zhu, J., Sanborn, J. Z., Benz, S., Szeto, C., Hsu, F., Kuhn, R. M., Karolchik, D., Archie, J., Lenburg, M. E., Esserman, L. J., et al. (2009). The UCSC Cancer Genomics Browser. Nat Methods 6, 239-240.
Zuber, J., McJunkin, K., Fellmann, C., Dow, L. E., Taylor, M. J., Hannon, G. J., and Lowe, S. W. (2011a). Toolkit for evaluating genes required for proliferation and survival using tetracycline-regulated RNAi. Nat Biotechnol 29, 79-83.
Zuber, J., Shi, J., Wang, E., Rappaport, A. R., Herrmann, H., Sison, E. A., Magoon, D., Qi, J., Blatt, K., Wunderlich, M., et al. (2011b). RNAi screen identifies Brd4 as a therapeutic target in acute myeloid leukaemia. Nature 478, 524-528.

1. A method of treating sarcoma in a subject in need thereof comprising administering an effective amount of a PRC1.1 inhibitory agent to the subject,
optionally wherein the PRC1.1 inhibitory agent is a polypeptide, a small molecule, a nucleic acid, a shRNA, an antibody agent, a KDM2B inhibitory agent, a BCOR inhibitory agent, or a PCGF1 inhibitory agent, and optionally wherein the sarcoma is characterized by a SS18-SSX fusion protein.

2.-4. (canceled)

5. The method of claim 1, wherein the PRC1.1 inhibitory agent
reduces interaction of the SS18-SSX fusion protein with a polycomb repressive complex;
reduces transcriptional activity induced by the SS18-SSX fusion protein; or
reduces interaction of the SS18-SSX fusion protein with CpG islands.

6.-8. (canceled)

9. The method of claim 1, wherein the sarcoma is synovial sarcoma.

10.-12. (canceled)

13. The method of claim 1, wherein administration of the PRC1.1 inhibitory agent results in
reduced proliferation of cancer cells;
cell cycle arrest; or
differentiation of synovial sarcoma cells into a more mesenchymal like state.

14.-15. (canceled)

16. The method of claim 13, wherein increased expression of COL1A1, SERPINE1 (PAI-1), ACTA2 (α-SMA), CDKN1A and/or CDKN2B indicate a more mesenchymal like state.

17. A method of treating sarcoma comprising administering an effective amount of a KDM2B inhibitory agent to a subject suffering from or susceptible to sarcoma, optionally wherein the KDM2B inhibitory agent is a polypeptide, a small molecule, a nucleic acid, a shRNA, or an antibody agent.

18. The method of claim 17, wherein the a KDM2B inhibitory agent is administered to a subject in whom a KDM2B dependency or a KDM2B-SS18-SSX dependency has been detected.

19. (canceled)

20. The method of claim 17, wherein the KDM2B inhibitory agent
reduces the level or activity of KDM2B;
reduces the interaction of KDM2B and SS18-SSX; or
reduces the interaction of KDM2B and PRC1.

21. The method of claim 17, wherein the KDM2B inhibitory agent targets the ZF-CXXC domain of KDM2B.

22.-23. (canceled)

24. The method of claim 17, wherein the sarcoma is characterized by KDM2B dependency or by decreased methylation at Histone 3 lysine 27 trimethylation (H3K27me3) relative to a reference.

25. (canceled)

26. The method of claim 24, wherein the reference is healthy tissue from the subject.

27. (canceled)

28. The method of claim 17, wherein the sarcoma is synovial sarcoma.

29.-31. (canceled)

32. The method of claim 17, wherein administration of the KDM2B inhibitory agent results in reduced proliferation of cancer cells.

33. A method of detecting KDM2B dependency in a subject comprising detecting H3K27me3 levels or the interaction between KDM2B and SS18-SSX in a sarcoma sample obtained from the subject.

34. (canceled)

35. The method of claim 33, wherein the H3K27me3 levels are decreased in the subject relative to a reference.

36. The method of claim 35, wherein the reference is healthy tissue from the subject.

37. (canceled)

38. The method of claim 1, wherein the PRC1.1 inhibitory agent targets a RAWUL domain.

39. A method of identifying a PRC1.1 inhibitory agent comprising detecting whether an agent disrupts the association of a SS18-SSX fusion protein with a component of a PRC1.1 complex.

40. (canceled)
